SEQUENCE LISTING (12 sequences)

SEQ ID NO: 1 - DNA, 41 bp, Artificial Sequence
  Synthetic polynucleotide
gtcatttgaagatatccaccgttatcgcgagcagattaaga

SEQ ID NO: 2 - DNA, 41 bp, Artificial Sequence
  Synthetic polynucleotide
tcttaatctgctcgcgataacggtggatatcttcaaatgac

SEQ ID NO: 3 - DNA, 41 bp, Artificial Sequence
  Synthetic polynucleotide
tcatttgaagatatccaccagtatcgcgagcagattaagag

SEQ ID NO: 4 - DNA, 41 bp, Artificial Sequence
  Synthetic polynucleotide
ctcttaatctgctcgcgatactggtggatatcttcaaatga

SEQ ID NO: 5 - DNA, 41 bp, Artificial Sequence
  Synthetic polynucleotide
agtcatttgaagatatccacgattatcgcgagcagattaag

SEQ ID NO: 6 - DNA, 41 bp, Artificial Sequence
  Synthetic polynucleotide
cttaatctgctcgcgataatcgtggatatcttcaaatgact

SEQ ID NO: 7 - DNA, 39 bp, Artificial Sequence
  Synthetic polynucleotide
gaagagtactccgcaatgagcgatcaatacatgaggacg

SEQ ID NO: 8 - DNA, 39 bp, Artificial Sequence
  Synthetic polynucleotide
cgtcctcatgtattgatcgctcattgcggagtactcttc

SEQ ID NO: 9 - DNA, 42 bp, Artificial Sequence
  Synthetic polynucleotide
cgaagtcatttgaagatatccaccattgtcgcgagcagatta

SEQ ID NO: 10 - DNA, 42 bp, Artificial Sequence
  Synthetic polynucleotide
taatctgctcgcgacaatggtggatatcttcaaatgacttcg

SEQ ID NO: 11 - DNA, 41 bp, Artificial Sequence
  Synthetic polynucleotide
cgaagtcatttgaagatatccaccatgatcgcgagcagatt

SEQ ID NO: 12 - DNA, 41 bp, Artificial Sequence
  Synthetic polynucleotide
aatctgctcgcgatcatggtggatatcttcaaatgacttcg

(19) United States
(12) Patent Application Publication
(10) Pub. No.: US 2024/0366567 A1
(43) Pub. Date: Nov. 7, 2024

(54) PHARMACEUTICAL COMBINATIONS COMPRISING A KRAS G12C INHIBITOR AND USES THEREOF FOR THE TREATMENT OF CANCERS

(71) Applicant: NOVARTIS AG, Basel (CH)

(72) Inventors: Saskia Maria BRACHMANN, Basel (CH); Simona COTESTA, Basel (CH); Xiaoming CUI, East Hanover, NJ (US); Ruben DE KANTER, Basel (CH); Anna FARAGO, Cambridge, MA (US); Marc GERSPACHER, Basel (CH); Diana GRAUS PORTA, Basel (CH); Jaeyeon KIM, Cambridge, MA (US); Catherine LEBLANC, Basel (CH); Edwige Liliane Jeanne LORTHIOIS, Basel (CH); Rainer MACHAUER, Basel (CH); Robert MAH, Basel (CH); Christophe MURA, Basel (CH); Pascal RIGOLLIER, Basel (CH); Anirudh Cadapa PRAHALLAD, Basel (CH); Nadine SCHNEIDER, Basel (CH); Rowan STRINGER, Basel (CH); Stefan STUTZ, Basel (CH); Andrea VAUPEL, Basel (CH); Nicolas WARIN, Basel (CH); Rainer WILCKEN, Basel (CH); Andreas WEISS, Basel (CH)

(21) Appl. No.: 18/572,518

(22) PCT Filed: Jun. 23, 2022
(86) PCT No.: PCT/IB2022/055820

§ 371 Date: Dec. 20, 2023

Related U.S. Application Data
(60) Provisional application No. 63/328,442, filed on Apr. 7, 2022.
(60) Provisional application No. 63/214,001, filed on Jun. 23, 2021.

(51) Int. Cl. A61K 31/416; A61K 31/519; A61K 39/00; A61K 45/06; A61P 35/00; C07K 16/22

(52) U.S. Cl. CPC A61K 31/416; A61K 31/519; A61K 45/06; A61P 35/00; C07K 16/22; A61K 2039/505

(57) ABSTRACT

The present invention relates to a pharmaceutical combination comprising a KRAS G12C inhibitor and one or more therapeutic agents which is selected from an agent targeting the MARK pathway or an agent targeting parallel pathways; and pharmaceutical compositions comprising the same. The invention also relates to KRAS G12C inhibitors alone or said combinations for use in methods of treating a cancer or a tumor, in particular wherein the cancer or tumor is KRAS G12C mutant.

### RELATED APPLICATIONS

The instant application is a 35 U.S.C. § 371 filing of International Patent Application No. PCT/IB2022/055820, filed Jun. 23, 2022, which claims priority to U.S. Provisional Patent Application Serial Nos. 63/328,442, filed Apr. 7, 2022, and 63/214,001, filed Jun. 23, 2021, the entire contents of which are incorporated herein by reference for all purposes.

### SEQUENCE LISTING

The instant application contains a Sequence Listing which has been submitted electronically in ASCII format. Said ASCII copy, created on Jan. 17, 2024, is named 747401_NIT-59141US_ST25.txt, is 2,789 bytes in size is filed herewith and is incorporated herein by reference.

### FIELD OF THE INVENTION

The present invention relates to a KRAS G12C inhibitor and its uses in treating cancer, particularly KRAS G12C mutant cancer (e.g. lung cancer, non-small cell lung cancer, colorectal cancer, pancreatic cancer or a solid tumor) in combination with one or two additional therapeutically active agents. The present invention relates to a pharmaceutical combination comprising (i) a KRAS G12C inhibitor, such as Compound A, or a pharmaceutically acceptable salt thereof, and a second therapeutic agent which is selected from an agent targeting the MAPK pathway or parallel pathways such as the PI3K/AKT pathway. The second therapeutic agent may be selected from an EGFR inhibitor, a SOS inhibitor, a SHP2 inhibitor (such as TN0155, or a pharmaceutically acceptable salt thereof), a Raf-inhibitor, an ERK inhibitor, a MEK inhibitor, AKT inhibitor, a PI3K inhibitor, an mTOR inhibitor, a CDK4/6 inhibitor, an FGFR inhibitor and combinations thereof. The present invention also relates to a triple combination comprising a KRAS G12C inhibitor, such as Compound A, or a pharmaceutically acceptable salt thereof, and a second therapeutic agent which is a SHP2 inhibitor (such as TNO155, or a pharmaceutically acceptable salt thereof) and a third therapeutic agent, optionally wherein the third therapeutic agent may be selected from an EGFR inhibitor, a SOS inhibitor, a Raf-inhibitor, an ERK inhibitor, a MEK inhibitor, AKT inhibitor, a PI3K inhibitor, an mTOR inhibitor, a CDK4/6 inhibitor and an FGFR inhibitor.

The present invention also relates to pharmaceutical compositions comprising the same; and methods of using such combinations and compositions in the treatment or prevention of a cancer or a solid tumor, particularly a KRAS G12C mutant cancer or a KRAS G12C solid tumor.

### BACKGROUND

Cancer growth is driven by many diverse complex mechanisms. Resistance to a given therapy inevitably occurs in some cancers. Inhibiting the MAPK pathway induces feedback mechanisms and pathway rewiring causing its subsequent reactivation. One common mechanism is for example the activation of Receptor Tyrosine Kinases (RTKs).

In addition, despite the recent successes of targeted therapies and immunotherapies, some cancers, in particular, metastatic cancers remain largely incurable.

The KRAS oncoprotein is a GTPase with an essential role as regulator of intracellular signaling pathways, such as the MAPK, PI3K and Ra1 pathways, which are involved in proliferation, cell survival and tumorigenesis. Oncogenic activation of KRAS occurs predominantly through missense mutations in codon 12. KRAS gain-of-function mutations are found in approximately 30% of all human cancers. KRAS G12C mutation is a specific sub-mutation, prevalent in approximately 13% of lung adenocarcinomas, 4% (3-5%) of colon adenocarcinomas and a smaller fraction of other cancer types.

In normal cells, KRAS alternates between inactive GDP-bound and active GTP-bound states. Mutations of KRAS at codon 12, such as G12C, impair GTPase-activating protein (GAP)-stimulated GTP hydrolysis. In that case, the conversion of the GTP to the GDP form of KRAS G12C is therefore very slow. Consequently, KRAS G12C shifts to the active, GTP-bound state, thus driving oncogenic signaling.

CDKN2A, also known as cyclin-dependent kinase inhibitor 2A, is a gene which codes for the INK4 family member p16 (or p16INK4a) and p14arf which act as tumor suppressors by regulating the cell cycle. p16 inhibits cyclin dependent kinases 4 and 6 (CDK4 and CDK6) and thereby activates the retinoblastoma (Rb) family of proteins, which block traversal from G1 to S-phase. p14ARF (known as p19ARF in the mouse) activates the p53 tumor suppressor. CDKN2A is thought to be the second most commonly inactivated gene in cancer after p53.

Mutations in CDKN2A have been described in cancers such as melanoma, gastric lymphoma, Burkitt's lymphoma, head & neck squamous cell carcinoma, oral cancer, pancreatic adenocarcinoma, non-small cell lung carcinoma, esophageal squamous cell carcinoma, gastric cancer, colorectal cancer, epithelial ovarian carcinoma and prostate cancer.

The PIK3CA gene (Phosphatidylinositol-4,5-Bisphosphate 3-Kinase Catalytic Subunit Alpha) is a gene which encodes p110 which is involved in proliferation, growth, differentiation, motility, and survival of cells. A mutation in the PIK3CA gene creates abnormal p110 proteins at an increased rate. The PIK3CA gene mutation has been found in the breast cancer, ovarian cancer, lung cancer, stomach cancer, gastric cancer and brain cancer.

Lung cancer remains the most common cancer type worldwide and the leading cause of cancer deaths in many counties, including the United States. NSCLC accounts for about 85% of all lung cancer diagnoses. KRAS mutations are detected in approximately 25% of patients with lung adenocarcinomas (Sequist et al 2011). They are most commonly seen at codon 12, with KRAS G12C mutations being most common (40% overall) in both adenocarcinoma and squamous NSCLC (Liu et al 2020). The presence of KRAS mutations is prognostic of poor survival and has been associated with reduced responsiveness to EGFR TKI treatment.

Standard of care treatment for patients with KRAS G12C mutant NSCLC consists of platinum-based chemotherapy and immune checkpoint inhibitors. Sotorasib, a KRAS G12C inhibitor, has recently received accelerated approval from the FDA for this indication and for adult patients who have received at least one prior systemic therapy, with further confirmatory trials currently ongoing. Sotorasib received accelerated approval by the US FDA (Food and Drug Administration) in May 2021 and conditional marking authorization by the European Commission (EC) in January 2022 in patients with KRAS G12C-mutated locally advanced or metastatic non-small cell lung cancer (NSCLC). In this patient population in a phase 2 single arm study of 126 patients, sotorasib demonstrated an ORR of 37% (95% CI 28.6-46.2), median DOR of 11.1 months, median PFS of 6.8 months, and median OS of 12.5 months (Skoulidis et al, N Engl J Med; 384:2371-81). Adagrasib, another KRAS G12C inhibitor, is also in clinical development in KRAS G12C-mutated malignancies, with a preliminary ORR of 45% in patients with NSCLC (Janne et al 2019, Presented at AACR-NCI-EORTC International Conference on Molecular Targets, 28 Oct. 2019).

Immunotherapy for NSCLC with immune checkpoint inhibitors has demonstrated promise, with some NSCLC patients experiencing durable disease control for years. However, such long-term non-progressors are uncommon, and treatment strategies that can increase the proportion of patients responding to and achieving lasting remission with therapy are urgently needed.

Colorectal cancer (CRC) is the fourth most frequently diagnosed cancer and the second leading cause of cancer related death in the United States. The number of new cases of CRC was approximately 150,000 in the USA in 2019, whereas more than 300,000 patients are estimated to be diagnosed with CRC in the EU in 2020 (European Cancer Information System 2020). Despite observed improvements in the overall incidence rate of CRC, the incidence in patients younger than 50 years has been increasing in recent years (Bailey et al 2015) with the authors estimating that the incidence rates for colon and rectal cancers may increase by 90% and about 124%, respectively, for patients 20-34 years of age by 2030. Systemic therapy for metastatic CRC includes various agents used alone or in combination, including chemotherapies such as 5-fluorouracil/leucovorin, capecitabine, oxaliplatin, and irinotecan; anti-angiogenic agents such as bevacizumab and ramucirumab; anti-EGFR agents including cetuximab and panitumumab for KRAS/NRAS wild-type cancers; and immunotherapies including nivolumab and pembrolizumab. Despite multiple active therapies, however, metastatic CRC remains incurable. While CRCs that are deficient in mismatch repair (MSI-high) exhibit high response rates to immune checkpoint inhibitor therapy, mismatch repair proficient CRCs do not. Since KRAS-mutant CRCs are typically mismatch repair proficient and are not candidates for anti-EGFR therapy, this subtype of CRC is particularly in need of improved therapies.

Tumor profiling data show that there is a subset of solid tumors other than NSCLC and CRC that harbor KRAS G12C mutations. KRAS G12C is present in approximately 1-2% of malignant solid tumors, including approximately 1% of all pancreatic cancers (Biernacka et al 2016, Zehir et al 2017). KRAS G12C mutations were also found in appendiceal cancer, small-bowel cancer, hepatobiliary cancer, bladder cancer, ovarian cancer and cancers of unknown primary site (Hassar et al, N Engl Med 2021 384;2 185-187).

Several targeted therapies are at present in clinical testing aiming to address patients with KRAS mutations by inhibiting the RAS pathway. However, the benefit of these therapies for tumors harboring KRAS G12C mutations remains uncertain at present, as not all patients responded and in several instances, the duration of the reported responses were short, likely due to the emergence of resistance, mediated at least in part by RAS gene mutations that disrupt inhibitor binding and reactivation of downstream pathways.

Acquired resistance to single-agent therapy eventually occurs in most patients treated with KRAS G12C inhibitors. For example, out of 38 patients included in a study with adagrasib: 27 with non-small-cell lung cancer, 10 with colorectal cancer, and 1 with appendiceal cancer, putative mechanisms of resistance to adagrasib were detected in 17 patients (45% of the cohort), of whom 7 (18% of the cohort) had multiple coincident mechanisms. Acquired KRAS alterations included G12D/R/V/W, G13D, Q61H, R68S, H95D/Q/R, Y96C, and high-level amplification of the KRASG12C allele. Acquired bypass mechanisms of resistance included MET amplification; activating mutations in NRAS, BRAF, MAP2K1, and RET; oncogenic fusions involving ALK, RET, BRAF, RAF1, and FGFR3; and loss-of-function mutations in NF1 and PTEN (Awad et al, Acquired Resistance to KRASG12C Inhibition in Cancer, N Engl J Med 2021; 384:2382-93. Tanaka et al (Cancer Discov 2021; 11:1913-22) describe a novel KRAS Y96D mutation affecting the switch-II pocket, to which adagrasib and other inactive-state KRAS G12C inhibitors bind, which interfered with key protein-drug interactions and conferred resistance to these inhibitors in engineered and patient-derived KRASG12C cancer models.

Additional treatment options to overcome resistance mechanisms that arise during treatment with KRAS inhibitors such as adagrasib or sotorasib are therefore needed.

There thus remains a high unmet medical need for new treatment options for patients suffering from cancer (including advanced and/or metastatic cancer including lung cancer (including NSCLC), colorectal cancer, pancreatic cancer and a solid tumor), especially when the cancer or solid tumor harbors a KRAS G12C mutation. It is also important to provide a potentially beneficial novel therapy for incurable disease, especially for patients with KRAS G12C mutant tumors who have already received and failed standard of care therapy for their indication or are intolerant or ineligible to approved therapies and have therefore limited treatment options.

### BRIEF DESCRIPTION OF THE DRAWINGS

FIGS. 1 to 5 are waterfall plots to represent the efficacy of a KRAS G12C inhibitor alone and in combination with other agents in CRC and lung cancer patient-derived xenograft models. Each Figure shows the response to a particular treatment for each individual mouse model indicated as % best average response (Best Avg. Resp.) on the (vertical) y-axis. Best average response is the minimum average response (the average change in volume over all time points between day 0 and day X—this is similar to cumulative sum or area under the curve. It incorporates the speed, strength, and durability of response into a single value).

FIG. 1A and FIG. 1B: Waterfall plot to show the efficacy of a KRAS G12C inhibitor and in combination with agents targeting the MAPK pathway in CRC patient-derived xenograft models shown as best average response results.

FIG. 2: Waterfall plot to show the efficacy of a KRAS G12C inhibitor and in combination with agents targeting parallel pathways in CRC patient-derived xenograft models shown as best average response results.

FIG. 3A and FIG. 3B: Waterfall plot to show the efficacy of triple combinations comprising a KRAS G12C inhibitor in NSCLC patient-derived xenograft models shown as best average response results.

FIG. 4A and FIG. 4B: Waterfall plot to show the efficacy of a KRAS G12C inhibitor and in combination with agents targeting the MAPK pathway in NSCLC patient-derived xenograft models shown as best average response results.

FIG. 5: Waterfall plot to show the efficacy of a KRAS G12C inhibitor and in combination with agents targeting parallel pathways in NSCLC patient-derived xenograft models shown as best average response results.

FIG. 6: Spider plot to show the % Tumor volume change over time. Fragments of the CRC or lung cancer are implanted in the mice, and when the tumor reached the required volume (T=0, on the x-axis of the spider plots), the control mice models are assigned into groups and the tumor volume monitored.

Spider plots show the % Tumor volume change over time of each tumor model for untreated control post enrolment. Fragments of the CRC or lung cancer were implanted in the mice, and when the tumor reached the required volume (T=0, on the x-axis of the spider plots), the control mice were assigned as controls and the tumor volume monitored.

FIG. 7: Kaplan-Meier time to tumor volume doubling in patient-derived NSCLC and CRC xenografts plots. Combination treatment benefit was observed for time to tumor volume doubling.

FIG. 8: Compound A potently inhibited KRAS G12C cellular signaling and proliferation in a mutant-selective manner and demonstrated dose-dependent antitumor activity, with efficacy driven by daily AUC.

A. Aggregated best tumor growth inhibition in six KRASG12C tumor models. JDQ443 efficacy was evaluated after oral dosing of 10, 30 and 100 mg/kg/day in six human KRAS G12C mutant CDX models in mice. In dark grey NSCLC cell line models are depicted, while in light grey PDAC (MIA Paca-2) and esophageal (KYSE-410) cancer cell line models are shown. Data are means from 2-11 independent in vivo studies. B-G. CDX-bearing mice with KRAS G12C-mutated (C-G) and non-KRAS G12C-mutated (NCI-441, KRASG12V; B) tumors were treated orally with JDQ443 at indicated doses and schedules. G. LU99 tumor-bearing mice were treated with JDQ443 by continuous intravenous infusion using a minipump. H-I. Simulated pop-PKPD metrics (H) daily AUC of JDQ443 in mouse blood and (I) average free KRASG12C levels in tumor at steady state, are correlated with the observed efficacy in LU99 (T/C or % regression). Points correspond to the mean and the error bars to ±1 S.D of the simulated PK/PD metrics based on 100 simulations and observed efficacy metrics.

*p<0.05 vs vehicle, #p<0.05 vs each other, by one-way ANOVA.

FIG. 9: Effect of Compound A (JDQ443), sotorasib (AMG510) and adagrasib (MRTX-849) on the proliferation of KRAS G12C/H95 double mutants. Ba/F3 cells expressing the indicated FLAG-KRAS^{G12C} single or double mutants were treated with the indicated compound concentrations for 3 days and the inhibition of proliferation was assessed by Cell titer glo viability assay. The y-axis shows the % growth of treated cells relative to day 3 treatment, the x-axis shows the log concentration in M of the KRASG12C inhibitor.

FIG. 10: Western blot analysis of ERK phosphorylation to assess the effect of Compound A (JDQ443), sotorasib (AMG510) and adagrasib (MRTX-849) on the signaling of KRAS G12C/H95 double mutants. Ba/F3 cells expressing the indicated FLAG-KRAS^{G12C} single or double mutants were treated with the indicated compound concentrations for 30 min and the inhibition of the MAPK pathway was assessed by probing the cell lysates for reduction of pERK by westernblot. FIG. 11A and FIG. 11B: Synergy scores (SS) obtained in 3-day cell viability assays in NCI H23 cells. Matrix combination proliferation assays (treatment time 3 days, cell titer glow assay) were performed with a KRAS^{G12C} inhibitor (labelled “KRAS^{G12C}i” in FIG. 11) as single agent or in combination with 10 μM SHP099, a SHP2 inhibitor, (labelled “SHP2i” in FIG. 11) in the presence of either upstream receptor kinase inhibitors BGJ398, an FGFR inhibitor (labelled “FGFRi” in FIG. 11), and erlotinib, an EGFR inhibitor (labelled “EGFRi” in FIG. 11) or trametinib, a MEK inhibitor (labelled as “MEKi” in FIG. 11) or the PI3K effector arm inhibitors alpelisib (labelled “PI3Kαi” in FIG. 11) and GDC0941, a pan-PI3K inhibitor (labelled “panPI3Ki” in FIG. 11) in a KRAS G12C mutated H23 cell line. Synergy scores (SS) are indicated as “SS” values on top of each grid. Values in the grid are growth inhibition (%) values: a value higher than 100% indicates cell death. The values on the x-axis of each grid indicate the concentration (in M) of the KRASG12c inhibitor used. The values on the y-axis of each grid shows the concentration (in M) of the second agent (i.e the FGFR inhibitor, the EGFR inhibitor, the MEK inhibitor, the PI3αK inhibitor and the pan-PI3K inhibitor respectively).

FIG. 12: PI3K+/−CDK4 inhibition improves KRASG12C+SHP2 combination treatment. Double and higher order combinations of Compound A (JDQ443) improve single-agent activity in LU99 lung xenografts (KRAS G12C, PIK3CAmut, CDKN2Adel). Compound A in combination with a SHP2 inhibitor, PI3K inhibitor or CDK4/6 inhibitor delays time to progression (TTP) compared to single agent treatment with Compound A. The time to progression increased from the single agent to the quadruple combination (TTP: single agent<double combination<triple combination<quadruple combination).

FIG. 13: Dose response of Compound A (JDQ443) in combination with an EGFR inhibitor in NSCLC cell lines and CRC cell lines.

FIG. 14: In vitro viability of the colorectal cancer cell lines and lung cancer was assessed using the CellTiterGlo following 7-day treatment with the KRASG12C inhibitor Compound A (“NVP-JDQ443” in FIG. 14) combined with the SOS1 inhibitor BI-3406. Growth inhibition %: 0-99=delayed proliferation, 100=growth arrest/stasis, 101-200=reduction in cell number/cell death.

FIG. 15: PK and target occupancy profiles of JDQ443 RD 200 mg BID. Top panel shows the PK profile at steady state. Error bars indicate standard deviation for PK profile at each timepoint. The bottom panel shows the predicted target occupancy profile, where the line shows the simulated median and the shaded area shows the 5-95 percentile prediction interval.

FIG. 16: The top panel shows the best overall response across dose levels and indications for JDQ443 monotherapy. Waterfall plot: 37 (94.9%) patients with available change from baseline tumor assessments; data are plotted out of N=39 JDQ443 single-agent patients. Best overall responses are investigator assessed per RECIST v1.1. Three (7.7%) patients had a uPR, which contributed toward the ORR (confirmed and unconfirmed). uPR=unconfirmed PR pending confirmation, treatment ongoing with no PD. Intra-patient dose escalation, per protocol, occurred in four patients from 200 mg QD to 200 mg BID.

The bottom panel shows best overall responses across dose in all patients with NSCLC. Waterfall plot: 19 (95.0%) NSCLC patients with available change from baseline tumor assessments; data are plotted out of N=20 NSCLC patients in JDQ443 single-agent cohorts.

FIG. 17: PET scans showing a substantial reduction in the 2-[fluorine-18]-fluoro-2-deoxy-d-glucose (18-F-FDG) avidity of the tumor mass after four cycles of treatment with Compound A administered at 200 mg BID to a patient with NSCLC. CT: computerized tomography; PET, positron emission tomography. Arrows indicate sites of tumor.

FIG. 18: Serial axial CT/PET images and steady-state (cycle 1 day 14) JDQ443 PK exposures for combination therapy with Compound A. The combination of Compound A and a SHP2 inhibitor is efficacious. Efficacy of Compound A and TNO155 in a patient with duodenal papillary cancer. Arrows indicate sites of tumor.

### SUMMARY

The invention provides new treatment options for patients suffering from cancer (including advanced and/or metastatic cancer and seeks particularly to improve outcomes for patients with KRAS G12C-driven cancers.

Provided herein are compounds, and combinations of compounds, and their uses in methods of treating cancer including lung cancer (including NSCLC), colorectal cancer, pancreatic cancer and a solid tumor), especially when the cancer or solid tumor harbors a KRAS G12C mutation. The present invention also provides a potentially beneficial novel therapy for incurable disease, especially for patients with KRAS G12C mutated tumors who have already received and failed standard of care therapy for their indication or are intolerant or ineligible to approved therapies and have therefore limited treatment options.

In addition, the present invention also provides Compound A alone or in combination with one or more additional therapeutic agents for use in a method of treatment for cancer patients who have developed resistance to other therapies, such as prior treatment with other KRAS inhibitors such as adagrasib and sotorasib; more preferably prior treatment with sotorasib.

Compound A is a selective covalent irreversible inhibitor of KRAS G12C which exhibits a novel binding mode, exploiting unique interactions with KRASG12C. Notably, Compound A traps KRAS G12C in a GDP-bound, inactive state while avoiding direct interaction with H95, a recognized route for resistance (Awad M M, et al. New Engl J Med 2021; 384:2382-2392). Compound A potently inhibited KRAS G12C H95Q, a double mutant mediating resistance to adagrasib in clinical trials.

Compound A demonstrates potent anti-tumor activity and favorable pharmacokinetic properties in preclinical models. Compound A is orally bioavailable, achieves exposures in a range predicted to confer anti-tumor activity, and is well-tolerated.

Preliminary data (Phase 1b) from the KontRASt-01 study (NCT04699188) showed that Compound A, a selective, covalent, and orally bioavailable KRASG12C inhibitor, demonstrated anti-tumor activity, high systemic exposure at its recommended dose, and a favorable safety profile based on initial clinical data in patients with KRAS G12C-mutated solid tumors

KRAS G12C inhibitors are specifically designed to inhibits KRAS G12C. However, many tumors have KRAS WT, HRAS and NRAS proteins which are not inhibited by KRAS G12C inhibitors. Upon KRAS G12C inhibitor treatment, reactivated RTKs for instance can feed via these proteins into the MAPK pathway, thus counteracting anti-tumor activity. Likewise, many RTKs as well as RAS proteins directly activate parallel pathways, e.g. the PI3K/AKT pathway.

The data and the Examples herein show that the addition of another therapeutically active agent which targets the MAPK pathway or parallel pathways, e.g. the PI3K/AKT pathway, to a KRAS G12C inhibitor in a combination therapy has the potential to increase the depth and durability of anti-tumor response.

For example, inhibitors of SHP2 have the potential to synergize with a KRAS G12C inhibitor such as Compound A. Inhibition of SHP2 inhibits growth of KRAS-mutant cancer cell lines in part by shifting the pool of KRAS to the inactive GDP-loaded state. As Compound A binds exclusively to GDP-bound KRASG12C, combined SHP2 and KRASG12C inhibition is predicted to be synergistic due to the increased target pool for irreversible Compound A binding.

As seen in the Examples, highest synergy scores were obtained in the presence of a PI3K inhibitor in combination with a KRAS G12C inhibitor alone or in the presence of a SHP2 inhibitor in a cell viability assay. Thus the present invention also provides triple or quadruple combinations as described herein.

As seen in the Examples, Compound A, a KRAS G12C inhibitor, showed deep tumor in xenograft models, in particular in cancer xenograft models harboring one or more mutations selected from KRAS G12C, PIK3CA and CDKN2A. The anti-tumor response of a KRAS G12C inhibitor as single agent was improved with each of the combination partners tested, with some tumors even regressing with the combination treatment. Triple combinations and quadruple combinations appeared to improve the response further.

In summary, it can be seen that Compound A with its unique properties and tolerability and safety profile may be especially useful to treat cancer and in particular the cancers described herein, alone or in combination with one or more (e.g. one, two or three) therapeutic agents as described herein.

In particular, combinations of a KRAS G12C inhibitor (such as Compound A) with other inhibitors of MAPK pathway or inhibitors of PI3K/AKT pathway have the potential to further enhance anti-tumor response and overcome potential resistance. Such combination therapies may be useful in treating cancer, in particular, cancers driven by KRAS G12C mutations. The second therapeutic agent may be selected from an EGFR inhibitor, a SOS inhibitor, a SHP2 inhibitor (such as TNO155, or a pharmaceutically acceptable salt thereof), a Raf-inhibitor, an ERK inhibitor, a MEK inhibitor, AKT inhibitor, a PI3K inhibitor, an mTOR inhibitor, a CDK4/6 inhibitor and combinations thereof.

The combinations and methods of the present invention may thus also provide clinical benefit in patients that have for instance acquired resistance to KRAS G12C inhibitor by reactivation of RTK-MAPK pathway bypassing KRAS G12C to signal through WT KRAS, NRAS and/or HRAS. In addition, inhibition of EGFR targets the KRAS signaling pathway upstream of KRAS and may enhance the anti-tumor activity of a KRAS G12C inhibitor such as Compound A in KRAS G12C mutant cancer. Cancers to be treated by the combinations and methods of the present invention include a cancer or solid tumor which harbors one, two or three mutations selected from KRAS G12C, PIK3CA and CDKN2A, and combinations thereof; for example, a cancer harboring KRAS G12C and CDKN2A mutations; and a cancer harboring KRAS G12C, PIK3CA and CDKN2A mutations.

The present invention therefore also provides a pharmaceutical combination comprising a KRAS G12C inhibitor, such as Compound A, or a pharmaceutically acceptable salt thereof, and at least one additional therapeutically active agent. The additional therapeutically active agent may be an agent targeting the MAPK pathway or an agent targeting parallel pathways.

The present invention therefore also provides a pharmaceutical combination comprising a KRAS G12C inhibitor, such as Compound A, or a pharmaceutically acceptable salt thereof, and a therapeutically active agent which is selected from the group consisting of an EGFR inhibitor, a SOS inhibitor, a SHP2 inhibitor (such as TNO155, or a pharmaceutically acceptable salt thereof), a Raf-inhibitor, an ERK inhibitor, a MEK inhibitor, AKT inhibitor, a PI3K inhibitor, an mTOR inhibitor, a CDK4/6 inhibitor and combinations thereof.

The present invention therefore also provides a pharmaceutical combination comprising a KRAS G12C inhibitor, such as Compound A, or a pharmaceutically acceptable salt thereof, a SHP2 inhibitor (such as TNO155, or a pharmaceutically acceptable salt thereof) and another therapeutically active agent which is selected from the group consisting of an EGFR inhibitor (such as cetuximab, panitumab, afatinib, lapatinib, erlotinib, gefitinib, osimertinib or nazartinib), a SOS inhibitor (such as BAY-293, BI-3406, or BI-1701963), a Raf-inhibitor (e.g. belvarafenib or LXH254 (naporafenib)), an ERK inhibitor (such as LTT462 (rineterkib), GDC-0994, KO-947, Vtx-11e, SCH-772984, MK2853, LY3214996 or ulixertinib), a MEK inhibitor (such as pimasertib, PD-0325901, selumetinib, trametinib, binimetinib or cobimetinib), AKT inhibitor (such as capivasertib (AZD5363) or ipatasertib), a PI3K inhibitor (such as AMG 511, buparlisib, alpelisib), an mTOR inhibitor (such as everolimus or temsirolimus), and a CDK4/6 inhibitor (such as ribociclib, palbociclib or alemaciclib).

The present invention also provides a pharmaceutical combination comprising 1-{6-[(4M)-4-(5-Chloro-6-methyl-1H-indazol-4-yl)-5-methyl-3-(1-methyl-1H-indazol-5-yl)-1H-pyrazol-1-yl]-2-azaspiro[3.3]heptan-2-yl}prop-2-en-1-one, having the structure

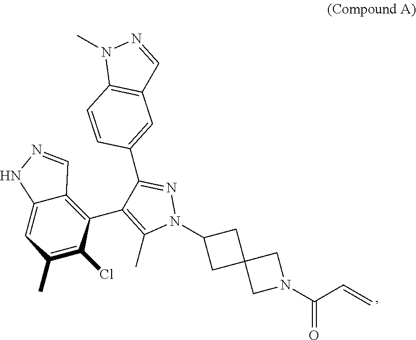

or a pharmaceutically acceptable salt thereof,
and a second therapeutically active agent which is selected from an EGFR inhibitor (such as cetuximab, panitumumab, erlotinib, gefitinib, osimertinib or nazartinib), a SOS inhibitor (such as BAY-293, BI-3406, or BI-1701963), a Raf-inhibitor (e.g. belvarafenib or LXH254 (naporafenib)), an ERK inhibitor (such as LTT462 (rineterkib), GDC-0994, KO-947, Vtx-11e, SCH-772984, MK2853, LY3214996 or ulixertinib), a MEK inhibitor (such as pimasertib, PD-0325901, selumetinib, trametinib, binimetinib or cobimetinib), an AKT inhibitor (such as capivasertib (AZD5363) or ipatasertib), a PI3K inhibitor (such as AMG 511, buparlisib, alpelisib), an mTOR inhibitor (such as everolimus or temsirolimus), and a CDK4/6 inhibitor (such as ribociclib, palbociclib or alemaciclib).

The present invention also provides a pharmaceutical combination comprising Compound A, or a pharmaceutically acceptable salt thereof, and a second therapeutically active agent which is selected from a Raf-inhibitor (e.g. belvarafenib or LXH254 (naporafenib)), an ERK inhibitor (such as LTT462 (rineterkib), GDC-0994, KO-947, Vtx-11e, SCH-772984, MK2853, LY3214996 or ulixertinib), a MEK inhibitor (such as pimasertib, PD-0325901, selumetinib, trametinib, binimetinib or cobimetinib), a PI3K inhibitor (such as AMG 511, buparlisib, alpelisib), an mTOR inhibitor (such as everolimus or temsirolimus), and a CDK4/6 inhibitor (such as ribociclib, palbociclib or alemaciclib).

In embodiments of the invention, the second therapeutically active agent may be selected from an FGFR inhibitor such as infigratinib (BGJ398), pemigatinib, erdafitinib, derazantinib; and futibatinib. The present invention also provides a pharmaceutical combination comprising (a) Compound A, or a pharmaceutically acceptable salt thereof, (b) a SHP2 inhibitor (such as TNO 155, or a pharmaceutically acceptable salt thereof), and (c) a third therapeutically active agent which is selected from a Raf-inhibitor (e.g. belvarafenib or LXH254 (naporafenib)), an ERK inhibitor (such as LTT462 (rineterkib), GDC-0994, KO-947, Vtx-11e, SCH-772984, MK2853, LY3214996 or ulixertinib), a MEK inhibitor (such as pimasertib, PD-0325901, selumetinib, trametinib, binimetinib or cobimetinib), a PI3K inhibitor (such as AMG 511, buparlisib, alpelisib), an mTOR inhibitor (such as everolimus or temsirolimus), and a CDK4/6 inhibitor (such as ribociclib, palbociclib or alemaciclib).

In embodiments of the invention, the third therapeutically active agent may be selected from an FGFR inhibitor such as infigratinib (BGJ398), pemigatinib, erdafitinib, derazantinib; and futibatinib.

The present invention also provides a pharmaceutical combination comprising (a) Compound A, or a pharmaceutically acceptable salt thereof, (b) TNO 155, or a pharmaceutically acceptable salt thereof,

and (c) a third therapeutically active agent which is selected from a Raf-inhibitor (e.g. belvarafenib or LXH254 (naporafenib)), an ERK inhibitor (such as LTT462 (rineterkib), GDC-0994, KO-947, Vtx-11e, SCH-772984, MK2853, LY3214996 or ulixertinib), a MEK inhibitor (such as pimasertib, PD-0325901, selumetinib, trametinib, binimetinib or cobimetinib), a PI3K inhibitor (such as AMG 511, buparlisib, alpelisib), an mTOR inhibitor (such as everolimus or temsirolimus), and a CDK4/6 inhibitor (such as ribociclib, palbociclib or alemaciclib).

The present invention also provides a combination of the invention comprising Compound A, or a pharmaceutically acceptable salt thereof, and a second agent which is selected from:
(i) LXH254 (naporafenib), or a pharmaceutically acceptable salt thereof;
(ii) trametinib, pharmaceutically acceptable salt or solvate thereof, e.g. the DMSO solvate thereof;
(iii) LTT462 (rineterkib), or a pharmaceutically acceptable salt thereof, e.g. the HCl salt thereof;
(iv) BYL719 (alpelisib), or a pharmaceutically acceptable salt thereof;
(v) LEE011 or a pharmaceutically acceptable salt thereof, e.g. the succinate salt thereof; and
(vi) everolimus (RAD001).
or a pharmaceutically acceptable salt thereof.

The present invention also provides a combination of the invention comprising (a) Compound A, or a pharmaceutically acceptable salt thereof, (b) TNO 155, or a pharmaceutically acceptable salt thereof, and a third agent which is selected from:
(i) naporafenib (LXH254), or a pharmaceutically acceptable salt thereof;
(ii) trametinib, pharmaceutically acceptable salt or solvate thereof, e.g. the DMSO solvate thereof;
(iii) rineterkib (LTT462), or a pharmaceutically acceptable salt thereof, e.g. the HCl salt thereof;
(iv) alpelisib (BYL719), or a pharmaceutically acceptable salt thereof;
(v) ribociclib (LEE011), or a pharmaceutically acceptable salt thereof, e.g. the succinate salt thereof; and
(vi) everolimus (RAD001).
or a pharmaceutically acceptable salt thereof.

It will be understood that reference herein to “a combination of the invention” or “the combination(s) of the invention” is intended to include each of these pharmaceutical combinations individually and to all of these combinations as a group.

In particular, reference to “a combination of the invention” is intended to include a combination of a KRASG12C inhibitor and a SHP2 inhibitor (e.g. Compound A and TN0155); a combination of a KRASG12C inhibitor and a PI3K inhibitor (e.g. Compound A and alpelisib (BYL719)); a KRASG12C inhibitor and a CDK4/6 inhibitor (e.g. Compound A and ribociclib).

Triple combinations are also included in the definition of “a combination of the invention”. Preferred embodiments include (i) a combination of Compound A, TNO155 and alpelisib and (ii) a combination of Compound A, TNO155 and ribociclib.

The present invention provides these pharmaceutical combinations for use in treating a cancer as described herein.

Efficacy of the therapeutic methods of the invention may be determined by methods well known in the art, e.g. determining Best Overall Response (BOR), Overall Response Rate (ORR), Duration of Response (DOR), Disease Control Rate (DCR), Progression Free Survival, (PFS) and Overall Survival (OS) per RECIST v.1.1. The present invention therefore provides a pharmaceutical combination of the invention which improves KRAS G12C inhibitor therapy, e.g. as measured by an increase in one or more of Best Overall Response (BOR), Overall Response Rate (ORR), Duration of Response (DOR), Disease Control Rate (DCR), Progression Free Survival, (PFS) and Overall Survival (OS) per RECIST v.1.1.

In another embodiment of the combination of the invention, Compound A, or a pharmaceutically acceptable salt thereof, the second therapeutically active agent, and the third therapeutically active agent (if present), are in separate formulations.

In another embodiment, the combination of the invention is for simultaneous or sequential (in any order) administration.

In another embodiment is a method for treating or preventing cancer in a subject in need thereof comprising administering to the subject a therapeutically effective amount of the combination of the invention.

In embodiments of the invention, the cancer or tumor to be treated is selected from the group consisting of lung cancer (including lung adenocarcinoma, non-small cell lung cancer and squamous cell lung cancer), colorectal cancer (including colorectal adenocarcinoma), pancreatic cancer (including pancreatic adenocarcinoma), uterine cancer (including uterine endometrial cancer), rectal cancer (including rectal adenocarcinoma), appendiceal cancer, small-bowel cancer, esophageal cancer, hepatobiliary cancer (including liver cancer and bile duct carcinoma), bladder cancer, ovarian cancer and a solid tumor, particularly when the cancer or tumor harbors a KRAS G12C mutation.

In embodiments of the invention, the cancer or tumor to be treated is selected from the group consisting of lung cancer (including lung adenocarcinoma, non-small cell lung cancer and squamous cell lung cancer), colorectal cancer (including colorectal adenocarcinoma), pancreatic cancer (including pancreatic adenocarcinoma), uterine cancer (including uterine endometrial cancer), rectal cancer (including rectal adenocarcinoma), appendiceal cancer, small-bowel cancer, esophageal cancer, hepatobiliary cancer (including liver cancer, bile duct cancer and bile duct carcinoma), bladder cancer, ovarian cancer, duodenal papillary cancer and a solid tumor, particularly when the cancer or tumor harbors a KRAS G12C mutation.

In embodiments of the invention, the cancer or tumor to be treated is selected from non-small cell lung cancer, colorectal cancer, bile duct cancer, ovarian cancer, duodenal papillary cancer and pancreatic cancer.

Cancers of unknown primary site but showing a KRAS G12C mutation may also benefit from treatment with the methods of the invention.

In embodiments of the methods of the invention, the cancer is selected from non-small cell lung cancer, colorectal cancer, pancreatic cancer and a solid tumor.

In a further embodiment of the methods, the cancer is a solid tumor.

In a further embodiment of the methods, the cancer is colorectal cancer.

In a further embodiment of the methods, the cancer is non-small cell lung cancer.

In a further embodiment of the methods, the cancer is pancreatic cancer.

In a further embodiment of the methods, the cancer is a solid tumor.

In a further embodiment of the methods, the cancer is appendiceal cancer.

In a further embodiment of the methods, the cancer is small-bowel cancer.

In a further embodiment of the methods, the cancer is esophageal cancer.

In a further embodiment of the methods, the cancer is hepatobiliary cancer.

In a further embodiment of the methods, the cancer is bladder cancer.

In a further embodiment of the methods, the cancer is ovarian cancer.

In a further embodiment of the methods, the cancer is bile duct cancer.

In a further embodiment of the methods, the cancer is duodenal papillary cancer.

In a further embodiment, the invention provides a combination of the invention for use in the manufacture of a medicament for treating a cancer selected from: non-small cell lung cancer, colorectal cancer, pancreatic cancer and a solid tumor, optionally wherein the cancer or solid tumor is KRAS G12C mutated. In another embodiment is a pharmaceutical composition comprising the combination of the invention.

In a further embodiment, the pharmaceutical composition further comprises one or more pharmaceutically acceptable excipients as described herein.

### KRAS G12C Inhibitors

Examples of KRAS G12C inhibitors useful in combinations and methods of the present invention include Compound A, sotorasib (Amgen), adagrasib (Mirati), D-1553 (InventisBio), BI1701963 (Boehringer), GDC6036 (Roche), JNJ74699157 (J&J), X-Chem KRAS (X-Chem), LY3537982 (Lilly), BI1823911 (Boehringer), AS KRAS G12C (Ascentage Pharma), SF KRAS G12C (Sanofi), RMC032 (Revolution Medicine), JAB-21822 (Jacobio Pharmaceuticals), AST-KRAS G12C (Allist Pharmaceuticals), AZ KRAS G12C (Astra Zeneca), NYU-12VC1 (New York University), and RMC6291 (Revolution Medicines), or a pharmaceutically acceptable salt thereof.

A KRAS G12C inhibitor also includes a compound detailed in A “KRASG12C inhibitor” is a compound selected from the compounds detailed in WO2013/155223, WO2014/143659, WO2014/152588, WO2014/160200, WO2015/054572, WO2016/044772, WO2016/049524, WO2016164675, WO2016168540, WO2017/058805, WO2017015562, WO2017058728, WO2017058768, WO2017058792, WO2017058805, WO2017058807, WO2017058902, WO2017058915, WO2017087528, WO2017100546, WO2017/201161, WO2018/064510, WO2018/068017, WO2018/119183, WO2018/217651, WO2018/140512, WO2018/140513, WO2018/140514, WO2018/140598, WO2018/140599, WO2018/140600, WO2018/143315, WO2018/206539, WO2018/218070, WO2018/218071, WO2019/051291, WO2019/099524, WO2019/110751, WO2019/141250, WO2019/150305, WO2019/155399, WO2019/213516, WO2019/213526, WO2019/217307 and WO2019/217691. Examples are: 1-(4-(6-chloro-8-fluoro-7-(3-hydroxy-5-vinylphenyl)quinazolin-4-yl)piperazin-1-yl)prop-2-en-1-one-methane (1/2) (compound 1); (S)-1-(4-(6-chloro-8-fluoro-7-(2-fluoro-6-hydroxyphenyl)quinazolin-4-yl)piperazin-1-yl)prop-2-en-1-one (compound 2); and 2-((S)-1-acryloyl-4-(2-(((S)-1-methylpyrrolidin-2-yl)methoxy)-7-(naphthalen-1-yl)-5,6,7,8-tetrahydropyrido[3,4-d]pyrimidin-4-yl)piperazin-2-yl)acetonitrile (compound 3).

KRAS G12C inhibitor Compound A A preferred KRAS G12C inhibitor of the present invention is Compound A is 1-{6-[(4M)-4-(5-Chloro-6-methyl-1H-indazol-4-yl)-5-methyl-3-(1-methyl-1H-indazol-5-yl)-1H-pyrazol-1-yl]-2-azaspiro[3.3]heptan-2-yl}prop-2-en-1-one, or a pharmaceutically acceptable salt thereof. Compound A is also known by the name “a(R)-1-(6-(4-(5-chloro-6-methyl-1H-indazol-4-yl)-5-methyl-3-(1-methyl-1H-indazol-5-yl)-1H-pyrazol-1-yl)-2-azaspiro[3.3]heptan-2-yl)prop-2-en-1-one”.

The synthesis of Compound A is described in the Examples below or in Example 1 of PCT application WO2021/124222, published 24 Jun. 2021. Uses of Compound A, alone or in combination with an additional therapeutic agent are described in PCT/CN2021/139694, filed on Dec. 20, 2021. Compound A is also known as “JDQ443” or “NVP-JDQ443”.

The structure of Compound A is as follows:

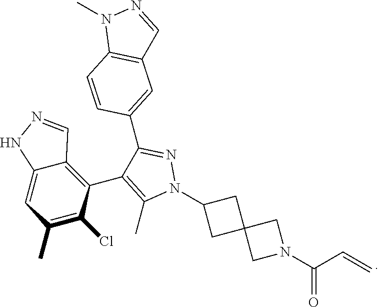

Alternatively, the structure of Compound A may be drawn as follows:

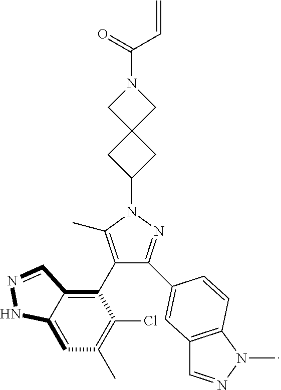

Compound A is a potent and selective KRAS G12C small molecule inhibitor that covalently binds to mutant Cys12, trapping KRAS G12C in the inactive GDP-bound state. Compound A is structurally unique compared with sotorasib or adagrasib; its binding mode is a novel way to reach residue C12 and has no direct interaction with residue H95.

Preclinical data indicate that Compound A binds to KRAS G12C with low reversible binding affinity to the RAS SWII pocket, inhibiting downstream cellular signaling and proliferation specifically in KRAS G12C-driven cell lines but not KRAS wild-type (WT) or MEK Q56P mutant cell lines. Compound A showed deep and sustained target occupancy resulting in anti-tumor activity in different KRAS G12C mutant xenograft models.

### SHP2 Inhibitors

Examples of SHP2 inhibitors useful in combinations and methods of the present invention include TNO155, JAB3068 (Jacobio), JAB3312 (Jacobio), RLY1971 (Roche), SAR442720 (Sanofi), RMC4450 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HB12376 (HUYA Bioscience), ETS001 (Shanghai ETERN Biopharma), TAS-ASTX (Taiho Oncology) and X-37-SHP2 (X-37), or a pharmaceutically acceptable salt thereof.

Examples of SHP2 inhibitors useful in combinations and methods of the present invention, specially in the dual combinations and methods of using the dual combination to treat cancer as described herein, include JAB3068 (Jacobio), JAB3312 (Jacobio), RLY1971 (Roche), SAR442720 (Sanofi), RMC4450 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HB12376 (HUYA Bioscience), ETS001 (Shanghai ETERN Biopharma), TAS-ASTX (Taiho Oncology) and X-37-SHP2 (X-37).

A particularly preferred SHP2 inhibitor for use according to the invention, and especially in the triple combinations of the invention, and methods of using the triple combination may be selected from:

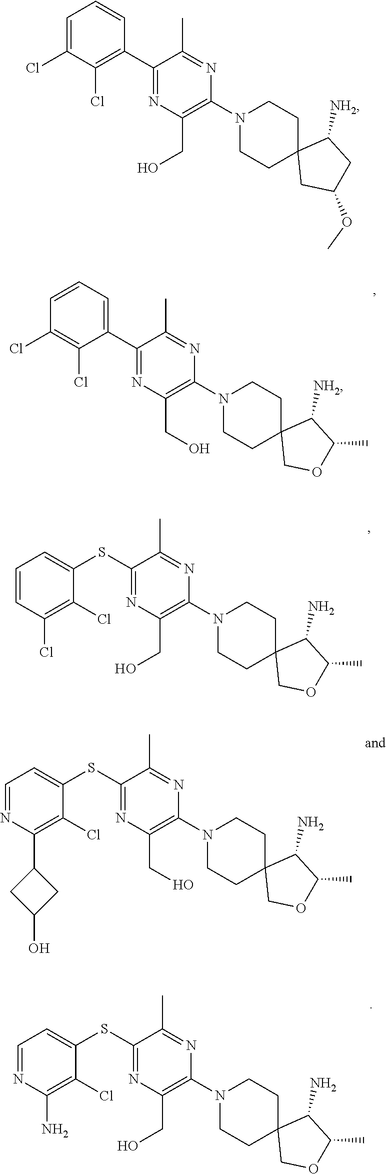 [SMILES: CO[C@H]1C[C@@H](N)C2(CCN(c3nc(C)c(-c4cccc(Cl)c4Cl)nc3CO)CC2)C1 ; Cc1nc(N2CCC3(CC2)CO[C@@H](C)[C@H]3N)c(CO)nc1-c1cccc(Cl)c1Cl ; Cc1nc(N2CCC3(CC2)CO[C@@H](C)[C@H]3N)c(CO)nc1Sc1cccc(Cl)c1Cl ; Cc1nc(N2CCC3(CC2)CO[C@@H](C)[C@H]3N)c(CO)nc1Sc1ccnc(C2CC(O)C2)c1Cl ; Cc1nc(N2CCC3(CC2)CO[C@@H](C)[C@H]3N)c(CO)nc1Sc1ccnc(N)c1Cl]

A particularly preferred SHP2 inhibitor for use according to the invention, and especially in the triple combinations of the invention, and methods of using the triple combination, is (3S,4S)-8-(6-amino-5-((2-amino-3-chloropyridin-4-yl)thio)pyrazin-2-yl)-3-methyl-2-oxa-8-azaspiro[4.5]decan-4-amine (TNO155), or a pharmaceutically acceptable salt thereof. TNO155 is synthesized according to example 69 of WO2015/107495, which is incorporated by reference in its entirety. A preferred salt of TNO155 is the succinate salt.

In addition, SHP2 inhibitors include compounds described in WO2015/107493, WO2015/107494, WO2015/107495, WO2016/203406, WO2016/203404, WO2016/203405, WO2017/216706, WO2017/156397, WO2020/063760, WO2018/172984, WO2017/211303, WO21/061706, WO2019/183367, WO2019/183364, WO2019/165073, WO2019/067843, WO2018/218133, WO2018/081091, WO2018/057884, WO2020/247643, WO2020/076723, WO2019/199792, WO2019/118909, WO2019/075265, WO2019/051084, WO2018/136265, WO2018/136264, WO2018/013597, WO2020/033828, WO2019/213318, WO2019/158019, WO2021/088945, WO2020/081848, WO21/018287, WO2020/094018, WO2021/033153, WO2020/022323, WO2020/177653, WO2021/073439, WO2020/156243, WO2020/156242, WO2020/249079, WO2020/033286, WO2021/061515, WO2019/182960, WO2020/094104, WO2020/210384, WO2020/181283, WO2021/043077, WO2021/028362, WO2020/259679, WO2020/108590 & WO2019/051469.

TNO155 is an orally bioavailable, allosteric inhibitor of Src homology-2 domain containing protein tyrosine phosphatase-2 (SHP2, encoded by the PTPN11 gene), which transduces signals from activated receptor tyrosine kinases (RTKs) to downstream pathways, including the mitogen-activated protein kinase (MAPK) pathway. SHP2 has also been implicated in immune checkpoint and cytokine receptor signaling. TNO155 has demonstrated efficacy in a wide range of RTK-dependent human cancer cell lines and in vivo tumor xenografts.

### PI3K Inhibitors

Examples of PI3K inhibitors useful in the combinations and methods of the present invention include dactolisib, apitolisib, gedatolisib buparlisib, duvelisib, copanlisib, idelalisib, alpelisib taselisib and pictilisib. Preferred PI3K inhibitors of the invention include AMG 511, buparlisib and alpelisib. In preferred embodiments of the invention, alpelisib is the PI3K inhibitor.

In combinations of the invention, each of the therapeutically active agents can be administered separately, simultaneously or sequentially, in any order.

In combinations of the invention, Compound A and/or TNO155 may be administered in an oral dose form.

In another embodiment, there is provided a pharmaceutical composition comprising a pharmaceutical combination of the invention and at least one pharmaceutically acceptable carrier.

### Cancers to be Treated by the Combinations and Methods of the Invention

The combinations of the invention may thus be useful in the treatment of cancer and in cancers or tumors which are KRAS G12C mutated. Combinations of the invention may be useful in the treatment of a cancer or tumor which is selected from the group consisting of lung cancer (including lung adenocarcinoma, non-small cell lung cancer and squamous cell lung cancer), colorectal cancer (including colorectal adenocarcinoma), pancreatic cancer (including pancreatic adenocarcinoma), uterine cancer (including uterine endometrial cancer), rectal cancer (including rectal adenocarcinoma), appendiceal cancer, small-bowel cancer, esophageal cancer, hepatobiliary cancer (including liver cancer and bile duct carcinoma), bladder cancer, ovarian cancer and a solid tumor, particularly when the cancer or tumor harbors a KRAS G12C mutation. Cancers of unknown primary site but showing a KRAS G12C mutation may also benefit from treatment with the methods of the invention.

The cancer or tumor to be treated may be selected from the group consisting of lung cancer (including lung adenocarcinoma, non-small cell lung cancer and squamous cell lung cancer), colorectal cancer (including colorectal adenocarcinoma), pancreatic cancer (including pancreatic adenocarcinoma), uterine cancer (including uterine endometrial cancer), rectal cancer (including rectal adenocarcinoma), appendiceal cancer, small-bowel cancer, esophageal cancer, hepatobiliary cancer (including liver cancer, bile duct cancer and bile duct carcinoma), bladder cancer, ovarian cancer, duodenal papillary cancer and a solid tumor, particularly when the cancer or tumor harbors a KRAS G12C mutation.

The cancer or tumor to be treated may be selected from non-small cell lung cancer, colorectal cancer, bile duct cancer, ovarian cancer, duodenal papillary cancer and pancreatic cancer, particularly when the cancer or tumor harbors a KRAS G12C mutation.

Other cancers to be treated by the compounds, combinations and methods of the invention include gastric cancer, nasopharyngeal cancer, hepatocellular cancer, and Hodgkin's Lymphoma, particularly when the cancer harbors a KRAS G12C mutation.

In particular, the present invention provides methods of treating and combinations for use in treating a cancer which is selected from the group consisting of lung cancer (such as lung adenocarcinoma and non-small cell lung cancer), colorectal cancer (including colorectal adenocarcinoma), pancreatic cancer (including pancreatic adenocarcinoma), uterine cancer (including uterine endometrial cancer), rectal cancer (including rectal adenocarcinoma) and a solid tumor, particularly when the cancer or tumor harbors a KRAS G12C mutation.

As shown in the Examples, Compound A and combinations of the invention have shown anti-tumor activity in xenograft models harboring one, two or three mutations selected from KRAS G12C, PIK3CA and CDKN2A. Therefore, cancers to be treated by the combinations and methods of the present invention include a cancer or solid tumor which harbors one, two or three mutations selected from KRAS G12C, PIK3CA and CDKN2A, and combinations thereof, such as a cancer harboring KRAS G12C and CDKN2A mutations; and a cancer harboring KRAS G12C, PIK3CA and CDKN2A mutations. For example, the cancer to be treated may be lung cancer, (e.g. non-small cell lung cancer) harboring KRAS G12C and CDKN2A mutations; or lung cancer, (e.g. non-small cell lung cancer) KRAS G12C, PIK3CA and CDKN2A mutations.

A cancer which harbors one, two or three mutations selected from KRAS G12C, PIK3CA and CDKN2A may also be selected from the group consisting of lung cancer (including lung adenocarcinoma, non-small cell lung cancer and squamous cell lung cancer), colorectal cancer (including colorectal adenocarcinoma), pancreatic cancer (including pancreatic adenocarcinoma), uterine cancer (including uterine endometrial cancer), rectal cancer (including rectal adenocarcinoma), appendiceal cancer, small-bowel cancer, esophageal cancer, hepatobiliary cancer (including liver cancer, bile duct cancer and bile duct carcinoma), bladder cancer, ovarian cancer, duodenal papillary cancer and a solid tumor, particularly when the cancer or tumor harbors a KRAS G12C mutation.

In embodiments of the invention, the cancer to be treated by Compound A, or by the combinations or in the methods of the invention, is selected from the group consisting of melanoma, gastric lymphoma, Burkitt's lymphoma, head & neck squamous cell carcinoma, oral cancer, pancreatic adenocarcinoma, non-small cell lung carcinoma, esophageal squamous cell carcinoma, gastric cancer, colorectal cancer, epithelial ovarian carcinoma and prostate cancer; optionally wherein the cancer harbors a KRAS G12C mutation and/or a CDKN2A mutation; or wherein the cancer harbors KRAS G12C, PIK3CA and CDKN2A mutations.

In embodiments of the invention, the cancer to be treated by Compound A, or by the combinations or in the methods of the invention, is selected from the group consisting of breast cancer, ovarian cancer, lung cancer, stomach cancer, gastric cancer and brain cancer; optionally wherein the cancer harbors a KRAS G12C mutation and/or a PIK3CA mutation; or wherein the cancer harbors KRAS G12C, PIK3CA and CDKN2A mutations.

The cancer may be at an early, intermediate, late stage or may be metastatic cancer. In some embodiments, the cancer is an advanced cancer. In some embodiments, the cancer is a metastatic cancer. In some embodiments, the cancer is a relapsed cancer. In some embodiments, the cancer is a refractory cancer. In some embodiments, the cancer is a recurrent cancer. In some embodiments, the cancer is an unresectable cancer.

The cancer may be at an early, intermediate, late stage or metastatic cancer.

Compound A and combinations of the invention may also be useful in the treatment of solid malignancies characterized by mutations of RAS.

Compound A and combinations of the invention may also be useful in the treatment of solid malignancies characterized by one or more mutations of KRAS, in particular G12C mutations in KRAS.

The present invention provides Compound A and combinations of the invention for use in the treatment of a cancer or solid tumor characterized by an acquired KRAS alteration which is selected from G12D/R/V/W, G13D, Q61H, R68S, H95D/Q/R, Y96C, Y96 D and high-level amplification of the KRASG12C allele, or characterized by an acquired bypass mechanisms of resistance, These bypass mechanisms of resistance include MET amplification; activating mutations in NRAS, BRAF, MAP2K1, and RET; oncogenic fusions involving ALK, RET, BRAF, RAF1, and FGFR3; and loss-of-function mutations in NF1 and PTEN.

Thus, as a further embodiment, the present invention provides a combination of the invention for use in therapy. The present invention also provides a triple combination consisting of Compound A, or a pharmaceutically acceptable salt thereof, a SHP2 inhibitor such as TNO155, or a pharmaceutically acceptable salt thereof, and third therapeutically active agent. As a further embodiment, the present invention provides a combination of the invention for use in therapy. In a preferred embodiment, the therapy or the therapy which the medicament is useful for is selected from a disease which may be treated by inhibition of RAS mutant proteins, in particular, KRAS, HRAS or NRAS G12C mutant proteins. In another embodiment, the invention provides a method of treating a disease, which is treated by inhibition of a RAS mutant protein, in particular, a G12C mutant of either KRAS, HRAS or NRAS protein, in a subject in need thereof, wherein the method comprises the administration of a therapeutically effective amount of a combination of the invention, to the subject.

In a more preferred embodiment, the disease is selected from the afore-mentioned list, suitably non-small cell lung cancer, colorectal cancer and pancreatic cancer.

In a preferred embodiment, the therapy is for a disease, which may be treated by inhibition of a RAS mutant protein, in particular, a G12C mutant of either KRAS, HRAS or NRAS protein. In a more preferred embodiment, the disease is selected from the afore-mentioned list, suitably non-small cell lung cancer, colorectal cancer and pancreatic cancer, which is characterized by a G12C mutation in either KRAS, HRAS or NRAS.

In another embodiment is method of treating (e.g., one or more of reducing, inhibiting, or delaying progression) a cancer or a tumor in a subject comprising administering to a subject in need thereof a pharmaceutical composition comprising Compound A, or pharmaceutically acceptable salt thereof, in combination with a second therapeutic agent as described herein, optionally with a third combination.

The present invention therefore provides a method of treating (e.g., one or more of reducing, inhibiting, or delaying progression) cancer or tumor in a patient in need thereof, wherein the method comprises administering to the patient in need thereof, a therapeutically active amount of the combination of the invention, wherein the cancer is lung cancer (including lung adenocarcinoma and non-small cell lung cancer), colorectal cancer (including colorectal adenocarcinoma), pancreatic cancer (including pancreatic adenocarcinoma), uterine cancer (including uterine endometrial cancer), rectal cancer (including rectal adenocarcinoma) and a solid tumor, optionally wherein the cancer is KRAS-, NRAS- or HRAS-G12C mutant.

### Cancer or Tumor Refractory to KRAS G12C Inhibitors

The methods and combinations of the invention may be particularly useful for treating a cancer or tumor which is refractory or resistant to prior treatment with a KRAS G12C inhibitor. Examples of such a KRAS G12C inhibitor include Compound A, sotorasib (Amgen), adagrasib (Mirati), D-1553 (InventisBio), BI1701963 (Boehringer), GDC6036 (Roche), JNJ74699157 (J&J), X-Chem KRAS (X-Chem), LY3537982 (Lilly), BI1823911 (Boehringer), AS KRAS G12C (Ascentage Pharma), SF KRAS G12C (Sanofi), RMC032 (Revolution Medicine), JAB-21822 (Jacobio Pharmaceuticals), AST-KRAS G12C (Allist Pharmaceuticals), AZ KRAS G12C (Astra Zeneca), NYU-12VC1 (New York University), and RMC6291 (Revolution Medicines), or a pharmaceutically acceptable salt thereof. In one embodiment, the cancer. e.g. NSCLC, has previously been treated with a KRAS G12C inhibitor (e.g. sotorasib, adagrasib, D-1553, and GDC6036).

It is expected that a combination therapy which involves a KRAS G12 C inhibitor (e.g. Compound A, or a pharmaceutically active salt thereof, and second therapeutically active agent, optionally a third therapeutic agent would be particularly useful in overcoming this resistance.

The methods and combinations of the invention may be useful as first line therapy (or as second or more advanced lines of therapy). For example, the patient may be a treatment agnostic patient or a patient who has progressed and/or relapsed on previous therapy.

For example, the patient or subject to be treated by the methods and combinations of the invention include a patient suffering from cancer, e.g. KRAS G12C mutant NSCLC (including advanced (metastatic or unresectable) KRAS G12C mutant NSCLC), optionally wherein the patient has received and progressed on previous therapy.

In embodiments of the invention, the subject or patient to be treated and likely to benefit from treatment with Compound A monotherapy or combination therapy with a combination therapy as described herein is selected from:
a patient suffering from a KRAS G12C mutant solid tumor (e.g. advanced (metastatic or unresectable) KRAS G12C mutant solid tumor), optionally wherein the patient has received and failed standard of care therapy or is intolerant or ineligible to previous investigative and/or approved therapies;
a patient suffering from KRAS G12C mutant NSCLC (e.g., advanced (metastatic or unresectable) KRAS G12C mutant NSCLC), optionally wherein the patient who has received and failed a platinum-based chemotherapy regimen and an immune checkpoint inhibitor therapy either in combination or in sequence;
a patient suffering from KRAS G12C mutant CRC (e.g., advanced (metastatic or unresectable) KRAS G12C mutant CRC), optionally wherein the patient has received and failed standard of care therapy, including a fluoropyrimidine-, oxaliplatin-, and/or irinotecan-based chemotherapy; and
a patient suffering from KRAS G12C mutant NSCLC (e.g., advanced (metastatic or unresectable) KRAS G12C mutant NSCLC), optionally wherein the patient who has previously been treated with a KRAS G12C inhibitor (e.g. sotorasib, adagrasib, GDC6036 or D-1553).

Compound A alone or in combination with another therapeutic agent as described herein may be useful in the treatment of a patient which is selected from:
a patient with NSCLC whose tumors harbor the KRAS G12C tumor mutation and who has received a prior platinum based chemotherapy regimen and immune checkpoint inhibitor therapy either in combination or in sequence (G12Ci naive);
a patient with NSCLC whose tumors harbor the KRAS G12C tumor mutation and who has received a prior platinum based chemotherapy regimen and immune checkpoint inhibitor therapy either in combination or in sequence directly followed by one treatment line of a KRAS G12C inhibitor other than Compound A, e.g. sotorasib or adagrasib, given as a single agent and discontinued within 6 months of the first day of study treatment in this trial (G12Ci treated);
a patient with CRC whose tumors harbor the KRAS G12C tumor mutation and who has received fluoropyrimidine-, oxaliplatin-, or irinotecan-based chemotherapy.

In a further embodiment, the Compound A, or pharmaceutically acceptable salt thereof, administered to the subject in need thereof in an amount which is effective to treat the cancer.

In embodiments of the invention, the amounts of Compound A, or pharmaceutically acceptable salt thereof and the second therapeutic agent—and the third therapeutic agent, if present, are administered to the subject in need thereof and are effective in amounts which are effective to treat the cancer.

### Dosages and Dosing Regimens

When Compound A is used as monotherapy, the total daily recommended dose of Compound A is 400 mg, given once daily or twice daily, given continuously (i.e. with no drug holiday). The recommended dose for Compound A monotherapy is 100 mg BID given continuously, based on the observed safety, PK and efficacy data.

When Compound A is used as monotherapy or as combination therapy, it is preferably taken with food, e.g. immediately (within 30 minutes) following a meal.

Doses of the KRAS G12 C inhibitor and the second therapeutically active agent, and the third therapeutically active agent in the combination therapy according to the present invention are designed to be pharmacologically active and result in an anti-tumor response.

When the KRAS G12 C inhibitor is Compound A in a combination of the present invention, Compound A, or a pharmaceutically acceptable salt thereof, is administered at a therapeutically effective dose ranging from 50 to 1600 mg per day, e.g. from 200 to 1600 mg per day, or from 400 to 1600 mg or from 50 to 400 mg per day. The total daily dose of Compound A may be selected from 50, 100, 150, 200, 250, 300, 350, 400, 450, 500, 600, 800, 1000, 1200 and 1600 mg. For example, the total daily dose of Compound A may be selected from 100, 200, 300, 400, 600, 800, 1000, 1200 and 1600 mg.

The total daily dose of Compound A may be administered continuously, on a QD (once a day) or BID (twice a day) regimen. For example, Compound A may be administered at a dose of 200 mg BID (total daily dose of 400 mg), 400 mg QD (total daily dose 400 mg). Compound A may also be administered at a dose of 100 mg BID (total daily dose of 200 mg) or at a dose of 200 mg QD (total daily dose 200 mg). PK/PD modeling predicts sustained, high-level target occupancy at the recommended dose of 200 mg BID. 100 mg BID of Compound A is also predicted to allow for an adequate therapeutic window when combined with selected therapies.

When a SHP2 inhibitor is present and TNO155 the SHP2 inhibitor, in a combination of the present invention, doses of TNO 155 in the combinations of the present invention are designed to be pharmacologically active and have a potential for a synergistic anti-tumor effect while at the same time minimizing the possibility of unacceptable toxicity due to suppressive activities by both agents on MAPK pathway signaling. Thus TNO155 may be administered at a total daily dose ranging from 10 to 80 mg, or from 10 to 60 mg. For example, the total daily dose of TNO155 may be selected from 10, 15, 20, 30, 40, 60 and 80 mg. The total daily dose of TNO155 may be administered continuously, QD (once a day) or BID (twice a day) on QD or BID on a 2 weeks on/1 week off schedule. The total daily dose of TNO155 may be administered continuously, QD (once a day) or BID (twice a day) on QD or BID on continuously (i.e. without a drug holiday).

In combinations of the invention, Compound A is administered at a dose ranging from 50 to 1600 mg per day (e.g., 50, 100, 150, 200, 250, 300, 350, 400, 450, 500, 600, 800, 1000, 1200 or 1600 mg) or from 200 to 1600 mg per day (e.g., 200, 300, 400, 600, 800, 1000, 1200 or 1600 mg) and TNO155 is administered at a dose ranging from 10 to 80 mg per day (0, 15, 20, 30, 40, 60 or 80 mg), wherein Compound A is administered on a continuous schedule and TNO is administered either on a two week on/one week off schedule or on a continuous schedule.

In combinations of the invention, Compound A is administered on a continuous schedule at a dose ranging from 50 to 1600 mg per day (e.g., 50, 100, 150, 200, 250, 300, 350, 400, 450, 500, 600, 800, 1000, 1200 or 1600 mg) or from 200 to 1600 mg per day (e.g., 200, 300, 400, 600, 800, 1000, 1200 or 1600 mg), TNO155 is administered either on a two week on/one week off schedule or on a continuous schedule at a dose ranging from 10 to 80 mg (0, 15, 20, 30, 40, 60 or 80 mg).

An EGFR inhibitor such as cetuximab may be used in the combination therapy of the invention, in particular when the cancer to be treated is colorectal cancer. Cetuximab, when present, is used as a concentrated solution for infusion and administered intravenously (IV). Cetuximab may be administered weekly, with an initial dose of 400 mg/m² IV (typically administered as a 120-minute intravenous infusion), and subsequent doses of 250 mg/m²/week (administered as a 60-minute infusion every week). Alternatively, cetuximab may be administered biweekly, at initial and subsequent doses of 500 mg/m² once every two weeks. Typically, the total daily dose of Compound A in the combinations of the invention may be selected from 100 mg to 400 mg, e.g. from 200 mg to 400 mg. The total daily dose may be administered once daily or twice daily (BID) continuously.

Examples of dosing regimens for the combination of Compound A and cetuximab are Compound A QD or BID administered continuously in combination with cetuximab weekly dosing (initial dose 400 mg/m2 administered as a 120-minute intravenous infusion, subsequent doses 250 mg/m2 administered as a 60-minute infusion every week. Typically, the overall exposure of cetuximab may not exceed 500 mg/m2 every 2 weeks or 400 mg/m2 initial dose followed by 250 mg/m2 weekly.

Typical dose levels of Compound A in combination with cetuximab may be as follows:

| Dosing | | |
|---|---|---|
| schedules | Compound A | Cetuximab biweekly schedule |
| 1 | 100 mg once daily | 300, 400 or 500 mg/m² Q2W |
| 2 | 100 mg twice daily | 300, 400 or 500 mg/m² Q2W |
| 3 | 200 mg twice daily | 300, 400 or 500 mg/m² Q2W |

A MEK inhibitor such as trametinib may be used in the combination therapy of the invention. Trametinib may be administered continuously (i.e. with no drug holiday) at a dose of 0.5 mg, 1 mg or 2 mg once daily (QD). Based on clinical PK and PD data, the 1 mg QD dose of trametinib is considered potentially pharmacologically active. Compound A and/or trametinib may be administered with food. Typically, the total daily dose of Compound A in the combinations of the invention may be selected from 100 mg to 400 mg, e.g. from 200 mg to 400 mg. The total daily dose may be administered once daily or twice daily (BID) continuously.

Typical dose levels of Compound A in combination with trametinib may be as follows:

| Dosing | | |
|---|---|---|
| schedules | Compound A | Tametinib |
| 1 | 100 mg once daily | 0.5 mg once daily |
| 2 | 100 mg twice daily | 0.5 mg once daily |
| 3 | 100 mg twice daily | 1 mg once daily |
| 4 | 200 mg twice daily | 1 mg once daily |
| 5 | 200 mg twice daily | 2 mg once daily |

A CDK4/6 inhibitor such as palbociclib or ribociclib may be used in the combination therapy of the invention. When ribociclib is used as a combination partner, it may be administered at a total daily dose of 100 mg to 600 mg QD, 3 weeks off/1 week off. For example, ribociclib may be administered once daily at a dose of 100 mg, 200 mg, 300 mg, 400 mg or 600 mg. Typically, the total daily dose of Compound A in the combinations of the invention may be selected from 100 mg to 400 mg, e.g. from 200 mg to 400 mg. The total daily dose may be administered once daily or twice daily (BID) continuously.

Typical dose levels of Compound A in combination with ribociclib may be as follows:

| Dosing | | Ribociclib |
|---|---|---|
| schedules | Compound A | (3 weeks on, 1 week off) |
| 1 | 100 mg once daily | 200 mg once daily |
| 2 | 100 mg twice daily | 200 mg once daily |
| 3 | 100 mg twice daily | 200 mg once daily |
| 4 | 200 mg twice daily | 400 mg once daily |
| 5 | 200 mg twice daily | 600 mg once daily |

### Pharmaceutical Compositions

The KRAS G12 C inhibitor (e.g. Compound A, or a pharmaceutically acceptable salt thereof) may be administered either simultaneously with, or before or after, one or more (e.g., one or two) other therapeutically active agents. Compound A, or a pharmaceutically acceptable salt thereof, may be administered separately, by the same or different route of administration, or together in the same pharmaceutical composition as the other therapeutically active agents.

In another aspect, the present invention provides pharmaceutically acceptable compositions which comprise a therapeutically effective amount of one or more (e.g., one or two) therapeutic agents selected from a KRAS G12C inhibitor (e.g. Compound A), SHP2 inhibitor (such as TNO155) and optionally a third agent, as described herein, formulated together with one or more pharmaceutically acceptable carriers (additives) and/or diluents.

In another aspect, the present invention provides a pharmaceutical composition comprising one, two or three compounds present in the combination of the invention, or a pharmaceutically acceptable salt thereof, and a pharmaceutically acceptable carrier. In another aspect, the present invention provides a pharmaceutical composition comprising a KRAS G12C inhibitor, such as Compound A, or a pharmaceutically acceptable salt thereof, and one or more (e.g., one or two) therapeutically active agents selected from a SHP2 inhibitor such as TNO155, or a pharmaceutically acceptable salt thereof and a third therapeutically active agent. In a further embodiment, the composition comprises at least two pharmaceutically acceptable carriers, such as those described herein. Preferably, pharmaceutically acceptable carriers are sterile. The pharmaceutical composition can be formulated for particular routes of administration such as oral administration, parenteral administration, and rectal administration, etc. In addition, the pharmaceutical compositions of the present invention can be made up in a solid form (including without limitation capsules, tablets, pills, granules, powders or suppositories), or in a liquid form (including without limitation solutions, suspensions or emulsions). The pharmaceutical compositions can be subjected to conventional pharmaceutical operations such as sterilization and/or can contain conventional inert diluents, lubricating agents, or buffering agents, as well as adjuvants, such as preservatives, stabilizers, wetting agents, emulsifiers and buffers, etc.

Typically, the pharmaceutical compositions are tablets or gelatin capsules comprising the active ingredient together with one or more of:
a) diluents, e.g., lactose, dextrose, sucrose, mannitol, sorbitol, cellulose and/or glycine;
b) lubricants, e.g., silica, talcum, stearic acid, its magnesium or calcium salt and/or polyethyleneglycol;
c) binders, e.g., magnesium aluminum silicate, starch paste, gelatin, tragacanth, methylcellulose, sodium carboxymethylcellulose and/or polyvinylpyrrolidone;
d) disintegrants, e.g., starches, agar, alginic acid or its sodium salt, or effervescent mixtures; and
e) absorbents, colorants, flavors and sweeteners.

In an embodiment, the pharmaceutical compositions are capsules comprising the active ingredient only.

Tablets may be either film coated or enteric coated according to methods known in the art.

Suitable compositions for oral administration include an effective amount of a compound in a combination of the invention in the form of tablets, lozenges, aqueous or oily suspensions, dispersible powders or granules, emulsion, hard or soft capsules, or syrups or elixirs, solutions or solid dispersion. Compositions intended for oral use are prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable prepa-rations. Tablets may contain the active ingredient in admixture with nontoxic pharmaceutically acceptable excipients which are suitable for the manufacture of tablets. These excipients are, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets are uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Certain injectable compositions are aqueous isotonic solutions or suspensions, and suppositories are advantageously prepared from fatty emulsions or suspensions. Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1-75%, or contain about 1-50%, of the active ingredient.

Suitable compositions for transdermal application include an effective amount of a compound of the invention with a suitable carrier. Carriers suitable for transdermal delivery include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound of the skin of the host at a controlled and predetermined rate over a prolonged period of time, and means to secure the device to the skin.

Suitable compositions for topical application, e.g., to the skin and eyes, include aqueous solutions, suspensions, ointments, creams, gels or sprayable formulations, e.g., for delivery by aerosol or the like. Such topical delivery systems will in particular be appropriate for dermal application, e.g., for the treatment of skin cancer, e.g., for prophylactic use in sun creams, lotions, sprays and the like. They are thus particularly suited for use in topical, including cosmetic, for-mulations well-known in the art. Such may contain solubilizers, stabilizers, tonicity enhancing agents, buffers and preservatives.

As used herein, a topical application may also pertain to an inhalation or to an intranasal application. They may be conveniently delivered in the form of a dry powder (either alone, as a mixture, for example a dry blend with lactose, or a mixed component particle, for example with phospholipids) from a dry powder inhaler or an aerosol spray presentation from a pressurised container, pump, spray, atomizer or nebuliser, with or without the use of a suitable propellant.

In one embodiment, the invention provides a product comprising Compound A, or a pharmaceutically acceptable salt thereof, and at least one other therapeutic agent as a combined preparation for simultaneous, separate or sequential use in therapy. In one embodiment, the therapy is the treatment of a disease or condition characterized by a KRAS, HRAS or NRAS G12C mutation. Products provided as a combined preparation include a composition comprising the compound of the present invention and one or more (e.g., one or two) therapeutically active agents selected from a SHP2 inhibitor (such as TNO155, or a pharmaceutically acceptable salt thereof), a KRAS inhibitor (such as Compound A, or a pharmaceutically acceptable salt, thereof, and the other therapeutic agent(s) in separate form, e.g. in the form of a kit.

In one embodiment, the invention provides a pharmaceutical composition comprising a compound of the present invention and another therapeutic agent(s). Optionally, the pharmaceutical composition may comprise a pharmaceutically acceptable carrier, as described above.

In one embodiment, the invention provides a kit comprising two or more separate pharmaceutical compositions, at least one of which contains Compound A, or a pharmaceutically acceptable salt thereof; TN0155, or a pharmaceutically acceptable salt thereof, and third therapeutically active agent as described herein. In one embodiment, the kit comprises means for separately retaining said compositions, such as a container, divided bottle, or divided foil packet. An example of such a kit is a blister pack, as typically used for the packaging of tablets, capsules and the like.

The kit of the invention may be used for administering different dosage forms, for example, oral and parenteral, for administering the separate compositions at different dosage intervals, or for titrating the separate compositions against one another. To assist compliance, the kit of the invention typically comprises directions for administration.

In the combination therapies of the invention, the compound of the present invention and the other therapeutic agent may be manufactured and/or formulated by the same or different manufacturers. Moreover, the compound of the present invention and the other therapeutic may be brought together into a combination therapy: (i) prior to release of the combination product to physicians (e.g. in the case of a kit comprising the compound of the present invention and the other therapeutic agent); (ii) by the physician themselves (or under the guidance of the physician) shortly before administration; (iii) in the patient themselves, e.g. during sequential administration of the compound of the present invention and the other therapeutic agent. The compound of the present invention may be administered either simultaneously with, or before or after, one or more other therapeutic agent. The compound of the present invention may be administered separately, by the same or different route of administration, or together in the same pharmaceutical composition as the other agents.

In general, a suitable daily dose of the combination of the invention will be that amount of each compound which is the lowest dose effective to produce a therapeutic effect.

In another aspect, the present invention provides pharmaceutically acceptable compositions which comprise a therapeutically-effective amount of one or more of the subject compounds, as described above, formulated together with one or more pharmaceutically acceptable carriers (additives) and/or diluents.

### Definitions

The general terms used hereinbefore and hereinafter preferably have within the context of this disclosure the following meanings, unless otherwise indicated, where more general terms wherever used may, independently of each other, be replaced by more specific definitions or remain, thus defining more detailed embodiments of the invention:

In particular, where a dose or dosage is mentioned, it is intended to include a range around the specified value of plus or minus 10%, or plus or minus 5%.

As is customary in the art, dosages refer to the amount of the therapeutic agent in its free form. For example, when a dosage of 20 mg of TNO155 is referred to, and TNO155 is used as its succinate salt, the amount of the therapeutic agent used is equivalent to 20 mg of the free form of TNO155.

The term “subject” or “patient” as used herein is intended to include animals, which are capable of suffering from or afflicted with a cancer or any disorder involving, directly or indirectly, a cancer. Examples of subjects include mammals, e.g., humans, apes, monkeys, dogs, cows, horses, pigs, sheep, goats, cats, mice, rabbits, rats, and transgenic non-human animals. In an embodiment, the subject is a human, e.g., a human suffering from, at risk of suffering from, or potentially capable of suffering from cancers.

The term “treating” or “treatment” as used herein comprises a treatment relieving, reducing or alleviating at least one symptom in a subject or effecting a delay of progression of a disease. For example, treatment can be the diminishment of one or several symptoms of a disorder or partial or complete eradication of a disorder, such as cancer. Within the meaning of the present disclosure, the term “treat” also denotes to arrest, delay the onset (i.e., the period prior to clinical manifestation of a disease) and/or reduce the risk of developing or worsening a disease.

“Treatment” may also be determined by efficacy and/or pharmacodynamic endpoints and may be defined as an improvement in one or more of safety, efficacy and tolerability. Efficacy of the monotherapy or the combination therapy may be determined by determining Best Overall Response (BOR), Overall Response Rate (ORR), Duration of Response (DOR), Disease Control Rate (DCR), Progression Free Survival, (PFS) and Overall Survival (OS) per RECIST v.1.1.

“Best overall response” (BOR) rate is defined as the best response recorded from the start of the treatment until disease progression/recurrence and according to RECIST 1.1.

“Overall response rate” (ORR) is defined as the proportion of patients with a BOR of CR or PR according to RECIST 1.1.

“Duration of Response” (DOR) per RECIST 1.1 is the time between the first documented response (CR or PR) and the date of progression or death due to any cause. Here, death due to any cause is considered as an event to be conservative and align with PFS event definition.

“Disease control rate” (DCR) per RECIST 1.1 is defined as the proportion of patients with a BOR of CR, PR, or SD according to RECIST 1.1.

“Progression Free Survival” (PFS) per RECIST 1.1 is defined as the time from the date of start of treatment to the date of the first documented progression according to RECIST 1.1, or death due to any cause. If a patient has not had an event, PFS will be censored at the date of last adequate tumor assessment.

“Overall survival” (OS) is defined as the number of days between the date of start of study treatment to the date of death due to any cause. If no death is reported prior to study termination or analysis cut off, survival will be censored at the date of last known date patient alive prior to/on the cut off date. Survival time for patients with no post-baseline survival information will be censored at the date of start of treatment.

“Treatment” may also be defined as an improvement in a reduction of adverse effects of the monotherapy with Compound A, or the combination therapy as described herein.

The terms “comprising” and “including” are used herein in their open-ended and non-limiting sense unless otherwise noted.

The terms “a” and “an” and “the” and similar references in the context of describing the invention (especially in the context of the following claims) are to be construed to cover both the singular and the plural, unless otherwise indicated herein or clearly contradicted by context. Where the plural form is used for compounds, salts, and the like, this is taken to mean also a single compound, salt, or the like.

The term “combination therapy” or “in combination with” refers to the administration of two or more therapeutic agents to treat a condition or disorder described in the present disclosure (e.g., cancer). Such administration encompasses co-administration of these therapeutic agents in a substantially simultaneous manner, such as in a single capsule having a fixed ratio of active ingredients. Alternatively, such administration encompasses co-administration in multiple, or in separate containers (e.g., capsules, powders, and liquids) for each active ingredient. Powders and/or liquids may be reconstituted or diluted to a desired dose prior to administration. In addition, such administration also encompasses use of each type of therapeutic agent in a sequential manner, either at approximately the same time or at different times. In either case, the treatment regimen will provide beneficial effects of the drug combination in treating the conditions or disorders described herein.

The combination therapy can provide “synergy” and prove “synergistic”, i.e., the effect achieved when the active ingredients used together is greater than the sum of the effects that results from using the compounds separately. A synergistic effect can be attained when the active ingredients are: (1) co-formulated and administered or delivered simultaneously in a combined, unit dosage formulation; (2) delivered by alternation or in parallel as separate formulations; or (3) by some other regimen. When delivered in alternation therapy, a synergistic effect can be attained when the compounds are administered or delivered sequentially, e.g., by different injections in separate syringes. In general, during alternation therapy, an effective dosage of each active ingredient is administered sequentially, i.e., serially, whereas in combination therapy, effective dosages of two or more active ingredients are administered together. Synergistic effect, as used herein, refers to action of two therapeutic agents such as, for example, a compound TNO155 as a SHP2 inhibitor and Compound A, producing an effect, for example, slowing the symptomatic progression of a proliferative disease, particularly cancer, or symptoms thereof, which is greater than the simple addition of the effects of each drug administered by themselves. A synergistic effect can be calculated, for example, using suitable methods such as the Sigmoid-Emax equation (Holford, N. H. G. and Scheiner, L. B., Clin. Pharmacokinet. 6: 429-453 (1981)), the equation of Loewe additivity (Loewe, S. and Muischnek, H., Arch. Exp. Pathol Pharmacol. 114: 313-326 (1926)) and the median-effect equation (Chou, T. C. and Talalay, P., Adv. Enzyme Regul. 22: 27-55 (1984)). Each equation referred to above can be applied to experimental data to generate a corresponding graph to aid in assessing the effects of the drug combination. The corresponding graphs associated with the equations referred to above are the concentration-effect curve, isobologram curve and combination index curve, respectively.

The term “pharmaceutical combination” as used herein refers to either a fixed combination in one dosage unit form, or non-fixed combination or a kit of parts for the combined administration where two or more therapeutic agents may be administered independently at the same time or separately within time intervals, especially where these time intervals allow that the combination partners show a cooperative, e.g. synergistic effect.

The phrase “therapeutically-effective amount” as used herein means that amount of a compound, material, or composition comprising a compound of the present invention which is effective for producing some desired therapeutic effect in at least a sub-population of cells in an animal at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As set out above, certain embodiments of the present compounds may contain a basic functional group, such as amino or alkylamino, and are, thus, capable of forming pharmaceutically-acceptable salts with pharmaceutically-acceptable acids. The term “pharmaceutically-acceptable salts” in this respect, refers to the relatively non-toxic, inorganic and organic acid addition salts of compounds of the present invention. These salts can be prepared in situ in the administration vehicle or the dosage form manufacturing process, or by separately reacting a purified compound of the invention in its free base form with a suitable organic or inorganic acid, and isolating the salt thus formed during subsequent purification. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, for example, Berge et al. (1977) “Pharmaceutical Salts”, J Pharm. Sci. 66:1-19).

The pharmaceutically acceptable salts of the subject compounds include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like. The pharmaceutically acceptable salt of TNO155, for example, is succinate.

In the combination of the invention, Compound A, TNO155 and a third therapeutically active agent, is also intended to represent unlabeled forms as well as isotopically labeled forms of the compounds. Isotopically labeled compounds have one or more atoms replaced by an atom having a selected atomic mass or mass number. Examples of isotopes that can be incorporated into TNO155 and a third therapeutically active agent include isotopes, where possible, of hydrogen, carbon, nitrogen, oxygen, and chlorine, for example, ²H, ³H, ¹¹C, ¹³C, ¹⁴C, ¹⁵N, ³⁵S ³⁶Cl. The invention includes isotopically labeled TNO155 and a PD-1 inhibitor, for example into which radioactive isotopes, such as ³H and ¹⁴C, or non-radioactive isotopes, such as ²H and ¹³C, are present. Isotopically labelled TNO155 and a third therapeutically active agent are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. Isotopically-labeled compounds of the invention can generally be prepared by conventional techniques known to those skilled in the art or by processes analogous to those described in the accompanying Examples using appropriate isotopically-labeled reagents.

Further, substitution with heavier isotopes, particularly deuterium (i.e., ²H or D) may afford certain therapeutic advantages resulting from greater metabolic stability, for example increased in vivo half-life or reduced dosage requirements or an improvement in therapeutic index. It is understood that deuterium in this context is regarded as a substituent of either Compound A, TNO155 or a third therapeutically active agent inhibitor. The concentration of such a heavier isotope, specifically deuterium, may be defined by the isotopic enrichment factor. The term “isotopic enrichment factor” as used herein means the ratio between the isotopic abundance and the natural abundance of a specified isotope. If a substituent in the compounds of the present invention is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

In Compound A, a methyl group, e.g. on the indazolyl ring, may be deuterated or perdeuterated.

### EXAMPLES

### Example 1: Preparation of 1-{6-[(4M)-4-(5-Chloro-6-methyl-1H-indazol-4-yl)-5-methyl-3-(1-methyl-1H-indazol-5-yl)-1H-pyrazol-1-yl]-2-azaspiro[3.3]heptan-2-yl}prop-2-en-1-one (Compound A

A synthesis of 1-{6-[(4M)-4-(5-Chloro-6-methyl-1H-indazol-4-yl)-5-methyl-3-(1-methyl-1H-indazol-5-yl)-1H-pyrazol-1-yl]-2-azaspiro[3.3]heptan-2-yl}prop-2-en-1-one (Compound A) is as described below.

Compound A is also known by the name “a(R)-1-(6-(4-(5-chloro-6-methyl-1H-indazol-4-yl)-5-methyl-3-(1-methyl-1H-indazol-5-yl)-1H-pyrazol-1-yl)-2-azaspiro[3.3]heptan-2-yl)prop-2-en-1-one”.

### General Methods and Conditions:

Temperatures are given in degrees Celsius. If not mentioned otherwise, all evaporations are performed under reduced pressure, typically between about 15 mm Hg and 100 mm Hg (20-133 mbar).

Abbreviations used are those conventional in the art.

Mass spectra were acquired on LC-MS, SFC-MS, or GC-MS systems using electrospray, chemical and electron impact ionization methods with a range of instruments of the following configurations: Waters Acquity UPLC with Waters SQ detector or Mass spectra were acquired on LCMS systems using ESI method with a range of instruments of the following configurations: Waters Acquity LCMS with PDA detector. [M+H]⁺ refers to the protonated molecular ion of the chemical species.

NMR spectra were run with Bruker Ultrashield™400 (400 MHz), Bruker Ultrashield™600 (600 MHz) and Bruker Ascend™400 (400 MHz) spectrometers, both with and without tetramethylsilane as an internal standard. Chemical shifts (6-values) are reported in ppm downfield from tetramethylsilane, spectra splitting pattern are designated as singlet (s), doublet (d), triplet (t), quartet (q), multiplet, unresolved or more overlapping signals (m), broad signal (br). Solvents are given in parentheses. Only signals of protons that are observed and not overlapping with solvent peaks are reported.

Celite: Celite® (the Celite corporation)=filtering aid based on diatomaceous earth Phase separator: Biotage—Isolute phase separator—(Part number: 120-1908-F for 70 mL and part number: 120-1909-J for 150 mL)

SiliaMetS® Thiol: SiliCYCLE thiol metal scavenger—(R51030B, Particle Size: 40-63 μm).

### Instrumentation

Microwave: All microwave reactions were conducted in a Biotage Initiator, irradiating at 0-400 W from a magnetron at 2.45 GHz with Robot Eight/Robot Sixty processing capacity, unless otherwise stated.

UPLC-MS and MS analytical Methods: Using Waters Acquity UPLC with Waters SQ detector.

UPLC-MS-1: Acquity HSS T3; particle size: 1.8 μm; column size: 2.1×50 mm; eluent A: H₂O+0.05% HCOOH+3.75 mM ammonium acetate; eluent B: CH₃CN+0.04% HCOOH; gradient: 5 to 98% B in 1.40 min then 98% B for 0.40 min; flow rate: 1 mL/min; column temperature: 60° C.

UPLC-MS-3: Acquity BEH C18; particle size: 1.7 μm; column size: 2.1×50 mm; eluent A: H₂O+4.76% isopropanol+0.05% HCOOH+3.75 mM ammonium acetate; eluent B: isopropanol+0.05% HCOOH; gradient: 1 to 98% B in 1.7 min then 98% B for 0.1 min; flow rate: 0.6 mL/min; column temperature: 80° C.

UPLC-MS-4: Acquity BEH C18; particle size: 1.7 μm; column size: 2.1×100 mm; eluent A: H₂O+4.76% isopropanol+0.05% HCOOH+3.75 mM ammonium acetate; eluent B: isopropanol+0.05% HCOOH; gradient: 1 to 60% B in 8.4 min then 60 to 98% B in 1 min; flow rate: 0.4 mL/min; column temperature: 80° C.

UPLC-MS-6: Acquity BEH C18; particle size: 1.7 μm; column size: 2.1×50 mm; eluent A: H₂O+0.05% HCOOH+3.75 mM ammonium acetate; eluent B: isopropanol+0.05% HCOOH; gradient: 5 to 98% B in 1.7 min then 98% B for 0.1 min; flow rate: 0.6 mL/min; column temperature: 80° C.

### Preparative Methods:

Chiral SFC methods:

C-SFC-1: column: Amylose-C NEO 5 μm; 250×30 mm; mobile phase; flow rate: 80 mL/min; column temperature: 40° C.; back pressure: 120 bar.

C-SFC-3: column: Chiralpak AD-H 5 μm; 100×4.6 mm; mobile phase; flow rate: 3 mL/min; column temperature: 40° C.; back pressure: 1800 psi.

### Abbreviations

| Abbreviation | Description |
|---|---|
| AcCN, ACN | acetonitrile |
| Ac₂O | acetic anhydride |
| AcOH | acetic acid |
| AIBN | 2,2′-azobis(2-methylpropionitrile) |
| aq. | aqueous |
| Ar | argon |
| B₂Pin₂ | 4,4,4′,4′,5,5,5′,5′-Octamethyl-2,2′-bi(1,3,2-dioxaborolane) |
| BPR | back pressure |
| brine | saturated aqueous sodium chloride |
| n-BuLi | n-butyl lithium |
| conc. | concentrated |
| DAST | N,N-diethyl-1,1,1-trifluoro-λ⁴-sulfanamine |
| DCE | dichloroethane |
| DCM | dichloromethane |
| DEA | diethylamine |
| DHP | 3,4-dihydropyran |
| DIPEA | N,N-diisopropylethylamine, N-ethyl-N-isopropylpropan-2-amine |
| DMA | N,N-dimethylacetamide |
| DMAP | N,N-dimethylpyridin-4-amine |
| DMF | N,N-Dimethylformamide |
| DMSO | dimethylsulfoxide |
| DMSO-d₆ | hexadeuterodimethyl sulfoxide |
| dppf | 1,1′-bis(diphenylphosphanyl) ferrocene |
| ee | enantiomeric excess |
| ESI | electrospray ionization |
| ESI-MS | electrospray ionization mass spectroscopy |
| EtOAc | ethyl acetate |
| g | gram |
| ug / μg | microgram |
| GBq | gigabecquerel |
| h | Hour (s) |
| HPLC | high-performance liquid chromatography |
| IPA | 2-propanol |
| KOAc | potassium acetate |
| L / mL / μL | litre / millilitre / microlitre |
| LC-MS or LCMS | liquid chromatography and mass spectroscopy |
| M | molar |
| uM / μM | micromolar |
| MeOH | methanol |
| min | minutes |
| MTBE | methyl tert-butyl ether |
| MS | mass spectroscopy |
| MW, mw | microwave |
| m/z | mass to charge ratio |
| N | normality |
| N₂ | nitrogen |
| NaOtBu | Sodium tert-butoxide |
| NBS | N-bromosuccinimide |
| NCS | N-chlorosuccinimide |
| NIS | N-iodosuccinimide |
| NEt₃, Et₃N, TEA | triethylamine |
| PDA | Photodiode array detector |
| NMR | nuclear magnetic resonance |
| Pd(PPh₃)₄ | tetrakis(triphenylphosphane)palladium(0) |
| iPrMgCl | Isopropylmagnesium chloride |
| PTSA | p-toluenesulfonic acid |
| RM | reaction mixture |
| RP | reversed phase |
| Rt | retention time |
| RT | room temperature |
| RuPhos | 2-dicyclohexylphosphino-2′,6′-diisopropoxybiphenyl |
| RuPhos-Pd-G3 | (2-dicyclohexylphosphino-2′,6′-diisopropoxy-1,1′-biphenyl)[2-(2′- |
| | amino-1,1′-biphenyl)]palladium(II) methanesulfonate |
| Sat. | saturated |
| SFC | supercritical fluid chromatography |
| SQ | Single-quadrupole |
| TBAF | Tetrabutylammonium fluoride |
| tBME, TBME, | tert-butyl methyl ether |
| TBMe | |
| TBq | terabecquerel |
| t-BuOH | tert-butanol |
| tBuXPhos-Pd-G3 | tBuXPhos-Pd-G3, [(2-Di-tert-butylphosphino-2′,4′,6′-triisopropyl- |
| | 1,1′-biphenyl)-2-(2′-amino-1,1′-biphenyl)] palladium(II) |
| | methanesulfonate |
| TFA | trifluoroacetic acid |
| THF | tetrahydrofuran |
| TLC | thin-layer chromatography |
| T₃P | propylphosphonic anhydride |
| TsCl | tosyl chloride, 4-Methylbenzene-1-sulfonyl chloride |
| UPLC | ultra-performance liquid chromatography |
| XPhos | 2-dicyclohexylphosphino-2′,4′,6′-triisopropylbiphenyl |
| XPhos-Pd-G3 | (2-dicyclohexylphosphino-2′,4′,6′-triisopropyl-1,1′-biphenyl)[2-(2′- |
| | amino-1,1′-biphenyl)]palladium(II) methanesulfonate |

All starting materials, building blocks, reagents, acids, bases, dehydrating agents, solvents, and catalysts utilized to prepare the compounds of the present invention are either commercially available or can be produced by organic synthesis methods known to one of ordinary skill in the art. Furthermore, the compounds of the present invention can be produced by organic synthesis methods known to one of ordinary skill in the art as shown in the following examples.

The structures of all final products, intermediates and starting materials are confirmed by standard analytical spectroscopic characteristics, e.g., MS, IR, NMR. The absolute stereochemistry of representative examples of the preferred (most active) atropisomers has been determined by analyses of X-ray crystal structures of complexes in which the respective compounds are bound to the KRAS G12C mutant. In all other cases where X-ray structures are not available, the stereochemistry has been assigned by analogy, assuming that, for each pair, the atropisomer exhibiting the highest activity in the covalent competition assay has the same configuration as observed by X-ray crystallography for the representative examples mentioned above. The absolute stereochemistry is assigned according to the Cahn-Ingold-Prelog rule.

### Synthesis of Intermediate C1: tert-butyl 6-(3-bromo-4-(5-chloro-6-methyl-1-(tetrahydro-2H-pyran-2-yl)-1H-indazol-4-yl)-5-methyl-1H-pyrazol-1-yl)-2-azaspiro[3.3]heptane-2-carboxylate

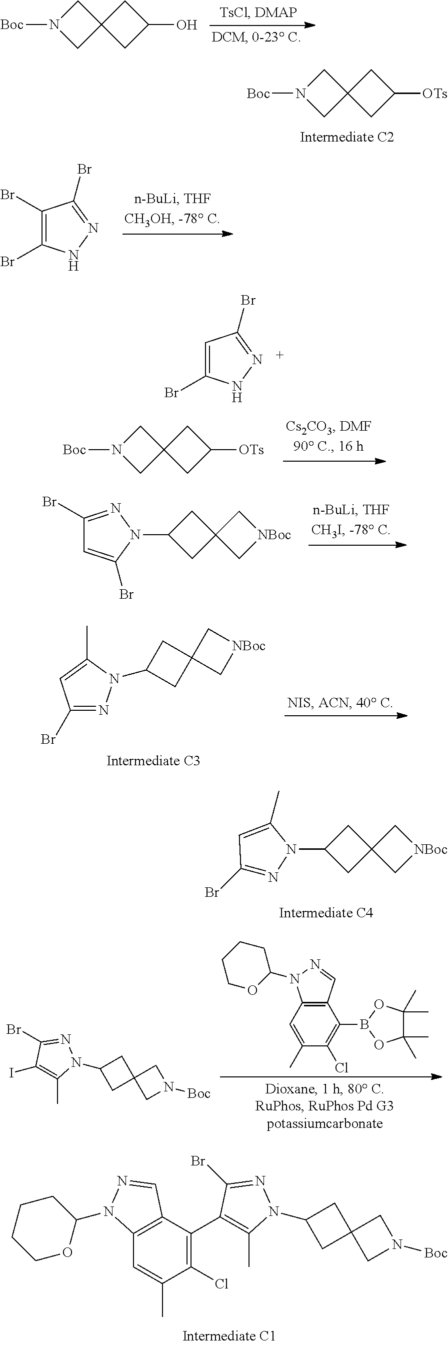

### Step C.1: tert-butyl 6-(tosyloxy)-2-azaspiro[3.3]heptane-2-carboxylate (Intermediate C2

To a solution of tert-butyl 6-hydroxy-2-azaspiro[3.3]heptane-2-carboxylate [1147557-97-8] (2.92 kg, 12.94 mmol) in DCM (16.5 L) were added DMAP (316.12 g, 2.59 mol) and TsCl (2.96 kg, 15.52 mol) at 20° C.-25° C. To the reaction mixture was added dropwise Et₃N (2.62 kg, 25.88 mol) at 10° C.-20° C. The reaction mixture was stirred 0.5 h at 5° C.-15° C. and then was stirred 1.5 h at 18° C.-28° C. After completion of the reaction, the reaction mixture was concentrated under vacuum. To the residue was added NaCl (5% in water, 23 L) followed by extraction with EtOAc (23 L). The combined aqueous layers were extracted with EtOAc (10 L×2). The combined organic layers were washed with NaHCO₃ (3% in water, 10 L×2)) and concentrated under vacuum to give the title compound. ¹H NMR (400 MHz, DMSO-d₆) δ 7.81-7.70 (m, 2H), 7.53-7.36 (m, 2H), 4.79-4.62 (m, 1H), 3.84-3.68 (m, 4H), 2.46-2.38 (m, 5H), 2.26-2.16 (m, 2H), 1.33 (s, 9H). UPLC-MS-1: Rt=1.18 min; MS m/z [M+H]⁺; 368.2.

### Step C.2: 3,5-dibromo-1H-pyrazole

To a solution of 3,4,5-tribromo-1H-pyrazole [17635-44-8] (55.0 g, 182.2 mmol) in anhydrous THF (550 mL) was added at −78° C. n-BuLi (145.8 mL, 364.5 mmol) dropwise over 20 min maintaining the internal temperature at −78° C./−60° C. The RM was stirred at this temperature for 45 min. Then the reaction mixture was carefully quenched with MeOH (109 mL) at −78° C. and stirred at this temperature for 30 min. The mixture was allowed to reach to 0° C. and stirred for 1 h. Then, the mixture was diluted with EtOAc (750 mL) and HCl (0.5 N, 300 mL) was added. The layers were concentrated under vacuum. The crude residue was dissolved in DCM (100 mL), cooled to −50° C. and petroleum ether (400 mL) was added. The precipitated solid was filtered and washed with n-hexane (250 mL×2) and dried under vacuum to give the title compound. ¹H NMR (400 MHz, DMSO-d₆) δ 13.5 (br s, 1H), 6.58 (s, 1H).

### Step C.3: tert-butyl 6-(35-dibromo-1H-pyrazol-1-yl)-2-azaspiro[3.3]heptane-2-carboxylate

To a solution of tert-butyl 6-(tosyloxy)-2-azaspiro[3.3]heptane-2-carboxylate (Intermediate C2) (Step C.1, 900 g, 2.40 mol) in DMF (10.8 L) was added Cs₂CO₃ (1988 g, 6.10 mol) and 3,5-dibromo-1H-pyrazole (Step C.2, 606 g, 2.68 mol) at 15° C. The reaction mixture was stirred at 90° C. for 16 h. The reaction mixture was poured into ice-water/brine (80 L) and extracted with EtOAc (20 L). The aqueous layer was re-extracted with EtOAc (10 L×2). The combined organic layers were washed with brine (10 L), dried (Na₂SO₄), filtered, and concentrated under vacuum. The residue was triturated with dioxane (1.8 L) and dissolved at 60° C. To the light yellow solution was slowly added water (2.2 L), and recrystallization started after addition of 900 mL of water. The resulting suspension was cooled down to 0° C., filtered, and washed with cold water. The filtered cake was triturated with n-heptane, filtered, then dried under vacuum at 40° C. to give the title compound. ¹H NMR (400 MHz, DMSO-d₆) δ 6.66 (s, 1H), 4.86-4.82 (m, 1H), 3.96-3.85 (m, 4H), 2.69-2.62 (m, 4H), 1.37 (s, 9H); UPLC-MS-3: Rt=1.19 min; MS m/z [M+H]⁺; 420.0/422.0/424.0.

### Step C.4: tert-butyl 6-(3-bromo-5-methyl-1H-pyrazol-1-yl)-2-azaspiro[3.3]heptane-2-carboxylate (Intermediate C3

To a solution of tert-butyl 6-(3,5-dibromo-1H-pyrazol-1-yl)-2-azaspiro[3.3]heptane-2-carboxylate (Step C.3, 960 g, 2.3 mol) in THF (9.6 L) was added n-BuLi (1.2 L, 2.5 mol) dropwise at −80° C. under an inert atmosphere. The reaction mixture was stirred 10 min at −80° C. To the reaction mixture was then added dropwise iodomethane (1633 g, 11.5 mol) at −80° C. After stirring for 5 min at −80° C., the reaction mixture was allowed to warm up to 18° C. The reaction mixture was poured into sat. aq. NH₄Cl solution (4 L) and extracted with DCM (10 L). The separated aqueous layer was re-extracted with DCM (5 L) and the combined organic layers were concentrated under vacuum. The crude product was dissolved in 1,4-dioxane (4.8 L) at 60° C., then water (8.00 L) was added dropwise slowly. The resulting suspension was cooled to 17° C. and stirred for 30 min. The solid was filtered, washed with water, and dried under vacuum to give the title compound. ¹H NMR (400 MHz, DMSO-d₆) δ 6.14 (s, 1H), 4.74-4.66 (m, 1H), 3.95-3.84 (m, 4H), 2.61-2.58 (m, 4H), 2.20 (s, 3H), 1.37 (s, 9H); UPLC-MS-1: Rt=1.18 min; MS m/z [M+H]⁺; 356.1/358.1.

### Step C.5: tert-butyl 6-(3-bromo-4-iodo-5-methyl-1H-pyrazol-1-yl)-2-azaspiro[3.3]heptane-2-carboxylate (Intermediate C4

To a solution of tert-butyl 6-(3-bromo-5-methyl-1H-pyrazol-1-yl)-2-azaspiro[3.3]heptane-2-carboxylate (Intermediate C3) (Step C.4, 350 g, 0.980 mol) in acetonitrile (3.5 L) was added NIS (332 g, 1.47 mol) at 15° C. The reaction mixture was stirred at 40° C. for 6 h. After completion of the reaction, the reaction mixture was diluted with EtOAc (3 L) and washed with water (5 L×2). The organic layer was washed with Na₂SO₃ (10% in water, 2 L), with brine (2 L), was dried (Na₂SO₄), filtered, and concentrated under vacuum to give the title compound. ¹H NMR (400 MHz, DMSO-d₆) δ 4.81-4.77 (m, 1H), 3.94-3.83 (m, 4H), 2.61-5.59 (m, 4H), 2.26 (s, 3H), 1.37 (s, 9H); UPLC-MS-1: Rt=1.31 min; MS m/z [M+H]⁺; 482.0/484.0.

### Step C.6: tert-butyl 6-(3-bromo-4-(5-chloro-6-methyl-1-(tetrahydro-2H-pyran-2-yl)-1H-indazol-4-yl)-5-methyl-1H-pyrazol-1-yl)-2-azaspiro[3.3]heptane-2-carboxylate (Intermediate C1

To a stirred suspension of tert-butyl 6-(3-bromo-4-iodo-5-methyl-1H-pyrazol-1-yl)-2-azaspiro[3.3]heptane-2-carboxylate (Intermediate C4) (Step C.5, 136 g, 282 mmol) and 5-chloro-6-methyl-1-(tetrahydro-2H-pyran-2-yl)-4-(4,4,5,5-tetramethyl-1,3,2-dioxaborolan-2-yl)-1H-indazole (Intermediate D1, 116 g, 310 mmol) in 1,4-dioxane (680 mL) was added aqueous K₃PO₄ (2M, 467 mL, 934 mmol) followed by RuPhos (13.1 g, 28.2 mmol) and RuPhos-Pd-G3 (14.1 g, 16.9 mmol). The reaction mixture was stirred at 80° C. for 1 h under inert atmosphere. After completion of the reaction, the reaction mixture was poured into 1M aqueous NaHCO₃ solution (1 L) and extracted with EtOAc (1 L×3). The combined organic layers were washed with brine (1 L×3), dried (Na₂SO₄), filtered, and concentrated under vacuum. The crude residue was purified by normal phase chromatography (eluent: Petroleum ether/EtOAc from I/O to 0/1) to give a yellow oil. The oil was dissolved in petroleum ether (1 L) and MTBE (500 mL), then concentrated in vacuo to give the title compound. ¹H NMR (400 MHz, DMSO-d₆) δ 7.81 (s, 1H), 7.66 (s, 1H), 5.94-5.81 (m, 1H), 4.90-4.78 (m, 1H), 3.99 (br s, 2H), 3.93-3.84 (m, 3H), 3.81-3.70 (m, 1H), 2.81-2.64 (m, 4H), 2.52 (s, 3H), 2.46-2.31 (m, 1H), 2.11-1.92 (m, 5H), 1.82-1.67 (m, 1H), 1.64-1.52 (m, 2H), 1.38 (s, 9H); UPLC-MS-3: Rt=1.30 min; MS m/z [M+H]⁺; 604.1/606.1.

### Synthesis of Intermediate D1: 5-chloro-6-methyl-1-(tetrahydro-2H-pyran-2-yl)-4-(4,4,5,5-tetramethyl-1,3,2-dioxaborolan-2-yl)-1H-indazole

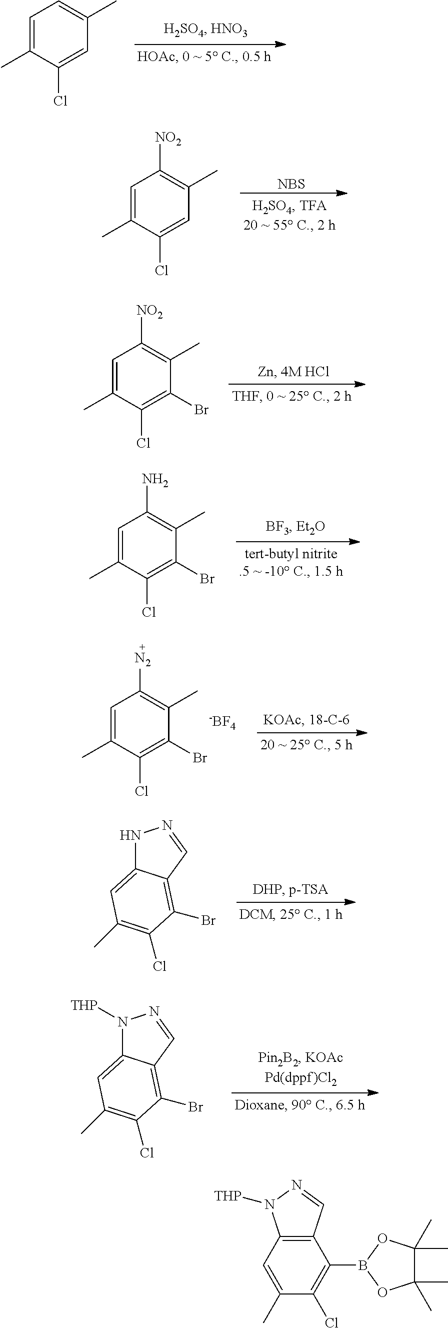

### Step D.1: 1-chloro-2,5-dimethyl-4-nitrobenzene

To an ice-cooled solution of 2-chloro-1,4-dimethylbenzene (3.40 kg, 24.2 mol) in AcOH (20.0 L) was added H₂SO₄ (4.74 kg, 48.4. mol, 2.58 L) followed by a dropwise addition (dropping funnel) of a cold solution of HNO₃ (3.41 kg, 36.3 mol, 2.44 L, 67.0% purity) in H₂SO₄ (19.0 kg, 193. mol, 10.3 L). The reaction mixture was then allowed to stir at 0-5° C. for 0.5 h. The reaction mixture was poured slowly into crushed ice (35.0 L) and the yellow solid precipitated out. The suspension was filtered and the cake was washed with water (5.00 L×5) to give a yellow solid which was suspended in MTBE (2.00 L) for 1 h, filtered, and dried to give the title compound as a yellow solid. ¹H NMR (400 MHz, CDCl₃) δ 7.90 (s, 1H), 7.34 (s, 1H), 2.57 (s, 3H), 2.42 (s, 3H).

### Step D.2: 3-bromo-2-chloro-1,4-dimethyl-5-nitrobenzene

To a cooled solution of 1-chloro-2,5-dimethyl-4-nitrobenzene (Step D.1, 2.00 kg, 10.8 mol) in TFA (10.5 L) was slowly added concentrated H₂SO₄ (4.23 kg, 43.1 mol, 2.30 L) and the reaction mixture was stirred at 20° C. NBS (1.92 kg, 10.8 mol) was added in small portions and the reaction mixture was heated at 55° C. for 2 h. The reaction mixture was cooled to 25° C., then poured into crushed ice solution to obtain a pale white precipitate which was filtered through vacuum, washed with cold water and dried under vacuum to give the title compound as a yellow solid which was used without further purification in the next step. ¹H NMR (400 MHz, CDCl₃) δ 7.65 (s, 1H), 2.60 (s, 3H), 2.49 (s, 3H).

### Step D.3: 3-bromo-4-chloro-2,5-dimethylaniline

To an ice-cooled solution of 3-bromo-2-chloro-1,4-dimethyl-5-nitrobenzene (Step D.2, 2.75 kg, 10.4 mol) in THF (27.5 L) was added HCl (4M, 15.6 L) then Zn (2.72 kg, 41.6 mol) in small portions. The reaction mixture was allowed to stir at 25° C. for 2 h. The reaction mixture was basified by addition of a sat. aq. NaHCO₃ solution (until pH=8). The mixture was diluted with EtOAc (2.50 L) and stirred vigorously for 10 min and then filtered through a pad of celite. The organic layer was separated and the aqueous layer was re-extracted with EtOAc (3.00 L×4). The combined organic layers were washed with brine (10.0 L), dried (Na₂SO₄), filtered and concentrated under vacuum to give the title compound as a yellow solid which was used without further purification in the next step. ¹H NMR (400 MHz, DMSO-d₆) δ 6.59 (s, 1H), 5.23 (s, 2H), 2.22 (s, 3H), 2.18 (s, 3H).

### Step D.4: 3-bromo-4-chloro-2,5-dimethylbenzenediazonium tetrafluoroborate

BF₃·Et₂O (2.00 kg, 14.1 mol, 1.74 L) was dissolved in DCM (20.0 L) and cooled to −5 to −10° C. under nitrogen atmosphere. A solution of 3-bromo-4-chloro-2,5-dimethylaniline (Step D.3, 2.20 kg, 9.38 mol) in DCM (5.00 L) was added to above reaction mixture and stirred for 0.5 h. Tert-butyl nitrite (1.16 kg, 11.3 mol, 1.34 L) was added dropwise and the reaction mixture was stirred at the same temperature for 1.5 h. TLC (petroleum ether:EtOAc=5:1) showed that starting material (R_{f}=0.45) was consumed completely. MTBE (3.00 L) was added to the reaction mixture to give a yellow precipitate, which was filtered through vacuum and washed with cold MTBE (1.50 L×2) to give the title compound as a yellow solid which was used without further purification in the next step.

### Step D.5: 4-bromo-5-chloro-6-methyl-1H-indazole

To 18-Crown-6 ether (744 g, 2.82 mol) in chloroform (20.0 L) was added KOAc (1.29 kg, 13.2 mol) and the reaction mixture was cooled to 20° C. Then 3-bromo-4-chloro-2,5-dimethylbenzenediazonium tetrafluoroborate (Step D.4, 3.13 kg, 9.39 mol) was added slowly. The reaction mixture was then allowed to stir at 25° C. for 5 h. After completion of the reaction, the reaction mixture was poured into ice cold water (10.0 L), and the aqueous layer was extracted with DCM (5.00 L×3). The combined organic layers were washed with a sat. aq. NaHCO₃ solution (5.00 L), brine (5.00 L), dried (Na₂SO₄), filtered and concentrated under vacuum to give the title compound as a yellow solid. ¹H NMR (600 MHz, CDCl₃) δ 10.42 (br s, 1H), 8.04 (s, 1H), 7.35 (s, 1H), 2.58 (s, 3H). UPLC-MS-1: Rt=1.02 min; MS m/z [M+H]⁺; 243/245/247.

### Step D.6: 4-bromo-5-chloro-6-methyl-1-(tetrahydro-2H-pyran-2-yl)-1H-indazole

To a solution of PTSA (89.8 g, 521 mmol) and 4-bromo-5-chloro-6-methyl-1H-indazole (Step D.5, 1.28 kg, 5.21 mol) in DCM (12.0 L) was added DHP (658 g, 7.82 mol, 715 mL) dropwise at 25° C. The mixture was stirred at 25° C. for 1 h. After completion the reaction, the reaction mixture was diluted with water (5.00 L) and the organic layer was separated. The aqueous layer was re-extracted with DCM (2.00 L). The combined organic layers were washed with a sat. aq. NaHCO₃ solution (1.50 L), brine (1.50 L), dried over Na₂SO₄, filtered and concentrated under vacuum. The crude residue was purified by normal phase chromatography (eluent: Petroleum ether/EtOAc from 100/1 to 10/1) to give the title compound as a yellow solid. ¹H NMR (600 MHz, DMSO-d₆) δ 8.04 (s, 1H), 7.81 (s, 1H), 5.88-5.79 (m, 1H), 3.92-3.83 (m, 1H), 3.80-3.68 (m, 1H), 2.53 (s, 3H), 2.40-2.32 (m, 1H), 2.06-1.99 (m, 1H), 1.99-1.93 (m, 1H), 1.77-1.69 (m, 1H), 1.60-1.56 (m, 2H). UPLC-MS-6: Rt=1.32 min; MS m/z [M+H]⁺; 329.0/331.0/333.0

### Step D.7: 5-chloro-6-methyl-1-(tetrahydro-2H-pyran-2-yl)-4-(4,4,5,5-tetramethyl-1,3,2-dioxaborolan-2-yl)-1H-indazole (Intermediate D.1

A suspension of 4-bromo-5-chloro-6-methyl-1-(tetrahydro-2H-pyran-2-yl)-1H-indazole (Step D.6, 450 g, 1.37 mol), KOAc (401 g, 4.10 mol) and B₂Pin₂ (520 g, 2.05 mol) in 1,4-dioxane (3.60 L) was degassed with nitrogen for 0.5 h. Pd(dppf)Cl₂·CH₂Cl₂ (55.7 g, 68.3 mmol) was added and the reaction mixture was stirred at 90° C. for 6 h. The reaction mixture was filtered through diatomite and the filter cake was washed with EtOAc (1.50 L×3). The mixture was concentrated under vacuum to give a black oil which was purified by normal phase chromatography (eluent: Petroleum ether/EtOAc from 100/1 to 10/1) to give the desired product as brown oil. The residue was suspended in petroleum ether (250 mL) for 1 h to obtain a white precipitate. The suspension was filtered, dried under vacuum to give the title compound as a white solid. ¹H NMR (400 MHz, CDCl₃) δ 8.17 (d, 1H), 7.52 (s, 1H), 5.69-5.66 (m, 1H), 3.99-3.96 (m, 1H), 3.75-3.70 (m, 1H), 2.51 (d, 4H), 2.21-2.10 (m, 1H), 2.09-1.99 (m, 1H), 1.84-1.61 (m, 3H), 1.44 (s, 12H); UPLC-MS-6: Rt=1.29 min; MS m/z [M+H]⁺; 377.1/379.

### Synthesis of Compound A

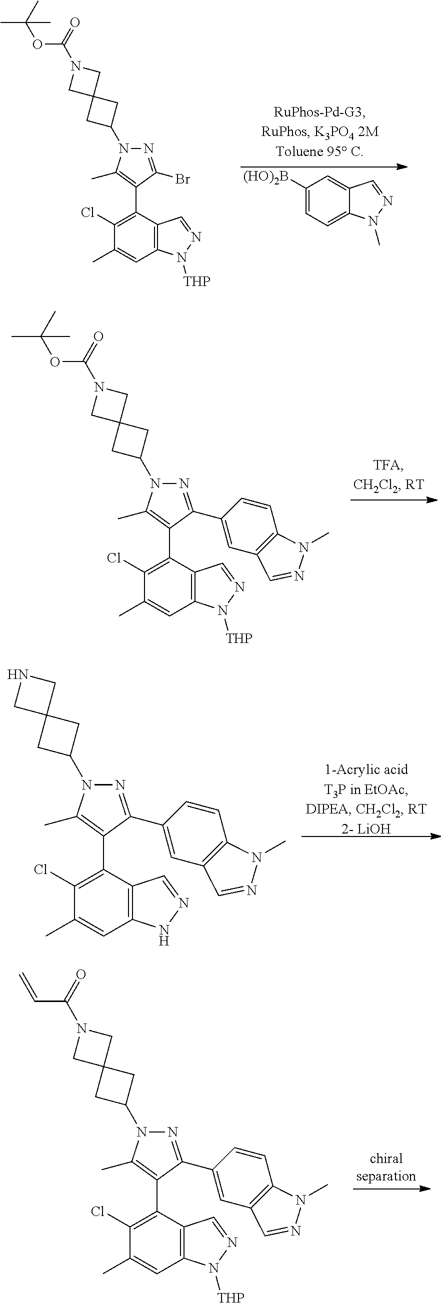

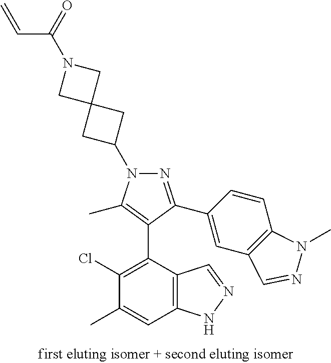 [SMILES: C=CC(=O)N1CC2(CC(n3nc(-c4ccc5c(cnn5C)c4)c(-c4c(Cl)c(C)cc5[nH]ncc45)c3C)C2)C1]

### Step 1: Tert-butyl 6-(4-(5-chloro-6-methyl-1-(tetrahydro-2H-pyran-2-yl)-1H-indazol-4-yl)-5-methyl-3-(1-methyl-TH-indazol-5-yl)-1H-pyrazol-1-yl)-2-azaspiro[3.3]heptane-2-carboxylate

In a 500 mL flask, tert-butyl 6-(3-bromo-4-(5-chloro-6-methyl-1-(tetrahydro-2H-pyran-2-yl)-1H-indazol-4-yl)-5-methyl-1H-pyrazol-1-yl)-2-azaspiro[3.3]heptane-2-carboxylate (Intermediate C1, 10 g, 16.5 mmol), (1-methyl-TH-indazol-5-yl)boronic acid (6.12 g, 33.1 mmol), RuPhos (1.16 g, 2.48 mmol) and RuPhos-Pd-G3 (1.66 g, 1.98 mmol) were suspended in toluene (165 mL) under argon. K₃PO₄ (2M, 24.8 mL, 49.6 mmol) was added and the reaction mixture was placed in a preheated oil bath (95° C.) and stirred for 45 min. The reaction mixture was poured into a sat. aq. NH₄Cl solution and was extracted with EtOAc (×3). The combined organic layers were washed with a sat. aq. NaHCO₃ solution, dried (phase separator) and concentrated under reduced pressure. The crude residue was diluted with THF (50 mL), SiliaMetS® Thiol (15.9 mmol) was added and the mixture swirled for 1 h at 40° C. The mixture was filtered, the filtrate was concentrated and the crude residue was purified by normal phase chromatography (eluent: MeOH in CH₂Cl₂ from 0 to 2%), the purified fractions were again purified by normal phase chromatography (eluent: MeOH in CH₂Cl₂ from 0 to 2%) to give the title compound as a beige foam. UPLC-MS-3: Rt=1.23 min; MS m/z [M+H]⁺; 656.3/658.3.

### Step 2: 5-Chloro-6-methyl-4-(5-methyl-3-(1-methyl-1H-indazol-5-yl)-1-(2-azaspiro[3.3]heptan-6-yl)-1H-pyrazol-4-yl)-1H-indazole

TFA (19.4 mL, 251 mmol) was added to a solution of tert-butyl 6-(4-(5-chloro-6-methyl-1-(tetrahydro-2H-pyran-2-yl)-1H-indazol-4-yl)-5-methyl-3-(1-methyl-1H-indazol-5-yl)-1H-pyrazol-1-yl)-2-azaspiro[3.3]heptane-2-carboxylate (Step 1, 7.17 g, 10.0 mmol) in CH₂Cl₂ (33 mL). The reaction mixture was stirred at RT under nitrogen for 1.5 h. The RM was concentrated under reduced pressure to give the title compound as a trifluoroacetate salt, which was used without purification in the next step. UPLC-MS-3: Rt=0.74 min; MS m/z [M+H]⁺; 472.3/474.3.

### Step 3: 1-(6-(4-(5-Chloro-6-methyl-1H-indazol-4-yl)-5-methyl-3-(1-methyl-TH-indazol-5-yl)-1H-pyrazol-1-yl)-2-azaspiro[3.3]heptan-2-yl)prop-2-en-1-one

A mixture of acrylic acid (0.69 mL, 10.1 mmol), propylphosphonic anhydride (50% in EtOAc, 5.94 mL, 7.53 mmol) and DIPEA (21.6 mL, 126 mmol) in CH₂Cl₂ (80 mL) was stirred for 20 min at RT and then added (dropping funnel) to an ice-cooled solution of 5-chloro-6-methyl-4-(5-methyl-3-(1-methyl-1H-indazol-5-yl)-1-(2-azaspiro[3.3]heptan-6-yl)-1H-pyrazol-4-yl)-1H-indazole trifluoroacetate (Step 2, 6.30 mmol) in CH₂Cl₂ (40 mL). The reaction mixture was stirred at RT under nitrogen for 15 min. The RM was poured into a sat. aq. NaHCO₃ solution and extracted with CH₂Cl₂ (×3). The combined organic layers were dried (phase separator) and concentrated. The crude residue was diluted with THF (60 mL) and LiOH (2N, 15.7 mL, 31.5 mmol) was added. The mixture was stirred at RT for 30 min until disappearance (UPLC) of the side product resulting from the reaction of the acryloyl chloride with the free NH group of the indazole then was poured into a sat. aq. NaHCO₃ solution and extracted with CH₂Cl₂ (3×). The combined organic layers were dried (phase separator) and concentrated. The crude residue was purified by normal phase chromatography (eluent: MeOH in CH₂Cl₂ from 0 to 5%) to give the title compound. The isomers were separated by chiral SFC (C-SFC-1; mobile phase: CO₂/[IPA+0.1% Et₃N]: 69/31) to give Compound A, i.e. a(R)-1-(6-(4-(5-chloro-6-methyl-1H-indazol-4-yl)-5-methyl-3-(1-methyl-1H-indazol-5-yl)-1H-pyrazol-1-yl)-2-azaspiro[3.3]heptan-2-yl)prop-2-en-1-one, as the second eluting peak (white powder): ¹H NMR (600 MHz, DMSO-d₆) δ 13.1 (s, 1H), 7.89 (s, 1H), 7.59 (s, 1H), 7.55 (s, 1H), 7.42 (m, 2H), 7.30 (d, 1H), 6.33 (m, 1H), 6.12 (m, 1H), 5.68 (m, 1H), 4.91 (m, 1H), 4.40 (s, 1H), 4.33 (s, 1H), 4.11 (s, 1H), 4.04 (s, 1H), 3.95 (s, 3H), 2.96-2.86 (m, 2H), 2.83-2.78 (m, 2H), 2.49 (s, 3H), 2.04 (s, 3H); UPLC-MS-4: Rt=4.22 min; MS m/z [M+H]⁺ 526.3/528.3; C-SFC-3 (mobile phase: CO₂/[IPA+0.1% Et₃N]: 67/33): Rt=2.23 min. The compound of Example 1 is also referred to as “Compound A”.

The atropisomer of Compound A, a(S)-1-(6-(4-(5-chloro-6-methyl-TH-indazol-4-yl)-5-methyl-3-(1-methyl-TH-indazol-5-yl)-1H-pyrazol-1-yl)-2-azaspiro[3.3]heptan-2-yl)prop-2-en-1-one was obtained as the first eluting peak: C-SFC-3 (mobile phase: CO₂/[IPA+0.1% Et₃N]: 67/33): Rt=1.55 min.

### Example 2: Compound A (JDQ443) Shows Anti-Tumor Activity in KRAS G12C-Mutated CDX Models, Driven by Target Occupancy

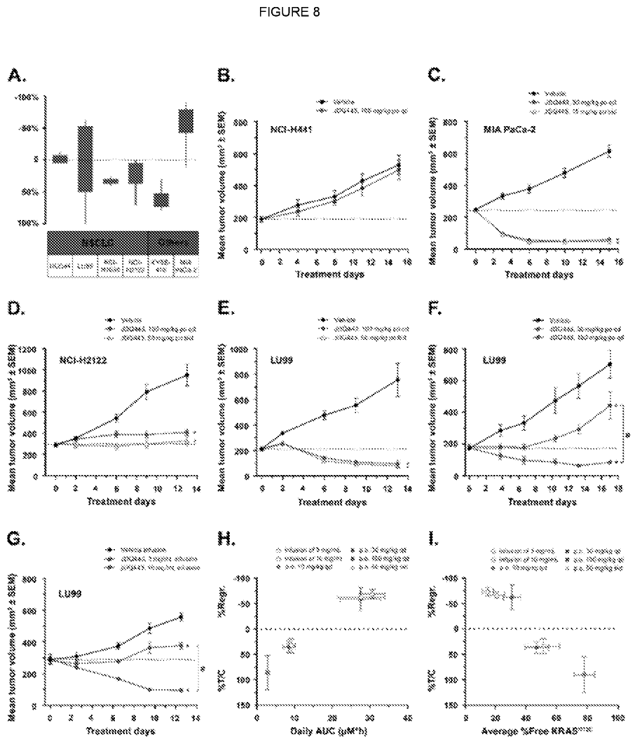

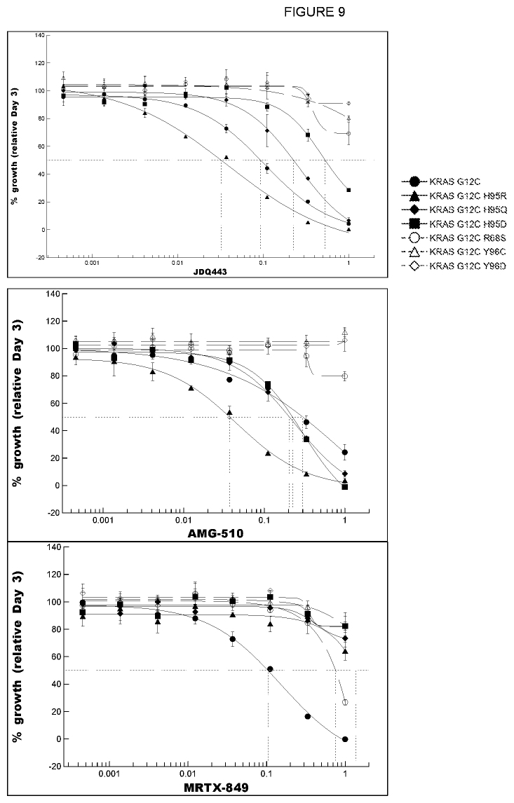

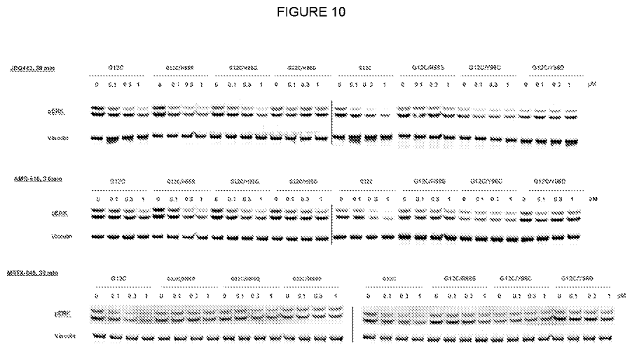

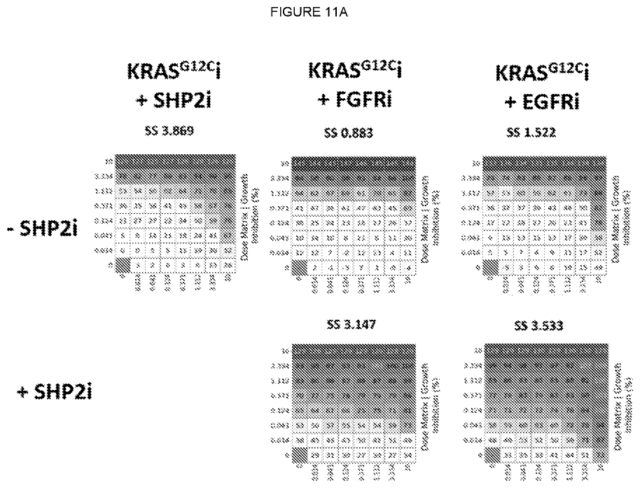

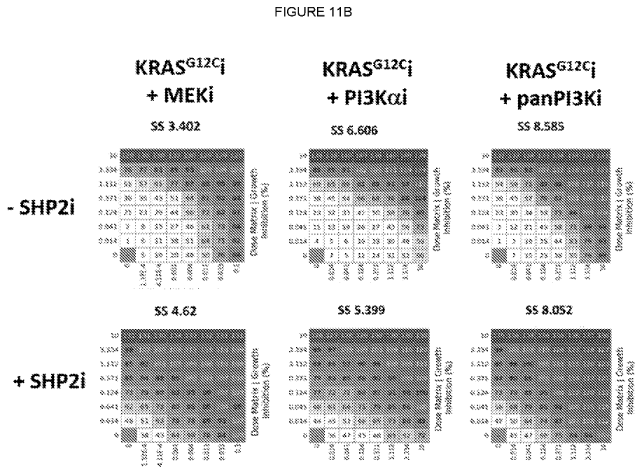

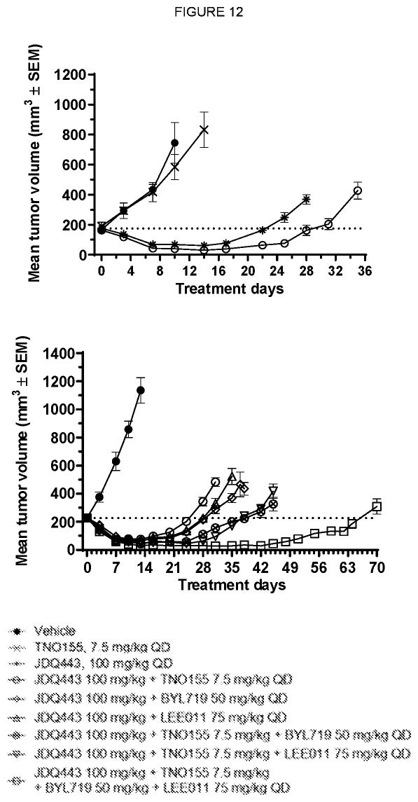

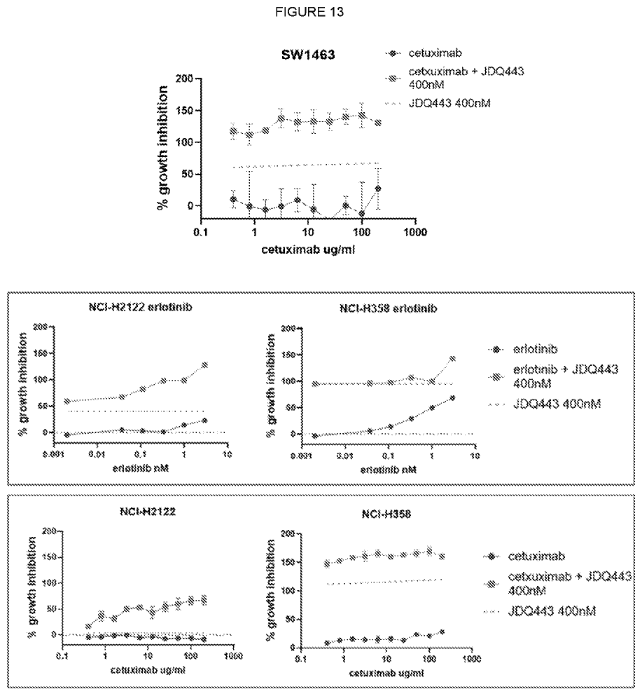

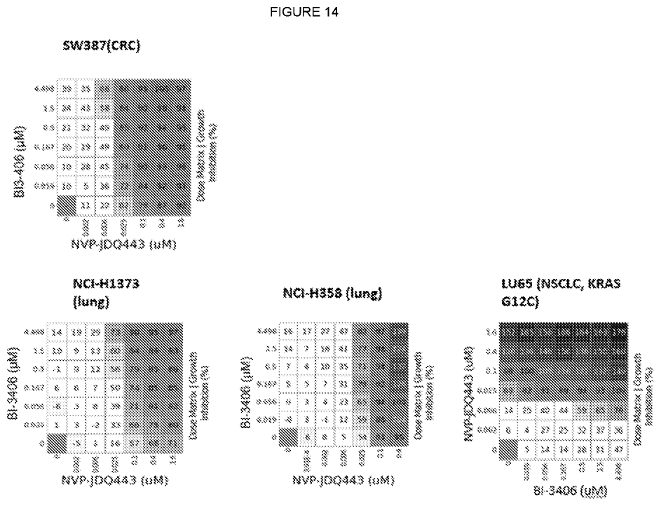

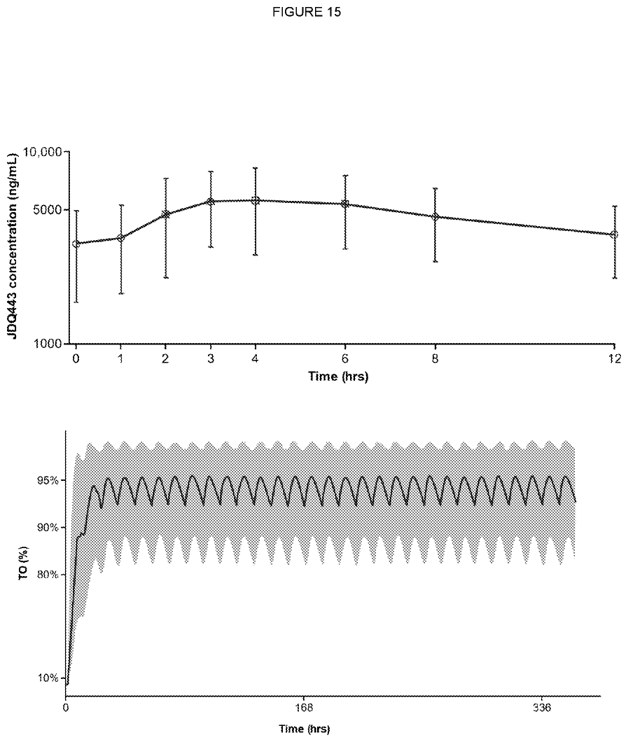

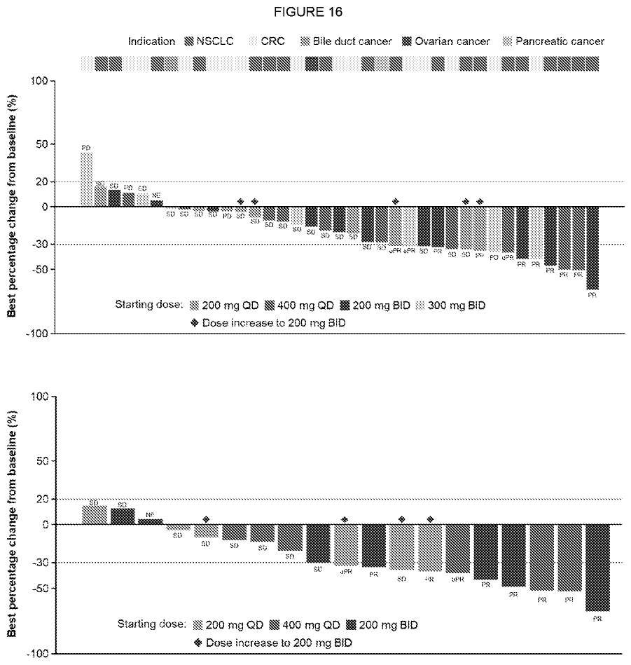

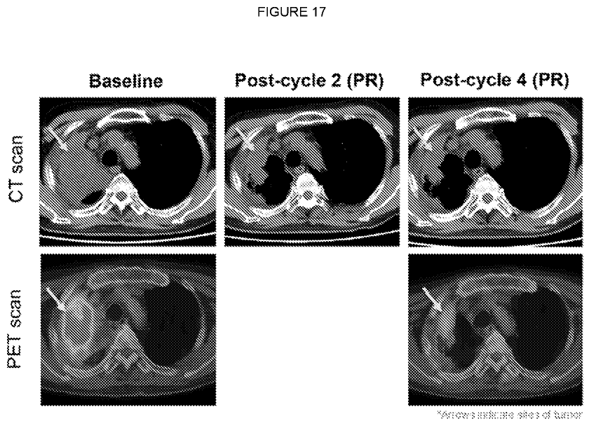

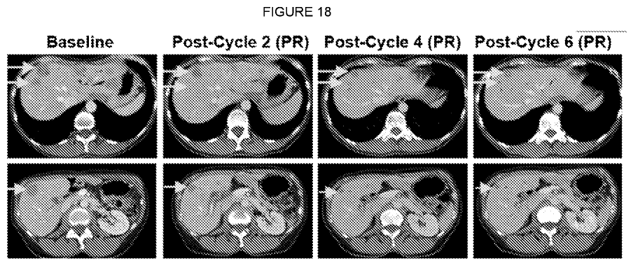

Single-agent antitumor activity of JDQ443 at daily oral doses of 10 mg/kg, 30 mg/kg and 100 mg/kg, in a panel of KRAS G12C-mutated CDX models across different indications. Cell lines for xenografting were: MIA PaCa-2 (PDAC); NCI-H2122, LU99, HCC-44, NCI-H2030 (NSCLC); and KYSE410 (esophageal cancer). JDQ443 inhibited the growth of all models in a dose-dependent manner (FIG. 8A), with model-specific differences in dose-response dynamics and maximal response patterns that ranged from regression (MIA PaCa-2, LU99), to stasis (HCC44, NCI-H2122), to moderate tumor inhibition (NCI-H2030, KYSE410). The largest dynamic range was observed in LU99. In contrast, JDQ443 showed no growth inhibition in a KRASG12V-mutated xenograft model (NCI-H441; FIG. 8B), confirming KRASG12C specificity and consistent with the in vitro data. Efficacy was maintained across once- (QD) or twice-daily (BID) administration of the same daily dose: 30 mg/kg QD versus 15 mg/kg BID in MIA PaCa-2 (FIG. 8C), or 100 mg/kg QD versus 50 mg/kg BID in NCI-H2122 and LU99 (FIG. 8D-E). The efficacy of QD vs BID dosing correlated well with comparable daily area under the concentration-time curve (AUC) in blood.

These findings suggested that JDQ443 efficacy is related to target occupancy (TO), and that efficacious AUC exposures can be achieved under both QD and BID dosing. To characterize whether AUC can act as a surrogate for TO, the effect of continuous infusion versus oral dosing in the LU99 xenograft model was investigated. Once-daily oral dosing at 30 mg/kg induced stasis for about one week followed by tumor progression, and 100 mg/kg induced tumor regression (FIG. 8F), with approximate steady-state average concentrations (Cav) of 0.3 μM and ˜1 μM, respectively. To assess continuous dosing, JDQ443 was delivered intravenously via programmable microinfusion pumps to achieve target concentrations approximating the oral Cav. Continuous infusion and oral dosing resulted in comparable antitumor responses (FIG. 8F, G). PK/PD model simulation showed that efficacy correlates best with TO and the AUC of JDQ443 (FIG. 8H, I), rather than other PK metrics.

### Example 3: Compound A Potently Inhibits KRAS G12C H95Q, a Double Mutant Mediating Resistance to Adagrasib in Clinical Trials

GFP-tagged KRASG12C H95Q, KRASG12C Y96D or KRASG12C R68S double mutations were generated by site-directed mutagenesis (QuikChange Lightning Site-Directed Mutagenesis Kit (Catalog #210518) Template: pcDNA3.1(+)EGFP-T2A-FLAG-KRAS G12C and expressed in Cas9 containing Ba/F3 cells by stable transfection. Cells were treated with a dose response curve starting at 10M with 1/3 dilution from a 10 mM DMSO stock. Cell lines were treated with indicated compounds for 72 hours and the viabilities of the cells were measured with CellTiter-Glo.

### Results:

In contrast to MRTX-849 (adagrasib), JDQ443 (Compound A) and AMG-510 (sotorasib) are potently inhibiting the cellular viability of the KRASG12C H95Q double mutant. KRASG12C Y96D or KRASG12C R68S double mutant are not inhibited by MRTX-849, AMG-510 or JDQ443 at the indicated concentrations and in the described setting (Ba/F3 system, 3-day proliferation assay) and confer resistance to all three tested KRASG12C inhibitors.

| | GI₅₀ [μM] +/− standard deviation (STDEV) | | |
|---|---|---|---|
| | KRAS G12C | KRAS G12C | KRAS G12C |
| KRAS G12C inhibitor | H95Q | Y96D | R68S |
| NVP-JDQ443 | 0.57 +/− 0.18 | >10 | >10 |
| (Compound A) | | | |
| AMG-510 (sotorasib) | 0.26 +/− 0.06 | >10 | >10 |
| MRTX-849 (adagrasib) | >10 | >10 | >10 |

### Conclusion:

Compound A might overcome resistance towards adagrasib in the KRASG12C H95Q setting. In addition, since Compound A has unique binding interactions with mutated KRAS G12C, when compared with sotorasib and adagrasib, Compound A, alone or in combination with one or more therapeutic agent as described herein, may be useful to treat patients suffering from cancer who have previously been treated with other KRAS G12C inhibitors such as sotorasib or adagrasib, or to target resistance after an acquired KRAS resistance mutation emerges on the initial KRAS G12C inhibitor treatment.

### Example 4: Compound A Potently Inhibits KRAS G12C Double Mutants

The effect of Compound A and other KRASG12C inhibitors on second-site mutations reported to confer resistance to adagrasib was also investigated as follows.

### Materials and Methods:

### Cell Lines and KRAS^{G12C} Inhibitors:

The Ba/F3 cell line is a murine pro-B-cell line and is cultured in RPMI 1640 (Bioconcept, #1-41F01-I) supplemented with 10% Fetal Bovine Serum (FBS) (BioConcept, #2-01F30-I), 2 mM Sodium pyruvate (BioConcept, #5-60F00-H), 2 mM stable Glutamine (BioConcept, #5-10K50-H), 10 mM HEPES (BioConcept, #5-31F00-H) and at 37° C. with 5% CO₂, except as otherwise indicated. The parental Ba/F3 cells were cultured in the presence of 5 ng/ml of recombinant murine IL-3 (Life Technologies, #PMC0035). Ba/F3 cells are normally dependent on IL-3 to survive and proliferate, however, by expressing oncogenes they are able to switch their dependency from IL-3 to the expressed oncogene (Curr Opin Oncology, 2007 January;19(1):55-60. doi: 10.1097/CCO.0b013e328011a25f)

### Individual Plasmid Mutagenesis and Generation of Ba/F3 Stable Cell Lines:

QuikChange Lightning Site-Directed Mutagenesis Kit (Agilent; #210519) was used to generate the resistant mutations on the pSG5_Flag-(codon optimized) KRAS^{G12C}_puro plasmid template and sequences were confirmed by sanger sequencing.

| | | SEQ ID |
|---|---|---|
| Primer | Primer sequence | NO |
| H95R_forward | 5′-gtcatttgaagatatccaccgttatcgcgagcagattaaga-3′ | 1 |
| H95R_reverse | 5′-tcttaatctgctcgcgataacggtggatatcttcaaatgac-3′ | 2 |
| H95Q_forward | 5′-tcatttgaagatatccaccagtatcgcgagcagattaagag-3′ | 3 |
| H95Q_reverse | 5′-ctcttaatctgctcgcgatactggtggatatcttcaaatga-3′ | 4 |
| H95D_forward | 5′-agtcatttgaagatatccacgattatcgcgagcagattaag-3′ | 5 |
| H95D_reverse | 5′-cttaatctgctcgcgataatcgtggatatcttcaaatgact-3′ | 6 |
| MR68S_forward | 5′-gaagagtactccgcaatgagcgatcaatacatgaggacg-3′ | 7 |
| R68S_reverse | 5′-cgtcctcatgtattgatcgctcattgcggagtactcttc-3′ | 8 |
| Y96C_forward | 5′-cgaagtcatttgaagatatccaccattgtcgcgagcagatta-3′ | 9 |
| Y96C_reverse | 5′-taatctgctcgcgacaatggtggatatcttcaaatgacttcg-3′ | 10 |
| Y96D_forward | 5′-cgaagtcatttgaagatatccaccatgatcgcgagcagatt-3 | 11 |
| Y96D_reverse | 5′-aatctgctcgcgatcatggtggatatcttcaaatgacttcg-3′ | 12 |

The mutant plasmids were transfected into the Ba/F3 WT cells by electroporation with the NEON transfection kit (Invitrogen, #MPK10025). Therefore, two million Ba/F3 cells have been electroporated with 10 μg of plasmids with the NEON System (Invitrogen, #MPK5000), using following conditions Voltage (V) 1635, Width (ms) 20, Pulses 1. After 72 h of electroporation, puromycin selection was performed at 1 μg/ml to generate stable cell lines.

### IL-3 Withdrawal

Ba/F3 cells are normally dependent on IL-3 to survive and proliferate, however, by expressing oncogenes they are able to switch their dependency from IL-3 to the expressed oncogene. To assess whether the KRAS^{G12C} single and double mutants are able to sustain the proliferation of Ba/F3 cells, the engineered Ba/F3 cells expressing the mutant constructs were cultured in absence of IL-3. Cell number and viability was measured every three days and after seven days the IL-3 withdrawal was completed. The expression of the mutants after the IL-3 withdrawal were confirmed by Western Blot (data not shown, an upwards shift was observed for KRAS^{G12C/R68S}).

Drug response curves for KRASG12C inhibitors and validation of resistance mutations: 1000 Ba/F3 cells/well were seeded at in 96-well plates (Greiner Bio-One, #655098). Treatment was performed on the same day with a Tecan D300e drug dispenser. Viability was detected on the same day of treatment for the start plate (Day 0) and three days post-treatment (Day 3) using the CellTiter-Glo Luminescent Cell Viability Assay (Promega, #G7573) on a Tecan infinitiy M200 Pro reader (Intergration Time 1000 ms).

To determine the growth, the three days post-treatment (Day 3) readout was normalized to start plate (Day 0). The percentage viability was then calculated by normalizing treated wells to DMSO treated control samples. XLfit was used to make the fitted curve with a Sigmoidal Dose-Response Model (four-parameter curve) (FIG. 9). The horizontal red dotted line represents the GI50 value. Tabular data are shown below.

TABLE
Effect of Compound A (JDQ443) on the the proliferation of KRAS G12C/H95 double
mutants. («STDEV» indicates the standard deviation for the % growth value)

% growth of
treated cells
relative to

| day 3 | conc [μM] | | | | | | | |
|---|---|---|---|---|---|---|---|---|
| treatment | 1 | 0.333333 | 0.111111 | 0.037037 | 0.012346 | 0.004115 | 0.001372 | 0.000457 |
| G12C | 4.279 | 20.252 | 44.204 | 72.785 | 89.361 | 93.832 | 97.516 | 95.501 |
| G12C | 0.961 | 0.345 | 3.567 | 3.058 | 1.072 | 0.770 | 3.921 | 3.639 |
| STDEV | | | | | | | | |
| H95R | 0.321 | 5.323 | 23.425 | 52.178 | 66.971 | 83.996 | 92.517 | 103.118 |
| H95R | 0.943 | 0.276 | 0.779 | 1.034 | 0.897 | 3.344 | 3.811 | 7.044 |
| STDEV | | | | | | | | |
| H95Q | 6.635 | 36.908 | 71.333 | 93.319 | 95.722 | 103.375 | 93.606 | 100.054 |
| H95Q | 2.025 | 0.910 | 11.656 | 4.209 | 0.919 | 6.685 | 3.996 | 2.621 |
| STDEV | | | | | | | | |
| H95D | 28.569 | 68.199 | 88.358 | 102.308 | 97.783 | 90.379 | 91.567 | 96.429 |
| H95D | 1.055 | 4.247 | 2.997 | 6.409 | 3.644 | 0.087 | 2.074 | 7.212 |
| STDEV | | | | | | | | |
| R68S | 69.159 | 93.111 | 103.721 | 108.276 | 104.608 | 100.329 | 103.390 | 100.931 |
| R68S | 7.892 | 4.607 | 9.627 | 6.839 | 0.781 | 4.288 | 2.838 | 0.996 |
| STDEV | | | | | | | | |
| Y96C | 80.229 | 91.765 | 102.592 | 104.222 | 96.167 | 105.515 | 102.424 | 109.116 |
| Y96C | 1.667 | 0.222 | 2.981 | 1.230 | 0.896 | 4.837 | 5.863 | 4.380 |
| STDEV | | | | | | | | |
| Y96D | 90.864 | 97.260 | 105.866 | 103.946 | 106.613 | 98.191 | 102.224 | 100.903 |
| Y96D | 0.852 | 0.105 | 4.943 | 6.090 | 3.050 | 1.435 | 5.305 | 3.229 |
| STDEV | | | | | | | | |

TABLE
Effect of sotorasib (AMG510) on the the proliferation of KRAS G12C/H95 double
mutants («STDEV» indicates the standard deviation for the % growth value)

% growth of
treated cells
relative to

| day 3 | conc [μM] | | | | | | | |
|---|---|---|---|---|---|---|---|---|
| treatment | 1 | 0.333333 | 0.111111 | 0.037037 | 0.012346 | 0.004115 | 0.001372 | 0.000457 |
| G12C | 24.244 | 46.199 | 71.315 | 77.193 | 92.241 | 95.211 | 94.786 | 100.303 |
| G12C | 5.785 | 4.703 | 0.727 | 0.776 | 2.500 | 1.603 | 2.016 | 5.046 |
| STDEV | | | | | | | | |
| H95R | 3.485 | 8.354 | 23.503 | 53.471 | 71.098 | 83.029 | 90.450 | 93.527 |
| H95R | 1.825 | 1.731 | 0.971 | 4.408 | 1.615 | 6.613 | 10.693 | 5.561 |
| STDEV | | | | | | | | |
| H95Q | 8.566 | 34.323 | 68.123 | 89.685 | 93.396 | 98.559 | 103.616 | 98.858 |
| H95Q | 2.203 | 1.572 | 6.416 | 5.515 | 0.970 | 1.603 | 4.610 | 1.842 |
| STDEV | | | | | | | | |
| H95D | −1.171 | 33.638 | 74.059 | 91.299 | 91.484 | 99.189 | 93.064 | 103.216 |
| H95D | 0.374 | 1.962 | 1.683 | 0.716 | 2.837 | 6.975 | 2.489 | 5.237 |
| STDEV | | | | | | | | |
| R68S | 79.748 | 94.396 | 102.811 | 98.842 | 99.936 | 100.994 | 94.166 | 95.566 |
| R68S | 3.473 | 7.672 | 2.021 | 3.308 | 0.455 | 0.885 | 1.904 | 1.680 |
| STDEV | | | | | | | | |
| Y96C | 111.964 | 105.031 | 103.341 | 97.712 | 104.892 | 109.010 | 106.167 | 103.355 |
| Y96C | 3.326 | 0.058 | 2.472 | 2.258 | 0.105 | 2.374 | 0.266 | 3.889 |
| STDEV | | | | | | | | |
| Y96D | 106.099 | 101.660 | 101.868 | 97.311 | 102.190 | 106.308 | 101.134 | 105.511 |
| Y96D | 7.943 | 8.231 | 5.850 | 1.065 | 8.679 | 8.652 | 10.362 | 3.819 |
| STDEV | | | | | | | | |

TABLE
Effect of adagrasib (MRTX-849) on the the proliferation of KRAS G12C/H95 double
mutants («STDEV» indicates the standard deviation for the % growth value)

| % growth of | | | | | | | | |
|---|---|---|---|---|---|---|---|---|
| treated cells | | | | | | | | |
| relative to | | | | | | | | |
| day 3 | conc [μM] | | | | | | | |
| treatment | 1 | 0.333333 | 0.111111 | 0.037037 | 0.012346 | 0.004115 | 0.001372 | 0.000457 |
| G12C | −0.266 | 16.329 | 51.013 | 72.820 | 87.908 | 90.538 | 97.358 | 99.591 |
| G12C | 0.026 | 0.281 | 1.081 | 5.419 | 4.200 | 3.922 | 1.669 | 7.272 |
| STDEV | | | | | | | | |
| H95R | 64.097 | 87.126 | 83.910 | 90.693 | 96.751 | 85.570 | 95.027 | 89.422 |
| H95R | 6.732 | 10.191 | 5.867 | 1.326 | 1.261 | 8.143 | 11.049 | 7.126 |
| STDEV | | | | | | | | |
| H95Q | 73.397 | 88.044 | 95.662 | 101.071 | 92.756 | 100.136 | 91.377 | 98.312 |
| H95Q | 6.148 | 0.323 | 0.148 | 0.289 | 1.657 | 1.053 | 5.052 | 1.475 |
| STDEV | | | | | | | | |
| H95D | 82.356 | 91.214 | 103.587 | 100.461 | 103.684 | 89.514 | 98.169 | 92.404 |
| H95D | 6.938 | 2.123 | 4.434 | 4.185 | 5.798 | 3.766 | 1.029 | 2.848 |
| STDEV | | | | | | | | |
| R68S | 26.823 | 84.668 | 94.008 | 101.452 | 105.903 | 100.837 | 99.142 | 97.443 |
| R68S | 2.129 | 0.233 | 1.666 | 0.085 | 7.640 | 1.368 | 3.217 | 1.746 |
| STDEV | | | | | | | | |
| Y96C | 82.638 | 96.520 | 99.562 | 102.615 | 105.187 | 101.092 | 100.220 | 99.819 |
| Y96C | 9.540 | 4.002 | 2.065 | 3.987 | 4.264 | 2.673 | 6.930 | 10.013 |
| STDEV | | | | | | | | |
| Y96D | 81.671 | 95.056 | 108.171 | 97.824 | 105.964 | 97.437 | 102.545 | 106.432 |
| Y96D | 3.884 | 2.842 | 0.058 | 4.085 | 8.415 | 7.575 | 0.349 | 6.625 |
| STDEV | | | | | | | | |

### Western Blot

After treatment with the different compounds at the indicated concentrations and for the indicated time, the cells were collected, pelleted and snap frozen at −80° C. Sixty L of lysis buffer (50 mM Tris HCl, 120 mM NaCl, 25 mM NaF, 40 mM β-glycerol phosphate disodium salt pentahydrate, 1% NP40, 1 μM microcystin, 0.1 mM Na3VO3, 0.1 mM PMSF, 1 mM DTT and 1 mM benzamidine, supplemented with 1 protease inhibitor cocktail tablet (Roche) for 10 mL of buffer) was added to each sample. The samples were then vortexed, incubated on ice for 10 min, vortexed again and centrifuged at 14000 rpm at 4° C. for 10 min. Protein concentration was determined with the BCA Protein Assay kit (Pierce, 23225). After normalization to the same total volume with lysis buffer, NuPAGE™ LDS Sample buffer 4× (Invitrogen, NP0007) and NuPAGE™ Sample reducing agent 10× (Invitrogen, NP0009) was added. The samples were heated at 70° C. for 10 min before loading on a NuPAGE™ Novex™ 4-12% Bis-Tris Midi Protein Gel, 26-wells (Invitrogen, WG1403A). Gels were run for 45 min at 200 V (PowerPac HC, Biorad) in NuPAGE MES SDS running buffer (Invitrogen, NP0002). The proteins were transferred for 7 min at 135 mA per gel on a Trans-Blot® Turbo™ Midi Nitrocellulose Transfer Packs membrane (Biorad, 1704159) using the Trans-Blot® Turbo™ system (Biorad) before staining the membrane with Ponceau red (Sigma, P7170). The membranes were blocked with TBST with 5% of milk at RT. Anti-RAS (Abcam, 108602) and anti-phospho-ERK 1/2 p44/42 MAPK (Cell Signaling, 4370) antibodies were incubated overnight at 4° C., the anti-vinculin (Sigma, V9131) antibody was incubated for 1 h at RT. Membranes were washed 3× for 5 min with TBST and the anti-rabbit (Cell Signaling, 7074) and anti-mouse (Cell Signaling, 7076) secondary antibodies were incubated for 1 h at RT. All antibodies were diluted in TBST to 1/1000, except of anti-vinculin (1/3000). Revelation was performed with WestemBright ECL (Advansta, K-12045-D20) for Ras and vinculin and with SuperSignal West Femto maximum sensitivity substrate (Thermo Fischer, 34096), on a Fusion FX (Vilber Lourmat) using the FusionCapt Advance FX7 software. (FIG. 10).

### Results

TABLE
Compound A (JDQ443) inhibits the proliferation of KRAS G12C/H95
double mutants. Ba/F3 cells expressing the indicated
FLAG-KRAS^{G12C} single or double mutants were treated with
JDQ443 (Compound A, AMG-510 (sotorasib) and MRTX-849 (adagrasib)
(8-point dilution starting at 1 mM) for 3 days and the inhibtion
of proliferation was assessed by Cell titer glo viability assay.
The average of GI₅₀ ± standard deviation (St DV) of
4 independent experiments are shown.

| GI50 ± St DV [μM] | JDQ443 | AMG-510 | MRTX-849 |
|---|---|---|---|
| G12C | 0.115 ± 0.060 | 0.389 ± 0.235 | 0.136 ± 0.071 |
| G12C/H95R | 0.024 ± 0.006 | 0.033 ± 0.008 | >1 |
| G12C/H95Q | 0.284 ± 0.041 | 0.233 ± 0.022 | >1 |
| G12C/H95D | 0.612 ± 0.151 | 0.262 ± 0.088 | >1 |
| G12C/R68S | >1 | >1 | 0.707 ± 0.165 |
| G12C/Y96C | >1 | >1 | >1 |
| G12C/Y96D | >1 | >1 | >1 |

### Biophysical Data

### Material and Methods:

### Preparation of Reagents:

### Cloning, Expression and Purification of RAS Protein Constructs

The E. coli expression constructs used in this study were based on the pET system and generated using standard molecular cloning techniques. Following the cleavable N-terminal his affinity purification tag the cDNA encoding KRAS, NRAS, and HRAS comprised aa 1-169 and was codon-optimized and synthesized by GeneArt (Thermo Fisher Scientific). Point mutations were introduced with the QuikChange Lightning Site-Directed Mutagenesis kit (Agilent). All final expression constructs were sequence verified by Sanger sequencing.

Two liters of culture medium were inoculated with a pre-culture of E. coli BL21(DE3) freshly transformed with the expression plasmid and protein expression induced with 1 mM isopropyl-β-D-thiogalactopyranoside (Sigma) for 16 hours at 18° C. Proteins with an avi-tag were transformed into E. coli harboring a compatible plasmid expressing the biotin ligase BirA and the culture medium was supplemented with 135 μM d-biotin (Sigma).

Cell pellets were resuspended in buffer A (20 mM Tris, 500 mM NaCl, 5 mM imidazole, 2 mM TCEP, 10% glycerol, pH 8.0) supplemented with Turbonuclease (Merck) and cOmplete protease inhibitor tablets (Roche). The cells were lysed by three passages through a homogenizer (Avestin) at 800-1000 bar and the lysate clarified by centrifugation at 40000 g for 40 min. The lysate was loaded onto a HisTrap HP 5 ml column (Cytiva) mounted on an AKTA Pure 25 chromatography system (Cytiva). Contaminating proteins were washed away with buffer A and bound protein was eluted with a linear gradient to buffer B (buffer A supplemented with 200 mM imidazole). During dialysis O/N the N-terminal His affinity purification tags on the non-tagged and avi-tagged proteins were cleaved off by TEV or HRV3C protease, respectively. The protein solution was re-loaded onto a HisTrap column and the flow through containing the target protein collected.

Guanosine 5′-diphosphate sodium salt (GDP, Sigma) or GppNHp-Tetralithium salt (Jena Bioscience) was added to a 24-32× molar excess over protein. EDTA (pH adjusted to 8) was added to a final concentration of 25 mM. After 1 hour at room temperature the buffer was exchanged on a PD-10 desalting column (Cytiva) against 40 mM Tris, 200 mM (NH4)2SO4, 0.1 mM ZnCl2, pH 8.0. GDP (for KRAS G12C resistance mutants H95Q/D/R, Y96D/C and R68S) or GppNHp was added to a 24-32× molar excess over protein to the eluted protein. 40 U Shrimp Alkaline Phosphatase (New England Biolabs) was added to GppNHp containing samples only. The sample was then incubated for 1 hour at 5° C. Finally, MgCl2 was added to a concentration of about 30 mM.
The protein was then further purified over a HiLoad 16/600 Superdex 200 pg column (Cytiva) pre-equilibrated with 20 mM HEPES, 150 mM NaCl, 5 mM MgCl2, 2 mM TCEP, pH 7.5. The purity and concentration of the protein was determined by RP-HPLC, its identity was confirmed by LC-MS. Present nucleotide was determined by ion-pairing chromatography [Eberth et al, 2009].

### Determination of Covalent Rate Constants by RapidFire MS

### Assay and Curve Fitting

Serial dilutions of the test compounds (50 μM, ½ dilutions) were prepared in 384 well plates and incubated with 1 μM KRAS G12C (with/without additional mutants) in 20 mM Tris pH7.5, 150 mM NaCl, 100 μM MgCl2, 1% DMSO at room temperature. Reactions were stopped at different time points by addition of formic acid to 1%. MS measurements were carried out using a Agilent 6530 quadrupole time-of-flight (QToF) MS system coupled to an Agilent RapidFire autosampler RF360 device, resulting in % modification values for each well. In parallel, compound solubility was assessed by nephelometry and compound concentrations resulting in measurable turbidity were excluded from curve fitting.

Plotting the % modification vs. time allowed for extraction of k_{obs} values for the different compound concentrations. In a second step, the obtained k_{obs} values were plotted against the compound concentrations. Rate constants (i.e. k_{inact}/K_{I}) were derived from the initial linear part of the resulting curves.

### MS Measurements

The RapidFire autosampler RF 360 was used to perform the injections. Solvents were delivered by Agilent 1200 pumps. A C18 Solid Phase Extraction (SPE) cartridge was used for all experiments.

A volume of 30 μL was aspirated from each well of a 384-well plate. The sample load/wash time was 3000 ms at a flow rate of 1.5 mL/min (H2O, 0.1% formic acid); elution time was 3000 ms (acetonitrile, 0.1% formic acid); reequilibration time was 500 ms at a flow rate of 1.25 mL/min (H2O, 0.1% formic acid).

Mass spectrometry (MS) data were acquired on an Agilent 6530 quadrupole time-of-flight (QToF) MS system, coupled to a dual Electrospray (AJS) ion source, in positive mode. The instrument parameters were as follows: gas temperature 350° C., drying gas 10 L/min, nebulizer 45 psi, sheath gas 350° C., sheath gas flow 11 L/min, capillary 4000 V, nozzle 1000 V, fragmentor 250 V, skimmer 65 V, octapole RF 750 V. Data were acquired at the rate of 6 spectra/s. The mass calibration was performed over the 300-3200 m/z range.

All data processing was performed using a combination of Agilent MassHunter Qualitative Analysis, Agilent Rapid-Fire control software, and the Agilent DA Reprocessor Offline Utilities. A Maximum Entropy algorithm produced zero-charge spectra in separate files per injection. A batch processing generated a single file incorporating all mass spectra in a text format as x,y coordinates. This file was used to calculate the % of protein modification in each well.

### Results

Quantification of the second order rate constants for modification for the indicated constructs (all GDP-loaded) was carried out using kinetic MS experiments, measuring % modification at different time points for a range of compound concentrations. K_{inact}/K_{I} was extrapolated from the initial slope of the k_{obs} vs. compound concentration plot. Activities against KRAS G12D:GDP were set to 1 and relative activities for the resistance mutants are given. Average values of n=4 experiments for KRAS G12C, n=3 for G12C_Y96D and n=2 for other mutants are given in the Table below.

TABLE
Fold change of second order rate constant (K_{inact}/K_{I}) for resistance mutants relative to KRAS G12C

| KRAS mutant | | | | | | | |
|---|---|---|---|---|---|---|---|
| (GDP) | G12C | G12C_H95R | G12C_H95Q | G12C_H95D | G12C_R68S | G12C_Y96C | G12C_Y96D |
| Compound A | 1 | 1.04 | 0.40 | 0.20 | 0.14 | 0.03 | 0.004 |
| (JDQ443) | | | | | | | |
| Sotorasib | 1 | 2.39 | 1.67 | 1.45 | 0.31 | <0.002 | <0.001 |
| Adagrasib | 1 | <0.05 | <0.05 | <0.05 | 0.38 | <0.002 | <0.001 |

Quantification of the second order rate constants for modification for the indicated constructs (all GDP-loaded) was carried out using kinetic MS experiments, measuring % modification at different time points for a range of compound concentrations. K_{inact}/K_{I} was extrapolated from the initial slope of the K_{obs} vs. compound concentration plot. Average values of n=4 experiments for KRAS G12C, n=3 for G12C_Y96D and n=2 for other mutants are given.

TABLE
Second order rate constants (K_{inact}/KI [mM−1*s−1]) for Compound
A (JDQ443), sotorasib and adagrasib against resistance mutants

| KRAS mutant | | | | | | | |
|---|---|---|---|---|---|---|---|
| (GDP) | G12C | G12C_H95R | G12C_H95Q | G12C_H95D | G12C_R68S | G12C_Y96C | G12C_Y96D |
| Compound A | 24.3 | 25 | 9.5 | 4.85 | 3.45 | 0.65 | 0.09 |
| (JDQ443) | | | | | | | |
| Sotorasib | 9 | 21.5 | 15 | 13.05 | 2.75 | <0.02 | <0.01 |
| Adagrasib | 26 | <1 | <1 | <1 | 9.9 | <0.04 | <0.01 |

### Conclusions

First generation KRAS G12C inhibitors have shown efficacy in clinical trials. However, the emergence of mutations that disrupt inhibitor binding and reactivation in downstream pathways, limits the duration of response. Second-site mutants reported to confer resistance to adagrasib in clinical trials (ref: N Engl J Med. 2021 Jun. 24; 384(25):2382-2393. doi: 10.1056/NEJMoa2105281, Cancer Discov. 2021 August; 11(8):1913-1922. doi: 10.1158/2159-8290.CD-21-0365. Epub 2021 Apr. 6.PMID: 33824136) were expressed in Ba/F3 cells and analyzed for their sensitivity towards Compound A (JDQ443) in comparison to KRAS G12C (GI₅₀=0.115±0.060 mM). As expected from the binding mode, Compound A inhibited proliferation and signaling of KRAS G12C H95 double mutants. Compound A potently inhibited the proliferation of G12C/H95R and G12C/H95Q (GI₅₀=0.024±0.006 mM, GI₅₀=0.284±0.041 mM, respectively), while expression of G12C/R68S, G12C/Y96C and G12C/Y96D conferred resistance to Compound A (GI₅₀>1 mM, all).

Surprisingly, expression of G12C/H95D resulted in reduced sensitivity to Compound A (GI₅₀=0.612±0.151 mM) compared to H95R or Q although Compound A is not directly interacting with Histidine 95. Western blot analysis of pERK upon Compound A treatment as well as the analysis of the rate constants of Compound A (biophysical data, above) towards these clinically observed SWII pocket mutations in biophysical settings were in agreement with the cellular growth inhibition data (see table).

The difference between H95D compared to H95R or Q could be due the negative charge of the aspartate, which could further increase the negative electrostatic potential of the KRAS G12C surface. This might affect ligand recognition and therefore decrease the specific reactivity and cellular activity of Compound A for this mutant. Another possible explanation is that the H95D mutation could affect KRAS dynamic so that the conformation allowing Compound A binding becomes less accessible.

In conclusion, the data show Compound A should overcome adagrasib induced resistance in G12C/Q95R or G12C/H95Q settings. Compound A treatment, particularly in combinations of the invention may still be useful in the G12C/H95Q setting where it has shown activity.

### Example 5: JDQ443 Antitumor Efficacy In Vivo is Enhanced in Combination with Inhibitors of RAS-Upstream and RAS-Downstream Signaling

The antitumor efficacy of JDQ443±inhibitors of RAS-upstream or RAS-downstream signaling was evaluated in PDX panels of human KRAS G12C-mutated NSCLC and CRC.

Patient-derived xenograft (PDX) models of human NSCLC and CRC were established by direct implantation of patient NSCLC or CRC tumor tissue subcutaneously into nude mice. PDX models were maintained through in vivo serial passaging.

A cohort of mice was implanted subcutaneously with tumor fragments from each PDX model (typically passages 4-9). Ten NSCLC and nine CRC PDX models were used. Each model is named with a code, e.g. 30580-HX, 30581-HX etc, for identification and tracking purposes. Individual mice were assigned to treatment groups or control groups for dosing once their tumor volume reached 200-250 mm³ (T=0, on the x-axis of the spider plots). One animal per PDX model was assigned to each treatment arm. Once enrolled into treatment arms, tumor volumes were measured twice weekly by caliper, and tumor volume was estimated in mm using the formula: Length×Width²/2. The end of study per model was defined as minimum of 28 days treatment, or duration for untreated tumor to reach 1500 mm³, or duration for 2 doublings of untreated tumor, whichever was slower.

Mice were treated orally with KRAS G12C inhibitor (Compound A at 100 mg/kg QD) alone or in combination with the combination partner as described in the Tables below. For example, Compound A was dosed at 100 mg/kg once daily (QD) in combination with LXH254 (naporafenib) at 50 mg/kg twice daily (BID).

Dual combinations

| Combination partner in | | |
|---|---|---|
| combination with Compound A | | |
| (Compound A at 100 mg/kg QD) | Dose and dosing schedule | |
| Raf-inhibitor (LXH254 (naporafenib)) | 50 | mg/kg twice per day (BID) |
| SHP2 inhibitor (TNO155) | 10 | mg/kg once daily (QD) |
| MEK inhibitor (trametinib) | 0.3 | mg/kg once daily (QD) |
| ERK inhibitor (LTT462 (rineterkib)) | 50 | mg/kg QD |
| CDK4/6 inhibitor (LEE011) | 75 | mg/kg QD |
| PI3K inhibitor (BYL719) | 50 | mg/kg QD |
| mTOR inhibitor (RAD001) | 10 | mg/kg QD |

Triple combinations

| Combination partner in | | |
|---|---|---|
| combination with Compound A | | |
| (Compound A at 100 mg/kg QD) | Dose and dosing schedule | |
| Raf-inhibitor (LXH254 (naporafenib)) | 50 | mg/kg twice per day (BID) |
| SHP2 inhibitor (TNO155) | 10 | mg/kg once daily (QD) |
| MEK inhibitor (trametinib) | 0.3 | mg/kg once daily (QD) |
| ERK inhibitor (LTT462 (rineterkib)) | 50 | mg/kg QD |

| | |
|---|---|
| Compound A at 100 mg/kg QD + | CDK4/6 inhibitor |
| SHP2 inhibitor (TNO155) 10 mg/kg | (LEE011)-75 mg/kg QD |
| once daily (QD) | |
| Compound A at 100 mg/kg QD + | PI3K inhibitor |
| SHP2 inhibitor (TNO155) 10 mg/kg | (BYL719)-50 mg/kg QD |
| once daily (QD) | |

Compound A and TNO155 were formulated as a suspension in 0.1% Tween 80 and 0.5% Methylcellulose in water. The Raf inhibitor (LXH254 (naporafenib)) was formulated as a suspension. The MEK inhibitor (trametinib) was formulated as a suspension in 0.2% Tween 80, 0.5% hydroxypropyl methylcellulose (HPMC), pH adjusted to pH ˜8. The ERK inhibitor (LTT462 (rineterkib)) was formulated as a suspension in 0.5% hydroxypropyl cellulose (HPC)/0.5% Pluronic in pH 7.4 phosphate-buffered saline (PBS) buffer, pH 4. The CDK4/6 inhibitor (LEE011) was formulated as a suspension in 0.5% methylcellulose. The PI3K inhibitor (BYL719) was formulated as a suspension in 0.5% Tween 80 and 1% carboxymethylcellulose in water. The mTOR inhibitor (RAD001) was formulated in 5% glucose.

The control groups were not treated.

### Results:

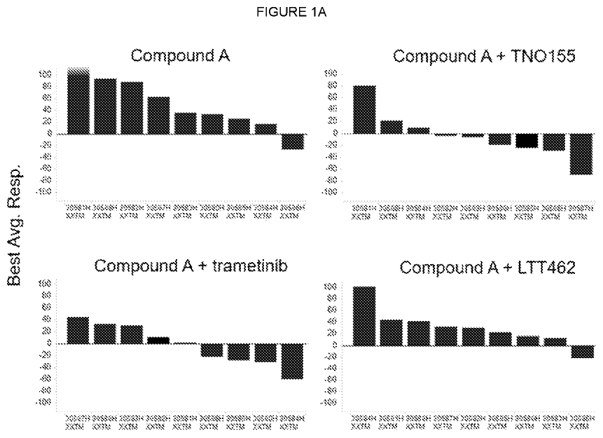

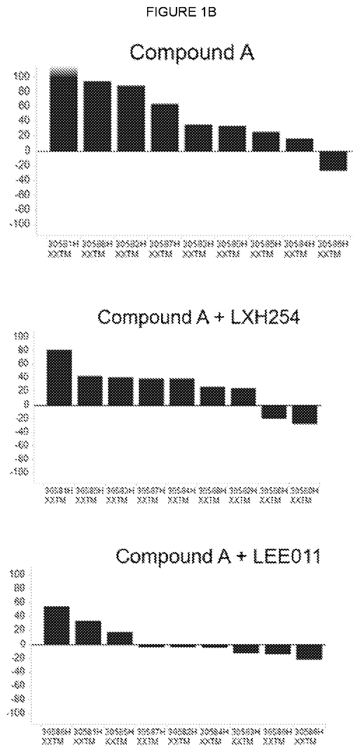

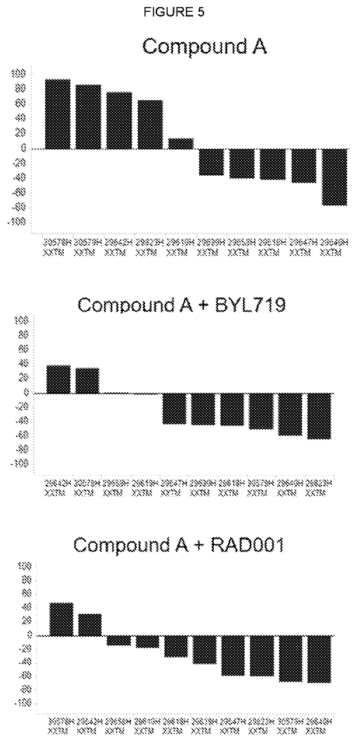

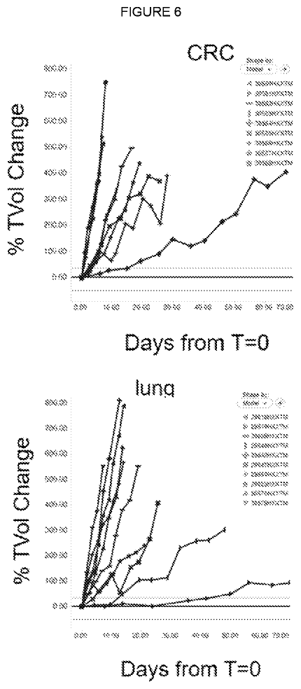

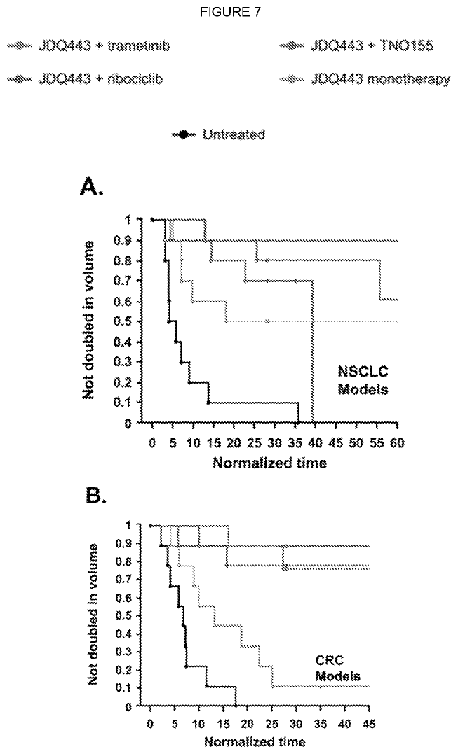

Tumor volume improvement and objective antitumor responses were greater for all combination treatments than for JDQ443 monotherapy in both the NSCLC and CRC models (FIGS. 1-6). Similarly, combination treatment benefits were observed for time to tumor volume doubling in both models (FIG. 7).

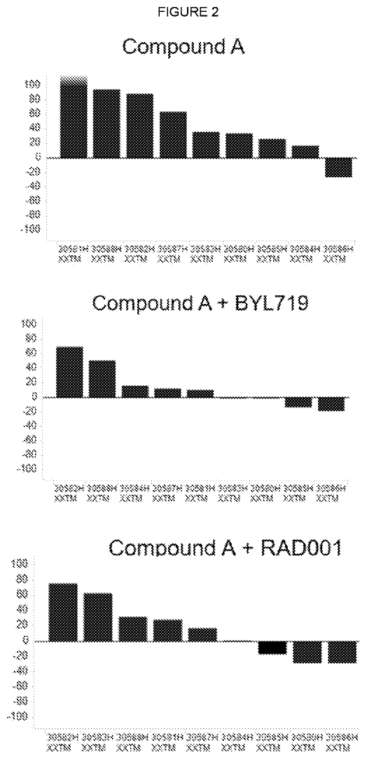

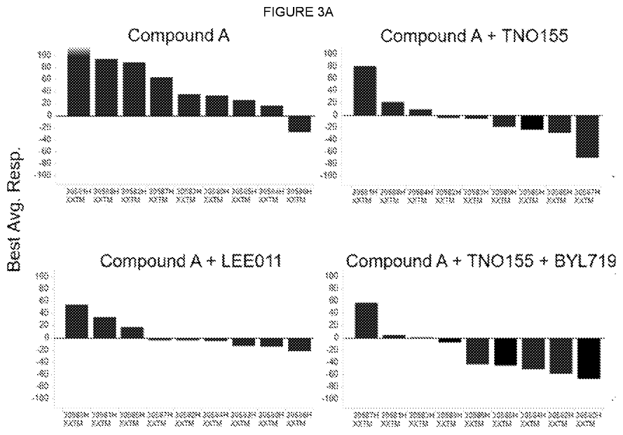

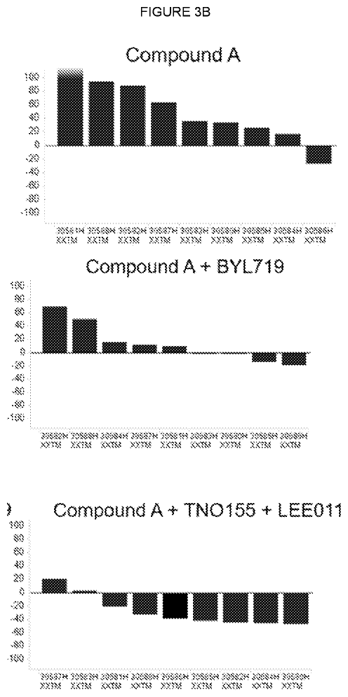

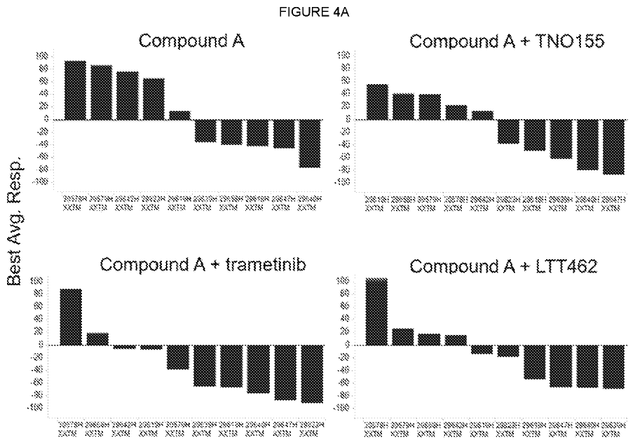

In CRC models, Compound A treatment alone caused a moderate anti-tumor response in a few models. Compound A in combination with each of the combination partners improved the anti-tumor response. Triple combinations appeared to improve the response further (FIGS. 1 and 2).

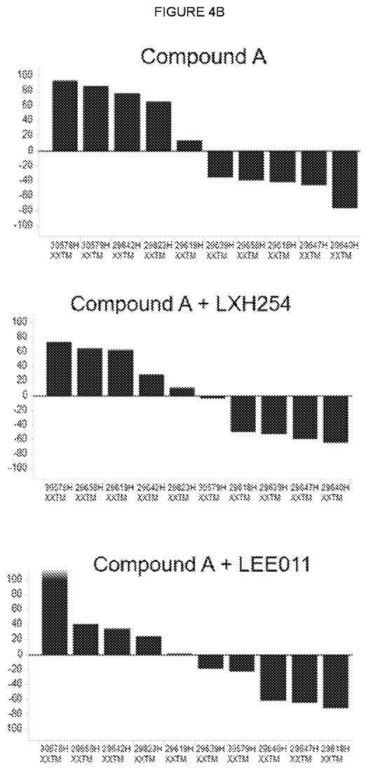

In NSCLC models, Compound A treatment alone caused no to moderate anti-tumor response in half of the models and a good anti-tumor response in the other half of the models. Compound A in combination with each of the combination partners improved the anti-tumor response (FIGS. 3, 4 and 5).

### Example 6: PI3K Inhibitors in Combination with a KRAS G12C Inhibitor Alone or in the Presence of a SHP2 Inhibitor Show Highest Synergy Scores in a 3-Day Proliferation Assay

Matrix combination proliferation assays (treatment time 3 days, cell titer glow assay) were performed with a KRAS^{G12C} inhibitor (labelled “KRAS^{G12C}i”, in FIG. 11) as single agent or in combination with 10 μM SHP099, a SHP2 inhibitor, (labelled “SHP2i” in FIG. 11) in the presence of either upstream receptor kinase inhibitors BGJ398, an FGFR inhibitor (labelled “FGFRi” in FIG. 11), and erlotinib, an EGFR inhibitor (labelled “EGFRi” in FIG. 11) or trametinib, a MEK inhibitor (labelled as “MEKi” in FIG. 11) or the PI3K effector arm inhibitors alpelisib (labelled “PI3Kαi” in FIG. 11) and GDC0941, a pan-PI3K inhibitor (labelled “panPI3Ki” in FIG. 11) in a KRAS G12C mutated H23 cell line.

Synergy scores (SS) were calculated by Loewe index and are indicated as “SS” values on top of each grid. Values in the grid are growth inhibition (%) values: a value higher than 100% indicates cell death. Growth inhibition %: 0-99=delayed proliferation, 100=growth arrest/stasis, 101-200=reduction in cell number/cell death.

The values on the x-axis of each grid indicate the concentration (in M) of the KRASG12c inhibitor used. The values on the y-axis of each grid shows the concentration (in μM) of the second agent (i.e the FGFR inhibitor, the EGFR inhibitor, the MEK inhibitor, the PI3αK inhibitor and the pan-PI3K inhibitor respectively).

As shown in FIG. 11A and FIG. 11B, the addition of a SHP2 inhibitor to a dual combination of a KRASG12C inhibitor and a second agent selected from an FGFR inhibitor, an EGFR inhibitor, a MEK inhibitor and a PI3K inhibitor increases the synergy score. For example, the synergy score increases from 1.522 for a dual combination of a KRASG12 C inhibitor and an EGFR inhibitor. to 3.533 for a triple combination of a KRASG12 C inhibitor, an EGFR inhibitor and a SHP2 inhibitor.

Highest synergy scores were obtained in the presence of a PI3K inhibitor in combination with a KRAS G12C inhibitor alone or in the presence of a SHP2 inhibitor (FIG. 11A and FIG. 11B).

### Example 7: Beneficial Eff Dose Response of JDQ443 in Combination with Erlotinib or Cetuximab in NSCLC Cell Line Sects of a Combination of Compound A and Ribociclib on a NSCLC Xenograft Model

A combination study of Compound A with ribociclib was conducted in a KRAS G12C and CDKN2A-mutated LU99 xenograft model in mice. Compound A single-agent induced tumor regression for approximately two and a half weeks, followed by tumor relapse while treatment was still ongoing. Ribociclib single-agent did not have any effect on tumor growth. The combination significantly improved the sustainability of response and time to relapse seen with Compound A as a single agent.

### Example 8: Compound A in Combination with a SHP2 Inhibitor, a PI3K Inhibitor or a CDK4/6 Inhibitor Delays Time to Progression (TPP) Compared to Single Agent Treatment with Compound A in a NSCLC Xenograft Model

An in vivo efficacy study of Compound A (JDQ443) as single agent or in combination (double, triple, quadruple) with TNO155 (a SHP2 inhibitor), BYL719 (alpelisib, a PI3K inhibitor) and LEE011 (ribociclib, a CDK4/6 inhibitor) was conducted in a KRAS G12C, PIK3CA and CDKN2A-mutated LU99 xenograft model in mice. Daily dosing with JDQ443 at 100 mg/kg induced deep tumor regression for approximately two and a half weeks, followed by tumor relapse while treatment was still ongoing. TNO155 given at 7.5 mg/kg daily did not have any effect on tumor growth compared to the vehicle group.

Double combinations of JDQ443 with TNO155, BYL719 or LEE011, triple combinations of JDQ443 and TNO155 with BYL719 or LEE011, and the quadruple combination of JDQ443 with TNO155, BYL719 and LEE011 improved the sustainability of response and time to progression seen with JDQ443 as a single agent in following order: single agent<double combination<triple combination<quadruple combination (FIG. 12).

### Example 9: Dose Response of Compound a (JDQ443) in Combination with an EGFR Inhibitor in NSCLC Cell Lines and CRC Cell Lines

A combination of cetuximab and Compound A brings additive benefit to Compound A treatment and cetuximab treatment in a CRC cell line (SW1463) (FIG. 13, top panel).

The % growth inhibition was also increased with a combination of erlotinib or cetuximab with Compound A in NSCLC (NCI-H358 and NCI-H2122) cell lines (FIG. 13 center and bottom panels).

### Example 10: Effect of Compound A. SOS-Inhibitor BI-3406 and a Combination of Compound A. SOS-Inhibitor BI-3406 on NSCLC and CRC Cell Lines

Matrix combination proliferation assays were performed as follows. For each of the cell lines, cells were dispensed into tissue culture treated 384-well plates (Greiner #781098) in a final volume of 25 L per well. Cells were allowed to adhere and begin growth for twenty-four hours. On plate was counted prior treatment (=Day 1), and the other plate was treated with compounds or DMSO using a HP D300 digital dispenser. After seventy-two hours the medium was refreshed by supplementing 25 μl per well of culture medium containing the corresponding compounds or DMSO. All treatments were done in triplicates.

Seven days after treatment initiation, cell growth was determined using CellTiter-Glo® (Promega #G7573), which measures the amount of ATP in the well. Plates were equilibrated to room temperature for approximately thirty minutes and one volume of CellTiter-Glo® Reagent equal to the volume of cell culture medium was added. Cell lysis was induced for two minutes on an orbital shaker, the plates were incubated at room temperature for ten minutes, and luminescence was recorded.

Cells were treated with the indicated final concentrations of compounds. Dose response curves were derived using XLfit dose response one site, model 205. Reported is the percentage of growth inhibition versus DMSO (percentage GI) after subtracting the reads of Day 1.

Low growth inhibition was observed with single agent treatment with SOS-inhibitor BI-3406. Combination benefit was observed with the addition of a KRAS G12C inhibitor (FIG. 14).

### Example 11: Clinical Efficacy of Compound a as Monotherapy and Combination Therapy

A phase Ib/II open-label, multi-center, dose escalation study of Compound A (JDQ443) alone and in combination with specific agents is conducted in patients with advanced solid tumors harboring the KRAS G12C mutation, including KRAS G12C-mutated NSCLC and KRAS G12C-mutated colorectal cancer (KontRASt-01 (NCT04699188)). The study is conducted to evaluate the antitumor efficacy, safety and tolerability of JDQ443 as a single agent and JDQ443 in combination with other agents. JDQ443+TNO155 and JDQ443+a PD1-inhibitor such as tislelizumab may be used to treat patients suffering KRAS G12C-mutated solid tumors.

Patients to be treated include patients with advanced, KRAS G12C-mutated solid tumors who have received standard-of-care therapy, or who are intolerant of or ineligible for approved therapies; or, Eastern Cooperative Oncology Group Performance Status (ECOG PS 0-1); or had no prior treatment with KRAS^{G12C} inhibitors. Key exclusion criteria for the JDQ443 monotherapy arm are: active brain metastases and/or prior KRASG12C inhibitor treatment.

Patients with NSCLC include patients previously treated with a platinum-based chemotherapy regimen and an immune checkpoint inhibitor, either in combination or in sequence, unless ineligible to receive such therapy.

Patients with CRC include patients who have previously received standard-of-care therapy, including fluoropyrimidine-, oxaliplatin-, and irinotecan-based chemotherapy, unless ineligible to receive such therapy.

The preliminary data from the monotherapy dose escalation arm study are as follows.

At a cut-off date of Jan. 5, 2022, 39 patients were treated with 200 mg QD, 400 mg QD, 200 mg BID or 300 mg BID of Compound A. Compound A was administered with food.

Patients had a median of 3 prior lines of anti-neoplastic therapy. The recommended dose for the monotherapy is a dose of 200 mg of Compound A taken orally twice daily (BID). Efficacy data (cutoff of 5 Jan. 2022) from the pooled Phase Ib JDQ443 single agent cohort (n=39) showed:
57% (4/7) confirmed overall response rate (ORR) at 200 mg BID in NSCLC
45% (9/20) confirmed and unconfirmed ORR across doses in NSCLC
35% (7/20) confirmed ORR across doses in NSCLC
PD/PK modeling predicted sustained, high-level target occupancy at the recommended dose of 200 mg BID

Compound A treatment was generally well tolerated. Most treatment-related adverse events (TRAEs) were Grade (Gr) 1-2. There were no Grade 4-5 TRAEs. Four Grade 3 TRAEs occurred in 4 separate pts. The most common TRAEs were fatigue, nausea, edema, diarrhea, and vomiting. There was one DLT (Grade 3 fatigue) and one treatment-related serious AE (Grade 3 photosensitivity reaction), each in separate patients treated at 300 mg BID.

| | JDQ443 200 | | | JDQ443 all | | |
|---|---|---|---|---|---|---|
| | mg BID | | | patients | | |
| | (n = 11) | | | (N = 39) | | |
| | All grades | | Grade ≥3 | All grades | | Grade ≥3 |
| Number of patients | 8 | (72.7) | 0 | 28 | (71.8) | 5 (12.8) |
| with at least one | | | | | | |
| event, n (%) | | | | | | |
| Fatigue | 2 | (18.2) | 0 | 12 | (30.8) | 1 (2.6) |
| Nausea | 3 | (27.3) | 0 | 7 | (17.9) | 0 |
| Edema | 1 | (9.1) | 0 | 6 | (15.4) | 0 |
| Diarrhea | 1 | (9.1) | 0 | 5 | (12.8) | 0 |
| Vomiting | 3 | (27.3) | 0 | 5 | (12.8) | 0 |
| Arthralgia | 1 | (9.1) | 0 | 4 | (10.3) | 0 |
| Pruritus | 1 | (9.1) | 0 | 4 | (10.3) | 0 |
| Decreased appetite | 1 | (9.1) | 0 | 3 | (7.7) | 0 |
| ALT increased | 0 | | 0 | 2 | (5.1) | 0 |
| Alopecia | 0 | | 0 | 2 | (5.1) | 0 |
| AST increased | 0 | | 0 | 2 | (5.1) | 0 |
| Dizziness | 1 | (9.1) | 0 | 2 | (5.1) | 0 |
| Dry mouth | 0 | | 0 | 2 | (5.1) | 0 |
| Dry skin | 0 | | 0 | 2 | (5.1) | 0 |
| ECG QT prolonged | 0 | | 0 | 2 | (5.1) | 0 |
| Headache | 0 | | 0 | 2 | (5.1) | 0 |
| Lipase increased | 1 | (9.1) | 0 | 2 | (5.1) | 0 |
| Musculoskeletal | 0 | | 0 | 2 | (5.1) | 0 |
| stiffness | | | | | | |
| Neuropathy | 0 | | 0 | 2 | (5.1) | 0 |
| peripheral | | | | | | |
| Neutropenia | 0 | | 0 | 2 | (5.1) | 1 (2.6) |
| Photosensitivity | 0 | | 0 | 2 | (5.1) | 2 (5.1) |
| reaction | | | | | | |
| Rash | 1 | (9.1) | 0 | 2 | (5.1) | 0 |
| (maculopapular) | | | | | | |

| | | | | | |
|---|---|---|---|---|---|
| Rash (pustular) | 0 | 0 | 2 | (5.1) | 0 |

At the recommended dose of 200 mg BID, there was prolonged absorption, with a median time to maximum plasma concentration (Tmax) of 3-4 hrs following administration with food. No significant accumulation was observed at steady state, and there was no evidence of auto-induction. The half-life was about 7 hours, and steady-state area under the curve (AUCss) was more than threefold above the exposure required for maximum efficacy in less-sensitive KRAS G12C xenograft models. FIG. 15 shows the PK profile at steady state.

| Tₘₐₓ (hrs), median | Cₘₐₓ, ss (ng/mL), | AUC₀₋₁₂, ss (ng*hr/mL), |
|---|---|---|
| (min-max) | GeoMean (CV %) | GeoMean (CV %) |
| 3.2 (2.0-7.8) | 5950 (35.0) | 49,400 (39.0) |

The predicted target occupancy profile is shown in FIG. 15. Patient PK and preclinical target occupancy models were integrated to predict target occupancy in patients at >90% in >82% patients. The models assume that JDQ443 binding and target (KRAS) turn-over rates are the same in mice and humans (˜25 hr half-life for KRAS) and that only free drug can bind the target.

The best overall response across dose levels and indications is shown in the top half of FIG. 16 and in the Table below.

| Best overall response, investigator assessed | | |
|---|---|---|
| per Response Evaluation Criteria in Solid | All patients, | |
| Tumors version 1.1 (RECIST v1.1) | N = 39, n (%) | |
| Partial response (PR) (confirmed) | 8 | (20.5) |
| Stable disease (SD) | 24 | (61.5) |
| Progressive disease (PD) | 5 | (12.8) |
| Not evaluable (NE) | 2 | (5.1) |
| Overall response rate (ORR) | 11 | (28.2) |
| (confirmed and unconfirmed) | | |
| ORR (confirmed) | 8 | (20.5) |

The best overall response across dose levels in all patients with NSCLC is shown in the bottom half of FIG. 16 and in the Table below. All patients with a Partial Response or unconfirmed Partial Response were ongoing treatment at the data cut-off.

| Best overall response, investigator | All patients with NSCLC, | |
|---|---|---|
| assessed per RECIST v1.1 | n = 20, n (%) | |
| PR (confirmed) | 7 | (35.0) |
| SD | 11 | (55.0) |
| PD | 0 | |
| NE | 2 | (10.0) |
| ORR (confirmed | 9 | (45.0) |
| and unconfirmed) | | |
| ORR (confirmed) | 7 | (35.0) |

NE, not evaluable; NSCLC, non-small cell lung cancer; ORR, overall response rate; PD, progressive disease; PR, partial response; QD, once daily.

Responses are investigator assessed per RECIST v1.1. Two (10.0%) patients had a uPR, which contributed toward the ORR (confirmed and unconfirmed).

uPR=unconfirmed PR pending confirmation, treatment ongoing with no PD. One of two patients with a uPR had confirmed PR after the data cut-off
FIG. 17 shows PET scans showing a substantial reduction in the 2-[fluorine-18]-fluoro-2-deoxy-d-glucose (18-F-FDG) avidity of the tumor mass after four cycles of treatment with Compound A administered at 200 mg BID to a patient with NSCLC. The patient had received pemetrexed/pembrolizumab, docetaxel, tegafur/gimeracil/oteracil, and carboplatin/paclitaxel/atezolizumab. Post-Cycle 2 scan showed a 30.4% reduction in the sum of the longest diameters of target lesions compared with baseline. PR was confirmed on subsequent scans
   The combination of Compound A and a SHP2 inhibitor such as TNO155 also showed clinical efficacy. FIG. 18 shows a post-cycle 2 scan from a patient with KRAS G12C-mutated duodenal papillary cancer and who had previously treated with cisplatin/gemcitabine and tegafur, each with a best response of progressive disease. The patient was treated with JDQ443 200 mg QD continuously and TNO155 20 mg QD 2 weeks on/1 week off. The post-cycle 2 scan showed a 44.2% reduction in the sum of the longest diameters of target lesions compared with baseline.
   Two cases of patients treated in the first-in-human clinical trial are provided here to illustrate the clinical antitumor activity of JDQ443 alone or with TNO155 (FIG. 17 and FIG. 18).

Case 1: a 57 year old male with metastatic KRAS G12C-mutated NSCLC. Local molecular testing using next generation sequencing (NGS) identified no mutations in TP53. Mutation status of STK11, KEAP1 and NRF2 were unknown. The patient had received prior carboplatin/pemetrexed/pembrolizumab, docetaxel, tegafur-gimeracil-oteracil, and carboplatin/paclitaxel/atezolizumab. He was enrolled to the JDQ443 monotherapy dose escalation part of the study at a dose of JDQ443 200 mg BID given continuously on a 21-day cycle. Disease assessment after 2 cycles of treatment demonstrated a RECIST 1.1 partial response, with a −30.4% change in the sum of the longest diameters of target lesions compared with baseline. Partial response was confirmed on subsequent scans (FIG. 17) and the patient continued on treatment. Positron emission tomography imaging at baseline and after 4 cycles of treatment also showed substantial reduction in 2-[fluorine-18]-fluoro-2-deoxy-d-glucose avidity of the tumor mass.

Case 2: a 58 year old female with KRAS G12C-mutated duodenal papillary cancer metastatic to liver. An R175H mutation in TP53 was observed by NGS (Foundation One panel). The patient had received prior treatment with cisplatin/gemcitabine and tegafur, both with a best response of progressive disease. She was enrolled to the dose escalation portion of the study's JDQ443+TNO155 arm, and received JDQ443 200 mg QD continuously with TNO155 20 mg QD 2 weeks on/2 weeks off. Disease assessment after two cycles of treatment demonstrated a RECIST 1.1 partial response, with a −44.2% change in the sum of the longest diameters of target lesions compared to baseline (FIG. 18). Partial response was confirmed on subsequent scans and the patient continued on treatment.

| | | | | AUC_{0-24 h} |
|---|---|---|---|---|
| | | Tₘₐₓ | Cₘₐₓ | (h × |
| Case | Treatment | (hr) | (ng/mL) | ng/mL) |
| 1 | JDQ443 200 mg BID continuously | 7.8 | 5920 | 75930 |
| 2 | JDQ443 200 mg QD continuously + | 3.2 | 5270 | 55670 |
| | TNO155 20 mg QD 2 wk on/1 wk off | | | |

### Example 12: Clinical Study Investigating Compound A Versus Docetaxel in Patients with Previously Treated, Locally Advanced or Metastatic KRAS G12C-Mutated NSCLC

An open label study which is designed to compare Compound A as monotherapy to docetaxel in participants with advanced non-small cell lung cancer (NSCLC) harboring a KRAS G12C mutation who have been previously treated with a platinum-based chemotherapy and immune checkpoint inhibitor therapy either in sequence or in combination may be carried out. The study consists of 2 parts:
Randomized part will evaluate the efficacy and safety of Compound A as monotherapy in comparison with docetaxel.
Extension part will be open after final progression-free survival (PFS) analysis (if the primary endpoint has met statistical significance) to allow participants randomized to docetaxel treatment to crossover to receive Compound A treatment.

The study population include adult participants with locally advanced or metastatic (stage IIIB/IIIC or IV) KRAS G12C mutant non-small cell lung cancer who have received prior platinum-based chemotherapy and prior immune checkpoint inhibitor therapy administered either in sequence or as combination therapy.

Participants are treated with Compound A or docetaxel following local guidelines as per standard of care and product labels (docetaxel concentrated solution for infusion, intravenously administered)

### Primary Outcome Measures Include:

### Progression Free Survival (PFS)

PFS is the time from date of randomization/start of treatment to the date of event defined as the first documented progression or death due to any cause. PFS is based on central assessment and using RECIST 1.1 criteria.

### Secondary Outcome Measures Include:

Overall Survival (OS)
OS is defined as the time from date of randomization to date of death due to any cause
Overall Response Rate (ORR)
ORR is defined as the proportion of patients with best overall response of complete response (CR) or partial response (PR) based on central and local investigator's assessment according to RECIST 1.1.
Disease Control Rate (DCR)
DCR is defined as the proportion of participants with Best Overall Response (BOR) of Complete Response (CR), Partial Response (PR), Stable Disease (SD) or Non-CR/Non-PD.
Time To Response (TTR)
TTR is defined as the time from the date of randomization to the date of first documented response (CR or PR, which must be confirmed subsequently)
Duration of Response (DOR)
DOR is calculated as the time from the date of first documented response (complete response (CR) or partial response (PR)) to the first documented date of progression or death due to underlying cancer.
Progression-Free Survival after next line therapy (PFS2)
PFS2 (based on local investigator assessment) is defined as time from date of randomization to the first documented progression on next line therapy or death from any cause, whichever occurs first.
Concentration of Compound A and its metabolite in plasma
To characterize the pharmacokinetics of Compound A and its metabolite HZC320
Time to definitive deterioration of Eastern Cooperative Group of Oncology Group (ECOG) performance status
Deterioration of Eastern Cooperative Oncology Group (ECOG) Performance Status (PS)
Time to definitive 10-point deterioration symptom scores of chest pain, cough and dyspnea per QLQ-LC13
The EORTC QLQ LC13 is a 13-item, lung cancer specific questionnaire module, and it comprises both multi-item and single-item measures of lung cancer-associated symptoms (i.e. coughing, hemoptysis, dyspnea and pain) and side-effects from conventional chemo- and radiotherapy (i.e. hair loss, neuropathy, sore mouth and dysphagia). The time to definitive 10-point deterioration is defined as the time from the date of randomization to the date of event, which is defined as at least 10 points absolute increase from baseline (worsening), with no later change below the threshold or death due to any cause
Time to definitive deterioration in global health status/QoL, shortness of breath and pain per QLQ-C30
The EORTC QLQ-C30 is a questionnaire developed to assess the health-related quality of life of cancer participants. The questionnaire contains 30 items and is composed of both multi-item scales and single-item measures based on the participants experience over the past week. These include five domains (physical, role, emotional, cognitive and social functioning), three symptom scales (fatigue, nausea/vomiting, and pain), six single items (dyspnea, insomnia, appetite loss, constipation, diarrhea and financial impact) and a global health status/HRQoL scale. The time to definitive 10-point deterioration is defined as the time from the date of randomization to the date of event, which is defined as at least 10 points absolute increase from baseline (worsening) of the corresponding scale score, with no later change below the threshold or death due to any cause
Change from baseline in EORTC-QLQ-C30
The EORTC QLQ-C30 is a questionnaire developed to assess the health-related quality of life of cancer participants. The questionnaire contains 30 items and is composed of both multi-item scales and single-item measures based on the participants experience over the past week. These include five domains (physical, role, emotional, cognitive and social functioning), three symptom scales (fatigue, nausea/vomiting, and pain), six single items (dyspnea, insomnia, appetite loss, constipation, diarrhea and financial impact) and a global health status/HRQoL scale. A higher score indicates a higher presence of symptoms.
Change from baseline in EORTC-QLQ-LC13
   The EORTC QLQ LC13 is a 13-item, lung cancer specific questionnaire module, and it comprises both multi-item and single-item measures of lung cancer-associated symptoms (i.e. coughing, hemoptysis, dyspnea and pain) and side-effects from conventional chemo- and radiotherapy (i.e. hair loss, neuropathy, sore mouth and dysphagia). A higher score indicates a higher presence of symptoms.
Change from baseline in EORTC-EQ-5D-5L
   The EQ-5D-5L is a generic instrument for describing and valuing health. It is based on a descriptive system that defines health in terms of 5 dimensions: Mobility, Self-Care, Usual Activities, Pain/Discomfort, and Anxiety/Depression.
Change from baseline in NSCLC-SAQ
   The Non-Small Cell Lung Cancer Symptom Assessment Questionnaire (NSCLC-SAQ) is a 7-item, patient-reported outcome measure which assess patient-reported symptoms associated with advanced NSCLC. It contains five domains and accompanying items that were identified as symptoms ofNSCLC: cough (1 item), pain (2 items), dyspnea (1 item), fatigue (2 items), and appetite (1 item).
PFS based on KRAS G12C mutation status in plasma
To compare the clinical outcomes for Compound A vs docetaxel based on KRAS G12C mutation status in plasma
OS based on KRAS G12C mutation status in plasma.
To compare the clinical outcomes for Compound A vs docetaxel based on KRAS G12C mutation status in plasma
ORR based on KRAS G12C mutation status in plasma.
To compare the clinical outcomes for Compound A vs docetaxel based on KRAS G12C mutation status in plasma

### Example 13: Clinical Study of JDQ443 with Select Combinations in Patients with Advanced Solid Tumors Harboring the KRAS G12C Mutation

A Phase Ib/II, multicenter, open-label platform study of JDQ443 with select combinations in patients with advanced solid tumors harboring the KRAS G12C mutation may be conducted. This study aims to characterize the safety, tolerability, pharmacokinetics, pharmacodynamics, and anti-tumor activity of JDQ443 in combination with selected therapies in adult patients with solid tumors harboring KRAS G12C mutations.

This study focuses on a single molecular subset of patients whose tumors harbor the KRAS G12C mutation and who have shown or, based on historical data, are predicted to have only modest responsiveness to single-agent KRAS G12C inhibition. The combination of JDQ443 with selected targeted therapies or other antineoplastic therapies may prevent or overcome this resistance in KRAS G12C mutant tumors, and may enable deeper and more durable responses than is historically seen with KRAS G12C inhibitor monotherapy in similar patient populations.

Each treatment arm includes a dose escalation part (Phase Ib) and a Phase II part. Dose escalations will be conducted in KRAS G12C mutant solid tumors (JDQ443+cetuximab may be be explored in CRC) to establish safety/efficacy and determine the maximum tolerated doses (MTD) and/or recommended doses (RD).

Phase II parts of the study will further explore the RD in selected indications (e.g. NSCLC and CRC for JDQ443 in combination with selected therapies). The purpose of the Phase II is to assess anti-tumor efficacy and further explore safety and tolerability of JDQ443 in combination with selected therapies at the RD(s).

| | |
|---|---|
| Key Inclusion criteria | Dose Escalation: |
| | Patients with advanced (metastatic or unresectable) KRAS G12C |
| | mutant solid tumors who have received standard of care therapy |
| | or are ineligible to receive such therapy. |
| | Phase II: |
| | Patients with advanced (metastatic or unresectable) KRAS G12C |
| | mutant non-small cell lung cancer who have received one |
| | platinum-based chemotherapy regimen and immune checkpoint |
| | inhibitor therapy, unless patient was ineligible to receive such |
| | therapy |
| | Patients with advanced (metastatic or unresectable) KRAS G12C |
| | mutant colorectal cancer who have received fluropyrimidine-, |
| | oxaliplatin-, and irinotecan-based chemotherapy, unless patient |
| | was ineligible to such therapy. |
| | All patients: |
| | ECOG performance status of 0 or 1. |
| | Patients must have a site of disease amenable to biopsy and be a |
| | candidate for tumor biopsy according to the treating institution's |
| | guidelines. |
| Key Exclusion criteria | Key exclusion applicable to all arms |
| | Tumors harboring driver mutations that have approved targeted |
| | therapies, with the exception of KRAS G12C mutations. |
| | Prior treatment with a KRAS G12C inhibitor is excluded for |
| | patients in a subset of groups in Phase II. |
| | Active brain metastases, including symptomatic brain metastases |
| | or known leptomeningeal disease |
| | Clinically significant cardiac disease or risk factors at screening |
| | Insufficient bone marrow, hepatic or renal function at screening |
| Study treatment | JDQ443, trametinib (TMT212), ribociclib (LEE011), cetuximab |
| Efficacy assessments | Tumor response assessed locally (dose escalation) and both |
| | locally and centrally (Phase II), by RECIST 1.1, every 8 weeks |
| | until week 56, then every 12 weeks. |
| | Survival status collected every 12 weeks (Phase II). |
| Pharmacokinetic | Concentration and PK parameters of JDQ443 and corresponding |
| assessments | combination partner(s), as applicable, for each treatment arm. |
| Key safety assessments | Incidence and severity of dose limiting toxicities (DLTs) of each |
| | combination treatment. |
| | Incidence and severity of adverse events (AEs) and serious |
| | adverse events (SAEs), including changes in laboratory values, |
| | electrocardiograms (ECGs), and vital signs by treatment |
| | Frequency of dose interruptions, reductions, and dose intensity, |
| | by treatment |

All publications, patents, and Accession numbers mentioned herein are hereby incorporated by reference in their entirety as if each individual publication or patent was specifically and individually indicated to be incorporated by reference.

References in this specification to “the invention” are intended to reflect embodiments of the several inventions disclosed in this specification and should not be taken as unnecessarily limiting of the claimed subject matter.

It is understood that the Examples and embodiments described herein are for illustrative purposes only and that various modifications or changes in light thereof will be suggested to persons skilled in the art and are to be included within the spirit and purview of this application and scope of the appended claims.

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1-29. (canceled)

30. A pharmaceutical combination comprising a KRAS G12C inhibitor or a pharmaceutically acceptable salt thereof, and a therapeutically active agent which is selected from the group consisting of an EGFR inhibitor, and a CDK4/6 inhibitor, and combinations thereof.

31. The pharmaceutical combination according to claim 30, wherein the additional agent is selected from
(i) an EGFR inhibitor selected from cetuximab, panitumab, afatinib, lapatinib, erlotinib, gefitinib, osimertinib and nazartinib;
(ii) a MEK inhibitor selected from pimasertib, PD-0325901, selumetinib, trametinib, binimetinib and cobimetinib; and
(iii) a CDK4/6 inhibitor selected from ribociclib, palbociclib and alemaciclib, or a pharmaceutically acceptable salt thereof.

32-39. (canceled)

40. The pharmaceutical combination according to claim 30, wherein the KRAS G12C inhibitor is selected from 1-{6-[(4M)-4-(5-chloro-6-methyl-1H-indazol-4-yl)-5-methyl-3-(1-methyl-1H-indazol-5-yl)-1H-pyrazol-1-yl]-2-azaspiro[3.3]heptan-2-yl}prop-2-en-1-one (Compound A), sotorasib, adagrasib, D-1553 and GDC6036, or a pharmaceutically acceptable salt thereof, and a therapeutically active agent which is selected from an EGFR inhibitor, a MEK inhibitor and a CDK4/6 inhibitor, and combinations thereof.

41. The pharmaceutical combination according to claim 40, wherein the KRAS G12C inhibitor is 1-{6-[(4M)-4-(5-chloro-6-methyl-1H-indazol-4-yl)-5-methyl-3-(1-methyl-1H-indazol-5-yl)-1H-pyrazol-1-yl]-2-azaspiro[3.3]heptan-2-yl}prop-2-en-1-one (Compound A).

42. The pharmaceutical combination according to claim 31, wherein the additional agent is selected from cetuximab, trametinib and ribociclib, or a pharmaceutically acceptable salt thereof.

43. The pharmaceutical combination according to claim 42, wherein the additional agent is trametinib, or a pharmaceutically acceptable salt thereof.

44. The pharmaceutical combination according to claim 42, wherein the additional agent is ribociclib, or a pharmaceutically acceptable salt thereof.

45. The pharmaceutical combination according to claim 42, wherein the additional agent is cetuximab, or a pharmaceutically acceptable salt thereof.

46. A method of treating a cancer or a tumor in a subject in need thereof, comprising administering to the subject the pharmaceutical combination of claim 29.

47. The method of claim 46, wherein the cancer or tumor is a KRAS G12C mutated cancer or tumor.

48. The method of claim 46, wherein the cancer or tumor is selected from lung cancer, colorectal cancer, pancreatic cancer, uterine cancer, rectal cancer, appendiceal cancer, small-bowel cancer, esophageal cancer, hepatobiliary cancer, bladder cancer, ovarian cancer, duodenal papillary cancer, and a solid tumor.

49. The method of claim 46, wherein the cancer or tumor is lung cancer selected from non-small cell lung cancer, colorectal cancer, pancreatic cancer, and a solid tumor.

50. The method of claim 46, wherein the therapeutic agents in the combination therapy are administered simultaneously, separately or over a period of time.

51. The method of claim 46, wherein the KRAS G12C inhibitor is 1-{6-[(4M)-4-(5-chloro-6-methyl-1H-indazol-4-yl)-5-methyl-3-(1-methyl-1H-indazol-5-yl)-1H-pyrazol-1-yl]-2-azaspiro[3.3]heptan-2-yl}prop-2-en-1-one (Compound A), or a pharmaceutically acceptable salt thereof.

52. The method of claim 51, wherein Compound A, or a pharmaceutically acceptable salt thereof, is administered at a therapeutically effective dose which is selected from 50, 100, 150, 200, 250, 300, 350, 400, 450, 500, 550 and 600 mg per day.

53. The method of claim 51, wherein Compound A, or a pharmaceutically acceptable salt thereof, is administered at a dose of 200 mg or 400 mg per day.

54. The method of claim 51, wherein Compound A, or a pharmaceutically acceptable salt thereof, is administered at a dose of 100 mg or 200 mg twice daily.

55. The method of claim 46, wherein the additional agent is selected from cetuximab, trametinib and ribociclib, or a pharmaceutically acceptable salt thereof.

56. The method according to claim 55, wherein trametinib, or a pharmaceutically acceptable salt thereof, is administered at a dose of 1 mg once a day.

57. The method according to claim 55, wherein trametinib, or a pharmaceutically acceptable salt thereof, is administered at a dose of 2 mg once a day.

58. The method according to claim 55, wherein ribociclib, or a pharmaceutically acceptable salt thereof, is administered at a dose of 200 mg once a day.

59. The method according to claim 55, wherein ribociclib, or a pharmaceutically acceptable salt thereof, is administered at a dose of 400 mg once a day.

60. The method according to claim 55, wherein ribociclib, or a pharmaceutically acceptable salt thereof, is administered once a day for a treatment period of three weeks, then one week break.

61. The method according to claim 55, wherein cetuximab, or a pharmaceutically acceptable salt thereof, is administered at a dose of 500 mg/m² once every two weeks.

62. The method according to claim 55, wherein cetuximab, or a pharmaceutically acceptable salt thereof, is administered at a dose of 400 mg/m² once every two weeks.

63. The method according to claim 55, wherein cetuximab, or a pharmaceutically acceptable salt thereof, is administered at a dose of 300 mg/m² once every two weeks.

64. The method according to claim 46, wherein the subject to be treated is selected from:
a patient suffering from a KRAS G12C mutant solid tumor, optionally wherein the patient has received and failed standard of care therapy or is intolerant or ineligible to approved therapies;
a patient suffering from KRAS G12C mutant non-small cell lung cancer (NSCLC), optionally wherein the patient who has received and failed a platinum-based chemotherapy regimen and an immune checkpoint inhibitor therapy either in combination or in sequence;
a patient suffering from KRAS G12C mutant NSCLC, optionally wherein the patient who has previously been treated with a KRAS G12C inhibitor; and
a patient suffering from KRAS G12C mutant colorectal cancer, optionally wherein the patient has received and failed standard of care therapy selected from fluoropyrimidine-based chemotherapy, oxaliplatin-based chemotherapy, and irinotecan-based chemotherapy.
